SEQUENCE LISTING (485 sequences)

SEQ ID NO: 1 - DNA, 7249 bp, Artificial sequence
  Example of a ssDNA sequence that may be used as a scaffold for origami DNA
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccc

SEQ ID NO: 2 - DNA, 7308 bp, Artificial sequence
  Example of a ssDNA sequence that may be used as a scaffold for origami DNA
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccttatacgggtactagccatgcgtatacggtcgctagcggacttgcctcgctatcaaaggtctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccc

SEQ ID NO: 3 - DNA, 7560 bp, Artificial sequence
  Example of a ssDNA sequence that may be used as a scaffold for origami DNA
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctccgtctttatcgaggtaacaagcaccacgtagcttaagccctgtttactcattacaccaaccaggaggtcagagttcggagaaatgatttatgtgaaatgcgtcagccgattcaaggcccctatattcgtgcccaccgacgagttgcttacagatggcagggccgcactgtcggtatcatagagtcactccagggcgagcgtaaatagattagaagcggggttattttggcgggacattgtcataaggttgacaattcagcactaaggacacttaagtcgtgcgcatgaattcacaaccacttagaagaacatccaccctggcttctcctgagaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccc

SEQ ID NO: 4 - DNA, 7560 bp, Artificial sequence
  Example of a ssDNA sequence that may be used as a scaffold for origami DNA
tgtgtggaattgtgagcggataacaatttcacacaggaaacagctatgaccatgattacgaattcgagctcggtacccggggatcctccgtctttatcgaggtaacaagcaccacgtagcttaagccctgtttactcattacaccaaccaggaggtcagagttcggagaaatgatttatgtgaaatgcgtcagccgattcaaggcccctatattcgtgcccaccgacgagttgcttacagatggcagggccgcactgtcggtatcatagagtcactccagggcgagcgtaaatagattagaagcggggttattttggcgggacattgtcataaggttgacaattcagcactaaggacacttaagtcgtgcgcatgaattcacaaccacttagaagaacatccaccctggcttctcctgagaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgt

SEQ ID NO: 5 - DNA, 7560 bp, Artificial sequence
  Example of a ssDNA sequence that may be used as a scaffold for origami DNA
agcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctccgtctttatcgaggtaacaagcaccacgtagcttaagccctgtttactcattacaccaaccaggaggtcagagttcggagaaatgatttatgtgaaatgcgtcagccgattcaaggcccctatattcgtgcccaccgacgagttgcttacagatggcagggccgcactgtcggtatcatagagtcactccagggcgagcgtaaatagattagaagcggggttattttggcgggacattgtcataaggttgacaattcagcactaaggacacttaagtcgtgcgcatgaattcacaaccacttagaagaacatccaccctggcttctcctgagaa

SEQ ID NO: 6 - DNA, 7560 bp, Artificial sequence
  Example of a ssDNA sequence that may be used as a scaffold for origami DNA
gggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtagcaatacttctttgattagtaataacatcacttgcctgagtagaagaactcaaactatcggccttgctggtaatatccagaacaatattaccgccagccattgcaacaggaaaaacgctcatggaaatacctacattttgacgctcaatcgtctgaaatggattatttacattggcagattcaccagtcacacgaccagtaataaaagggacattctggccaacagagatagaacccttctgacctgaaagcgtaagaatacgtggcacagacaatatttttgaatggctattagtctttaatgcgcgaactgatagccctaaaacatcgccattaaaaataccgaacgaaccaccagcagaagataaaacagaggtgaggcggtcagtattaacaccgcctgcaacagtgccacgctgagagccagcagcaaatgaaaaatctaaagcatcaccttgctgaacctcaaatatcaaaccctcaatcaatatctggtcagttggcaaatcaacagttgaaaggaattgaggaaggttatctaaaatatctttaggagcactaacaactaatagattagagccgtcaatagataatacatttgaggatttagaagtattagactttacaaacaattcgacaactcgtattaaatcctttgcccgaacgttattaattttaaaagtttgagtaacattatcattttgcggaacaaagaaaccaccagaaggagcggaattatcatcatattcctgattatcagatgatggcaattcatcaatataatcctgattgtttggattatacttctgaataatggaagggttagaacctaccatatcaaaattatttgcacgtaaaacagaaataaagaaattgcgtagattttcaggtttaacgtcagatgaatatacagtaacagtaccttttacatcgggagaaacaataacggattcgcctgattgctttgaataccaagttacaaaatcgcgcagaggcgaattattcatttcaattacctgagcaaaagaagatgatgaaacaaacatcaagaaaacaaaattaattacatttaacaatttcatttgaattaccttttttaatggaaacagtacataaatcaatatatgtgagtgaataaccttgcttctgtaaatcgtcgctattaattaattttcccttagaatccttgaaaacatagcgatagcttagattaagacgctgagaagagtcaatagtgaatttatcaaaatcataggtctgagagactacctttttaacctccggcttaggttgggttatataactatatgtaaatgctgatgcaaatccaatcgcaagacaaagaacgcgagaaaactttttcaaatatattttagttaatttcatcttctgacctaaatttaatggtttgaaataccgaccgtgtgataaataaggcgttaaataagaataaacaccggaatcataattactagaaaaagcctgtttagtatcatatgcgttatacaaattcttaccagtataaagccaacgctcaacagtagggcttaattgagaatcgccatatttaacaacgccaacatgtaatttaggcagaggcattttcgagccagtaataagagaatataaagtaccgacaaaaggtaaagtaattctgtccagacgacgacaataaacaacatgttcagctaatgcagaacgcgcctgtttatcaacaatagataagtcctgaacaagaaaaataatatcccatcctaatttacgagcatgtagaaaccaatcaataatcggctgtctttccttatcattccaagaacgggtattaaaccaagtaccgcactcatcgagaacaagcaagccgtttttattttcatcgtaggaatcattaccgcgcccaatagcaagcaaatcagatatagaaggcttatccggtattctaagaacgcgaggcgttttagcgaacctcccgacttgcgggaggttttgaagccttaaatcaagattagttgctattttgcacccagctacaattttatcctgaatcttaccaacgctaacgagcgtctttccagagcctaatttgccagttacaaaataaacagccatattatttatcccaatccaaataagaaacgattttttgtttaacgtcaaaaatgaaaatagcagcctttacagagagaataacataaaaacagggaagcgcattagacgggagaattaactgaacaccctgaacaaagtcagagggtaattgagcgctaatatcagagagataacccacaagaattgagttaagcccaataataagagcaagaaacaatgaaatagcaatagctatcttaccgaagccctttttaagaaaagtaagcagatagccgaacaaagttaccagaaggaaaccgaggaaacgcaataataacggaatacccaaaagaactggcatgattaagactccttattacgcagtatgttagcaaacgtagaaaatacatacataaaggtggcaacatataaaagaaacgcaaagacaccacggaataagtttattttgtcacaatcaatagaaaattcatatggtttaccagcgccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggttgaggcaggtcagacgattggccttgatattcacaaacaaataaatcctcattaaagccagaatggaaagcgcagtctctgaatttaccgttccagtaagcgtcatacatggcttttgatgatacaggagtgtactggtaataagttttaacggggtcagtgccttgagtaacagtgcccgtataaacagttaatgccccctgcctatttcggaacctattattctgaaacatgaaagtattaagaggctgagactcctcaagagaaggattaggattagcggggttttgctcagtaccaggcggataagtgccgtcgagagggttgatataagtatagcccggaataggtgtatcaccgtactcaggaggtttagtaccgccaccctcagaaccgccaccctcagaaccgccaccctcagagccaccaccctcattttcagggatagcaagcccaataggaacccatgtaccgtaacactgagtttcgtcaccagtacaaactacaacgcctgtagcattccacagacagccctcatagttagcgtaacgatctaaagttttgtcgtctttccagacgttagtaaatgaattttctgtatgggattttgctaaacaactttcaacagtttcagcggagtgagaatagaaaggaacaactaaaggaattgcgaataataattttttcacgttgaaaatctccaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagttaaaggccgcttttgcgggatcgtcaccctcagcagcgaaagacagcatcggaacgagggtagcaacggctacagaggctttgaggactaaagactttttcatgaggaagtttccattaaacgggtaaaatacgtaatgccactacgaaggcaccaacctaaaacgaaagaggcaaaagaatacactaaaacactcatctttgacccccagcgattataccaagcgcgaaacaaagtacaacggagatttgtatcatcgcctgataaattgtgtcgaaatccgcgacctgctccatgttacttagccggaacgaggcgcagacggtcaatcataagggaaccgaactgaccaactttgaaagaggacagatgaacggtgtacagaccaggcgcataggctggctgaccttcatcaagagtaatcttgacaagaaccggatattcattacccaaatcaacgtaacaaagctgctcattcagtgaataaggcttgccctgacgagaaacaccagaacgagtagtaaattgggcttgagatggtttaatttcaactttaatcattgtgaattaccttatgcgattttaagaactggctcattataccagtcaggacgttgggaagaaaaatctacgttaataaaacgaactaacggaacaacattattacaggtagaaagattcatcagttgagatttaggaataccacattcaactaatgcagatacataacgccaaaaggaattacgaggcatagtaagagcaacactatcataaccctcgtttaccagacgacgataaaaaccaaaatagcgagaggcttttgcaaaagaagttttgccagagggggtaatagtaaaatgtttagactggatagcgtccaatactgcggaatcgtcataaatattcattgaatccccctcaaatgctttaaacagttcagaaaacgagaatgaccataaatcaaaaatcaggtctttaccctgactattatagtcagaagcaaagcggattgcatcaaaaagattaagaggaagcccgaaagacttcaaatatcgcgttttaattcgagcttcaaagcgaaccagaccggaagcaaactccaacaggtcaggattagagagtacctttaattgctccttttgataagaggtcatttttgcggatggcttagagcttaattgctgaatataatgctgtagctcaacatgttttaaatatgcaactaaagtacggtgtctggaagtttcattccatataacagttgattcccaattctgcgaacgagtagatttagtttgaccattagatacatttcgcaaatggtcaataacctgtttagctatattttcatttggggcgcgagctgaaaaggtggcatcaattctactaatagtagtagcattaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaaagggtgagaaaggccggagacagtcaaatcaccatcaatatgatattcaaccgttctagctgataaattaatgccggagagggtagctatttttgagagatctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcattaaatttttgttaaatcagctcattttttaaccaataggaacgccatcaaaaataattcgcgtctggccttcctgtagccagctttcatcaacattaaatgtgagcgagtaacaacccgtcggattctccgtgggaacaaacggcggattgaccgtaatgggataggtcacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttctcaggagaagccagggtggatgttcttctaagtggttgtgaattcatgcgcacgacttaagtgtccttagtgctgaattgtcaaccttatgacaatgtcccgccaaaataaccccgcttctaatctatttacgctcgccctggagtgactctatgataccgacagtgcggccctgccatctgtaagcaactcgtcggtgggcacgaatataggggccttgaatcggctgacgcatttcacataaatcatttctccgaactctgacctcctggttggtgtaatgagtaaacagggcttaagctacgtggtgcttgttacctcgataaagacggaggatccccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatca

SEQ ID NO: 7 - DNA, 7704 bp, Artificial sequence
  Example of a ssDNA sequence that may be used as a scaffold for origami DNA
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcccgcctaaagagatagggccgaccaacgtcagacgcactcaatgtcacctggctgggtttgtcaggtgtcgtgactctgggcaagaccggcaactgctgttgttgagttccccatggtcggtctcagtgggaactgcttccaacttccgtctttatcgaggtaacaagcaccacgtagcttaagccctgtttactcattacaccaaccaggaggtcagagttcggagaaatgatttatgtgaaatgcgtcagccgattcaaggcccctatattcgtgcccaccgacgagttgcttacagatggcagggccgcactgtcggtatcatagagtcactccagggcgagcgtaaatagattagaagcggggttattttggcgggacattgtcataaggttgacaattcagcactaaggacacttaagtcgtgcgcatgaattcacaaccacttagaagaacatccaccctggcttctcctgagaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccc

SEQ ID NO: 8 - DNA, 7704 bp, Artificial sequence
  Example of a ssDNA sequence that may be used as a scaffold for origami DNA
agcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcccgcctaaagagatagggccgaccaacgtcagacgcactcaatgtcacctggctgggtttgtcaggtgtcgtgactctgggcaagaccggcaactgctgttgttgagttccccatggtcggtctcagtgggaactgcttccaacttccgtctttatcgaggtaacaagcaccacgtagcttaagccctgtttactcattacaccaaccaggaggtcagagttcggagaaatgatttatgtgaaatgcgtcagccgattcaaggcccctatattcgtgcccaccgacgagttgcttacagatggcagggccgcactgtcggtatcatagagtcactccagggcgagcgtaaatagattagaagcggggttattttggcgggacattgtcataaggttgacaattcagcactaaggacacttaagtcgtgcgcatgaattcacaaccacttagaagaacatccaccctggcttctcctgagaa

SEQ ID NO: 9 - DNA, 8064 bp, Artificial sequence
  Example of a ssDNA sequence that may be used as a scaffold for origami DNA
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccc

SEQ ID NO: 10 - DNA, 8064 bp, Artificial sequence
  Example of a ssDNA sequence that may be used as a scaffold for origami DNA
ggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctca

SEQ ID NO: 11 - DNA, 8100 bp, Artificial sequence
  Example of a ssDNA sequence that may be used as a scaffold for origami DNA
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccc

SEQ ID NO: 12 - DNA, 8100 bp, Artificial sequence
  Example of a ssDNA sequence that may be used as a scaffold for origami DNA
ctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagct

SEQ ID NO: 13 - DNA, 8100 bp, Artificial sequence
  Example of a ssDNA sequence that may be used as a scaffold for origami DNA
ttcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataa

SEQ ID NO: 14 - DNA, 8100 bp, Artificial sequence
  Example of a ssDNA sequence that may be used as a scaffold for origami DNA
tgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctg

SEQ ID NO: 15 - DNA, 8634 bp, Artificial sequence
  Example of a ssDNA sequence that may be used as a scaffold for origami DNA
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccattctcctgtgactcggaagtgcatttatcatctccataaaacaaaacccgccgtagcgagttcagataaaataaatccccgcgagtgcgaggattgttatgtaatattgggtttaatcatctatatgttttgtacagagagggcaagtatcgtttccaccgtactcgtgataataattttgcacggtatcagtcatttctcgcacattgcagaatggggatttgtcttcattagacttataaaccttcatggaatatttgtatgccgactctatatctataccttcatctacataaacaccttcgtgatgtctgcatggagacaagacaccggatctgcacaacattgataacgcccaatctttttgctcagactctaactcattgatactcatttataaactccttgcaatgtatgtcgtttcagctaaacggtatcagcaatgtttatgtaaagaaacagtaagataatactcaacccgatgtttgagtacggtcatcatctgacactacagactctggcatcgctgtgaagacgacgcgaaattcagcattttcacaagcgttatcttttacaaaaccgatctcactctcctttgatgcgaatgccagcgtcagacatcatatgcagatactcacctgcatcctgaacccattgacctccaaccccgtaatagcgatgcgtaatgatgtcgatagttactaacgggtcttgttcgattaactgccgcagaaactcttccaggtcaccagtgcagtgcttgataacaggagtcttcccaggatggcgaacaacaagaaactggtttccgtcttcacggacttcgttgctttccagtttagcaatacgcttactcccatccgagataacaccttcgtaatactcacgctgctcgttgagttttgattttgctgtttcaagctcaacacgcagtttccctactgttagcgcaatatcctcgttctcctggtcgcggcgtttgatgtattgctggtttctttcccgttcatccagcagttccagcacaatcgatggtgttaccaattcatggaaaaggtctgcgtcaaatccccagtcgtcatgcattgcctgctctgccgcttcacgcagtgcctgagagttaatttcgctcacttcgaacctctctgtttactgataagttccagatcctcctggcaacttgcacaagtccgacaaccctgaacgaccaggcgtcttcgttcatctatcggatcgccacactcacaacaatgagtggcagatatagcctggtggttcaggcggcgcatttttattgctgtgttgcgctgtaattcttctatttctgatgctgaatcaatgatgtctgccatctttcattaatccctgaactgttggttaatacgcatgagggtgaatgcgaataataaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccc

SEQ ID NO: 16 - DNA, 4733 bp, Artificial sequence
  Example of a ssDNA sequence that may be used as a scaffold for origami DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 17 - DNA, 35 bp, Artificial sequence
  Icosahedron monomer A core sequence
ctttattcgtaaaactagcatattcaaccgttcta

SEQ ID NO: 18 - DNA, 35 bp, Artificial sequence
  Icosahedron monomer A core sequence
agggtgagaaaggcaagaattagcaaaaatcggtt

SEQ ID NO: 19 - DNA, 42 bp, Artificial sequence
  Icosahedron monomer A core sequence
gctgataaattaatgagcataaagctaattaagcaataaagc

SEQ ID NO: 20 - DNA, 42 bp, Artificial sequence
  Icosahedron monomer A core sequence
cgatgaacggtaattcaacgcaaggataggtaaagattcaaa

SEQ ID NO: 21 - DNA, 42 bp, Artificial sequence
  Icosahedron monomer A core sequence
ctaattgggcttgactgagtaatgtgtaaaaatttttagaac

SEQ ID NO: 22 - DNA, 42 bp, Artificial sequence
  Icosahedron monomer A core sequence
gaacgagtagtacagccggagagggtaggcaaacaagagaat

SEQ ID NO: 23 - DNA, 42 bp, Artificial sequence
  Icosahedron monomer A core sequence
cctcatatatttgacctgagagtctggactatttttgagaga

SEQ ID NO: 24 - DNA, 42 bp, Artificial sequence
  Icosahedron monomer A core sequence
aataaatgcaatgcgatggtttaatttcgacgagaaacacca

SEQ ID NO: 25 - DNA, 42 bp, Artificial sequence
  Icosahedron monomer A core sequence
tctacaaaggctacataaggcttgccctaactttaatcattg

SEQ ID NO: 26 - DNA, 42 bp, Artificial sequence
  Icosahedron monomer A core sequence
gtatcaggtcattgggacagatgaacggctgaccaactttga

SEQ ID NO: 27 - DNA, 42 bp, Artificial sequence
  Icosahedron monomer A core sequence
tgaattaccttatgataagggaaccgaatgtacagaccaggc

SEQ ID NO: 28 - DNA, 42 bp, Artificial sequence
  Icosahedron monomer A core sequence
tgctcattcagtgaaacggagatttgtaaagcgcgaaacaaa

SEQ ID NO: 29 - DNA, 42 bp, Artificial sequence
  Icosahedron monomer A core sequence
gcataggctggctgccagcgattatacctcatcgcctgataa

SEQ ID NO: 30 - DNA, 42 bp, Artificial sequence
  Icosahedron monomer A core sequence
gcagacggtcaatccgattttaagaactcaacgtaacaaagc

SEQ ID NO: 31 - DNA, 35 bp, Artificial sequence
  Icosahedron monomer A core sequence
attgtgtcgaaatcttcattacccaaatggctcat

SEQ ID NO: 32 - DNA, 35 bp, Artificial sequence
  Icosahedron monomer A core sequence
ttgacccaccttcatcaagagagccggaacgaggc

SEQ ID NO: 33 - DNA, 35 bp, Artificial sequence
  Icosahedron monomer A core sequence
agatacaccttattagcgtttaaggccggaaacgt

SEQ ID NO: 34 - DNA, 35 bp, Artificial sequence
  Icosahedron monomer A core sequence
gaattagagccagcaaaaatctacgttaggtagaa

SEQ ID NO: 35 - DNA, 42 bp, Artificial sequence
  Icosahedron monomer A core sequence
caccaatgaaaccaacaacattattacaataaaacgaactaa

SEQ ID NO: 36 - DNA, 42 bp, Artificial sequence
  Icosahedron monomer A core sequence
ttcggtcatagccctaacgccaaaaggacttgagccatttgg

SEQ ID NO: 37 - DNA, 42 bp, Artificial sequence
  Icosahedron monomer A core sequence
cgtaaacagttcagatcaccgtcaccgaattacgaggcatag

SEQ ID NO: 38 - DNA, 42 bp, Artificial sequence
  Icosahedron monomer A core sequence
cctcaaatgcttgatcgatagcagcaccttttcatcggcatt

SEQ ID NO: 39 - DNA, 42 bp, Artificial sequence
  Icosahedron monomer A core sequence
taagagcaacactacagactgtagcgcggtaatcagtagcga

SEQ ID NO: 40 - DNA, 42 bp, Artificial sequence
  Icosahedron monomer A core sequence
attaaaggtgaattaaaacgagaatgacattcattgaatccc

SEQ ID NO: 41 - DNA, 42 bp, Artificial sequence
  Icosahedron monomer A core sequence
cagaatcaagttcagaatcgtcataaatcataaatcaaaaat

SEQ ID NO: 42 - DNA, 42 bp, Artificial sequence
  Icosahedron monomer A core sequence
actgcctttagcgttcataaccctcgttgacggaaattattc

SEQ ID NO: 43 - DNA, 42 bp, Artificial sequence
  Icosahedron monomer A core sequence
caggtctttaccctggaaggtaaatatttaccagacgacgat

SEQ ID NO: 44 - DNA, 42 bp, Artificial sequence
  Icosahedron monomer A core sequence
cgtccaatactgcgcggaataagtttatagaaacgcaaagac

SEQ ID NO: 45 - DNA, 42 bp, Artificial sequence
  Icosahedron monomer A core sequence
aaaaaccaaaatagtggcaacatataaatttgtcacaatcaa

SEQ ID NO: 46 - DNA, 42 bp, Artificial sequence
  Icosahedron monomer A core sequence
accgattgagggaggactattatagtcatttagactggatag

SEQ ID NO: 47 - DNA, 35 bp, Artificial sequence
  Icosahedron monomer A core sequence
tagaaaattcatatgtaatagtaaaatggaagcaa

SEQ ID NO: 48 - DNA, 35 bp, Artificial sequence
  Icosahedron monomer A core sequence
ataaaggcgagaggcttttgcaagggcgacattca

SEQ ID NO: 49 - DNA, 35 bp, Artificial sequence
  Icosahedron monomer A core sequence
ctttaatagagtcaatagtgagaaacagtacataa

SEQ ID NO: 50 - DNA, 35 bp, Artificial sequence
  Icosahedron monomer A core sequence
attaattacatttagaggaagcccgaaaaaagcga

SEQ ID NO: 51 - DNA, 42 bp, Artificial sequence
  Icosahedron monomer A core sequence
atcaatatatgtgattaattcgagcttcgacttcaaatatcg

SEQ ID NO: 52 - DNA, 42 bp, Artificial sequence
  Icosahedron monomer A core sequence
ttaagacgctgagatgctccttttgataatcaagaaaacaaa

SEQ ID NO: 53 - DNA, 42 bp, Artificial sequence
  Icosahedron monomer A core sequence
cggtttagctatatatgatgaaacaaacagaggtcatttttg

SEQ ID NO: 54 - DNA, 42 bp, Artificial sequence
  Icosahedron monomer A core sequence
ggtcaataacctttgtgaataaccttgcagcgatagcttaga

SEQ ID NO: 55 - DNA, 42 bp, Artificial sequence
  Icosahedron monomer A core sequence
cggatggcttagagatccttgaaaacatttctgtaaatcgtc

SEQ ID NO: 56 - DNA, 42 bp, Artificial sequence
  Icosahedron monomer A core sequence
ctgagcaaaagaagtttcatttggggcgtacatttcgcaaat

SEQ ID NO: 57 - DNA, 42 bp, Artificial sequence
  Icosahedron monomer A core sequence
gctattaattaaacgtttgaccattagacgagctgaaaaggt

SEQ ID NO: 58 - DNA, 42 bp, Artificial sequence
  Icosahedron monomer A core sequence
tattttcccttagacttaattgctgaatttcatttcaattac

SEQ ID NO: 59 - DNA, 42 bp, Artificial sequence
  Icosahedron monomer A core sequence
ggcatcaattctacgcagaggcgaattaataatgctgtagct

SEQ ID NO: 60 - DNA, 42 bp, Artificial sequence
  Icosahedron monomer A core sequence
aacgagtagatttagtcagatgaatatatagattttcaggtt

SEQ ID NO: 61 - DNA, 42 bp, Artificial sequence
  Icosahedron monomer A core sequence
caacatgttttaaaataaagaaattgcgcagtaacagtacct

SEQ ID NO: 62 - DNA, 42 bp, Artificial sequence
  Icosahedron monomer A core sequence
agttacaaaatcgctaatagtagtagcattcccaattctgcg

SEQ ID NO: 63 - DNA, 35 bp, Artificial sequence
  Icosahedron monomer A core sequence
tttacatcgggagacatataacagttgattaacat

SEQ ID NO: 64 - DNA, 35 bp, Artificial sequence
  Icosahedron monomer A core sequence
aacagaatatgcaactaaagttgctttgaatacca

SEQ ID NO: 65 - DNA, 35 bp, Artificial sequence
  Icosahedron monomer A core sequence
gtctcgtgggttagaacctaccattttgcggaaca

SEQ ID NO: 66 - DNA, 35 bp, Artificial sequence
  Icosahedron monomer A core sequence
ctttgcccgaacgtaaatcccgtaaaaataaagtt

SEQ ID NO: 67 - DNA, 42 bp, Artificial sequence
  Icosahedron monomer A core sequence
aagaaaccaccagattagtgatgaagggaagccgcacaggcg

SEQ ID NO: 68 - DNA, 42 bp, Artificial sequence
  Icosahedron monomer A core sequence
tctgaataatggaacgctggcagcctccactcgtattaaatc

SEQ ID NO: 69 - DNA, 42 bp, Artificial sequence
  Icosahedron monomer A core sequence
gcgccgggcgcggtcaaacaattcgacaggccagagcacatc

SEQ ID NO: 70 - DNA, 42 bp, Artificial sequence
  Icosahedron monomer A core sequence
cgcactcaatccctaggagcggaattatgtttggattatact

SEQ ID NO: 71 - DNA, 42 bp, Artificial sequence
  Icosahedron monomer A core sequence
ctcataacggaacgatataatcctgattcatcatattcctga

SEQ ID NO: 72 - DNA, 42 bp, Artificial sequence
  Icosahedron monomer A core sequence
agtattagactttatgcggtatgagccgattgcaggcgcttt

SEQ ID NO: 73 - DNA, 42 bp, Artificial sequence
  Icosahedron monomer A core sequence
ttatcagatgatatgcatcagcggggtcggtcactgttgccc

SEQ ID NO: 74 - DNA, 42 bp, Artificial sequence
  Icosahedron monomer A core sequence
atggcaattcatcatgccggacttgtagtttgaggatttaga

SEQ ID NO: 75 - DNA, 42 bp, Artificial sequence
  Icosahedron monomer A core sequence
tgcggctggtaatgcaatagataatacaaacgtcagcgtggt

SEQ ID NO: 76 - DNA, 42 bp, Artificial sequence
  Icosahedron monomer A core sequence
gctggaggtgtccactggtcagttggcacaaaccctcaatca

SEQ ID NO: 77 - DNA, 42 bp, Artificial sequence
  Icosahedron monomer A core sequence
gctggtctggtcagtgaacctcaaatataatcaacagttgaa

SEQ ID NO: 78 - DNA, 42 bp, Artificial sequence
  Icosahedron monomer A core sequence
tagattagagccgtggtaaaggtttcttgcaaccagcttacg

SEQ ID NO: 79 - DNA, 35 bp, Artificial sequence
  Icosahedron monomer A core sequence
aggaattgaggaagtggtgccatcccactgctcgt

SEQ ID NO: 80 - DNA, 35 bp, Artificial sequence
  Icosahedron monomer A core sequence
accttgccagcaaccgcaagagcactaacaactaa

SEQ ID NO: 81 - DNA, 35 bp, Artificial sequence
  Icosahedron monomer A core sequence
attatttcgctgagagccagctagtctttaatgcg

SEQ ID NO: 82 - DNA, 35 bp, Artificial sequence
  Icosahedron monomer A core sequence
aagcgtaagaatactgctggtaatatccaaaaacg

SEQ ID NO: 83 - DNA, 42 bp, Artificial sequence
  Icosahedron monomer A core sequence
cgaactgatagcccgccattgcaacaggagaacaatattacc

SEQ ID NO: 84 - DNA, 42 bp, Artificial sequence
  Icosahedron monomer A core sequence
ctgcaacagtgccaacattggcagattccccttctgacctga

SEQ ID NO: 85 - DNA, 42 bp, Artificial sequence
  Icosahedron monomer A core sequence
gcaatttaggcagacaacagagatagaaaccagtcacacgac

SEQ ID NO: 86 - DNA, 42 bp, Artificial sequence
  Icosahedron monomer A core sequence
acgccaacatgtcataaaacatcgccatagtattaacaccgc

SEQ ID NO: 87 - DNA, 42 bp, Artificial sequence
  Icosahedron monomer A core sequence
cagtaataaaagtggaggtgaggcggtctaaaaataccgaac

SEQ ID NO: 88 - DNA, 42 bp, Artificial sequence
  Icosahedron monomer A core sequence
cgggacattctggcggcattttcgagcccgccatatttaaca

SEQ ID NO: 89 - DNA, 42 bp, Artificial sequence
  Icosahedron monomer A core sequence
gaaccaccagcaatgcttaattgagaatagtaataagagaat

SEQ ID NO: 90 - DNA, 42 bp, Artificial sequence
  Icosahedron monomer A core sequence
atgaagataaaacatgataaataaggcgtttgaaataccgac

SEQ ID NO: 91 - DNA, 42 bp, Artificial sequence
  Icosahedron monomer A core sequence
ataaagtaccgacacctaaatttaatggttaaataagaataa

SEQ ID NO: 92 - DNA, 42 bp, Artificial sequence
  Icosahedron monomer A core sequence
cgctcaacagtaggaactatatgtaaatgcttaggttgggtt

SEQ ID NO: 93 - DNA, 42 bp, Artificial sequence
  Icosahedron monomer A core sequence
acaccggaatcatactttttaacctccggctgatgcaaatcc

SEQ ID NO: 94 - DNA, 42 bp, Artificial sequence
  Icosahedron monomer A core sequence
atttcatcttctgaaaaggtaaagtaatcagtataaagccaa

SEQ ID NO: 95 - DNA, 35 bp, Artificial sequence
  Icosahedron monomer A core sequence
aatcgcaagacaaaatacaaattcttactctgtcc

SEQ ID NO: 96 - DNA, 35 bp, Artificial sequence
  Icosahedron monomer A core sequence
agactacattactagaaaaagatatattttagtta

SEQ ID NO: 97 - DNA, 35 bp, Artificial sequence
  Icosahedron monomer A core sequence
aacagctaacgaaagaggcaaagacagcatcggaa

SEQ ID NO: 98 - DNA, 35 bp, Artificial sequence
  Icosahedron monomer A core sequence
gagttaaaggccgcaataattttttcacgagcctt

SEQ ID NO: 99 - DNA, 42 bp, Artificial sequence
  Icosahedron monomer A core sequence
cgagggtagcaacgaaaggctccaaaaggttgaaaatctcca

SEQ ID NO: 100 - DNA, 42 bp, Artificial sequence
  Icosahedron monomer A core sequence
aggcaccaacctaatgataccgatagttctgaggcttgcagg

SEQ ID NO: 101 - DNA, 35 bp, Artificial sequence
  Icosahedron monomer A core sequence
aaccacccctattattctgaacagtaagcgtcata

SEQ ID NO: 102 - DNA, 35 bp, Artificial sequence
  Icosahedron monomer A core sequence
cattaaagccagaatcaccggaaccagacaccctc

SEQ ID NO: 103 - DNA, 42 bp, Artificial sequence
  Icosahedron monomer A core sequence
catggcttttgatgtccctcagagccgcgccaccaccggaac

SEQ ID NO: 104 - DNA, 42 bp, Artificial sequence
  Icosahedron monomer A core sequence
tgcctatttcggaaaccagagccgccgcaacaaataaatcct

SEQ ID NO: 105 - DNA, 35 bp, Artificial sequence
  Icosahedron monomer A core sequence
caataatatcctgaatcttacgatatagaaggctt

SEQ ID NO: 106 - DNA, 35 bp, Artificial sequence
  Icosahedron monomer A core sequence
tttcatcgtaggaatcagctaatgcagagaacaag

SEQ ID NO: 107 - DNA, 42 bp, Artificial sequence
  Icosahedron monomer A core sequence
atccggtattctaaaatagataagtcctacgcgcctgtttat

SEQ ID NO: 108 - DNA, 42 bp, Artificial sequence
  Icosahedron monomer A core sequence
ccagctacaattttcggctgtctttcctcaagccgtttttat

SEQ ID NO: 109 - DNA, 35 bp, Artificial sequence
  Icosahedron monomer A core sequence
caccgccgttttttggggtcggttgagtgttgttc

SEQ ID NO: 110 - DNA, 35 bp, Artificial sequence
  Icosahedron monomer A core sequence
atcggcaaaatcccatgaatcggccaaccagggtg

SEQ ID NO: 111 - DNA, 42 bp, Artificial sequence
  Icosahedron monomer A core sequence
cagtttggaacaagtgcgtattgggcgcgcgcggggagaggc

SEQ ID NO: 112 - DNA, 42 bp, Artificial sequence
  Icosahedron monomer A core sequence
atcacccaaatcaatggccctgagagagatggtggttccgaa

SEQ ID NO: 113 - DNA, 35 bp, Artificial sequence
  Icosahedron monomer A core sequence
cgacgttccagcagttgggcgcgccatgtttacca

SEQ ID NO: 114 - DNA, 35 bp, Artificial sequence
  Icosahedron monomer A core sequence
cgggaacggataacgcgatcggtgcgggggatgtg

SEQ ID NO: 115 - DNA, 42 bp, Artificial sequence
  Icosahedron monomer A core sequence
gtcccggaatttgtagctggcgaaagggcctcttcgctatta

SEQ ID NO: 116 - DNA, 42 bp, Artificial sequence
  Icosahedron monomer A core sequence
ctgctcatttgccggtaaaacgacggccaggtggagccgcca

SEQ ID NO: 117 - DNA, 42 bp, Artificial sequence
  Icosahedron monomer A core sequence
aactttcaacagttccctcagaaccgcccctcattttcaggg

SEQ ID NO: 118 - DNA, 42 bp, Artificial sequence
  Icosahedron monomer A core sequence
cacagacagccctccagtaccaggcggagattagcggggttt

SEQ ID NO: 119 - DNA, 42 bp, Artificial sequence
  Icosahedron monomer A core sequence
atagcaagcccaataagagaaggattagtaagtgccgtcgag

SEQ ID NO: 120 - DNA, 42 bp, Artificial sequence
  Icosahedron monomer A core sequence
gtttagtaccgccatcagcggagtgagacgcctgtagcattc

SEQ ID NO: 121 - DNA, 35 bp, Artificial sequence
  Icosahedron monomer A core sequence
agggttgatataagagtacaaactacaaatagaaa

SEQ ID NO: 122 - DNA, 35 bp, Artificial sequence
  Icosahedron monomer A core sequence
actcctcaggaacccatgtacaccgtactcaggag

SEQ ID NO: 123 - DNA, 42 bp, Artificial sequence
  Icosahedron monomer A core sequence
agaactggcatgatcgcattagacgggaggtaattgagcgct

SEQ ID NO: 124 - DNA, 42 bp, Artificial sequence
  Icosahedron monomer A core sequence
cttaccgaagccctttatcccaatccaaaataaacagccata

SEQ ID NO: 125 - DNA, 42 bp, Artificial sequence
  Icosahedron monomer A core sequence
aatatcagagagattttgccagttacaaataagaaacgattt

SEQ ID NO: 126 - DNA, 42 bp, Artificial sequence
  Icosahedron monomer A core sequence
taaaaacagggaagtaagactccttattatagcaatagctat

SEQ ID NO: 127 - DNA, 35 bp, Artificial sequence
  Icosahedron monomer A core sequence
tttgtttaacgtcacaagaaacaatgaaacgcagt

SEQ ID NO: 128 - DNA, 35 bp, Artificial sequence
  Icosahedron monomer A core sequence
agcctaaaacccacaagaattcagagagaataaca

SEQ ID NO: 129 - DNA, 42 bp, Artificial sequence
  Icosahedron monomer A core sequence
acttctttgattagcaccacacccgccgggttgctttgacga

SEQ ID NO: 130 - DNA, 42 bp, Artificial sequence
  Icosahedron monomer A core sequence
acggtacgccagaaaagccggcgaacgttttagagcttgacg

SEQ ID NO: 131 - DNA, 42 bp, Artificial sequence
  Icosahedron monomer A core sequence
gcacgtataacgtgaagggagcccccgaggcgagaaaggaag

SEQ ID NO: 132 - DNA, 42 bp, Artificial sequence
  Icosahedron monomer A core sequence
acgctgcgcgtaactaataacatcacttgattttagacagga

SEQ ID NO: 133 - DNA, 35 bp, Artificial sequence
  Icosahedron monomer A core sequence
ggaagaaagcgaaaaggccgattaaagggcctgag

SEQ ID NO: 134 - DNA, 35 bp, Artificial sequence
  Icosahedron monomer A core sequence
aaccctactttcctcgttagacaagtgtagcggtc

SEQ ID NO: 135 - DNA, 42 bp, Artificial sequence
  Icosahedron monomer A core sequence
attgcgttgcgctcgggccgttttcacgctgttcttcgcgtc

SEQ ID NO: 136 - DNA, 42 bp, Artificial sequence
  Icosahedron monomer A core sequence
aaattgttatccgcagccagcggtgccgagatgccgggttac

SEQ ID NO: 137 - DNA, 42 bp, Artificial sequence
  Icosahedron monomer A core sequence
cgtgagcctcctcatcgttaacggcatcgtgccccctgcatc

SEQ ID NO: 138 - DNA, 42 bp, Artificial sequence
  Icosahedron monomer A core sequence
ggccagaatgcggcactgcccgctttccctgtttcctgtgtg

SEQ ID NO: 139 - DNA, 35 bp, Artificial sequence
  Icosahedron monomer A core sequence
agacgatccagcgcaatcatggtcatagagtcggg

SEQ ID NO: 140 - DNA, 35 bp, Artificial sequence
  Icosahedron monomer A core sequence
cagcaaacagttgaggatcccctctgtggtgctgc

SEQ ID NO: 141 - DNA, 42 bp, Artificial sequence
  Icosahedron monomer A core sequence
ggtgccggaaaccaaaaaataattcgcgttaaatgtgagcga

SEQ ID NO: 142 - DNA, 42 bp, Artificial sequence
  Icosahedron monomer A core sequence
cgcatcgtaaccgtaaatatttaaattgcaggaagattgtat

SEQ ID NO: 143 - DNA, 42 bp, Artificial sequence
  Icosahedron monomer A core sequence
gtaacaacccgtcgaaaagccccaaaaataaacgttaatatt

SEQ ID NO: 144 - DNA, 42 bp, Artificial sequence
  Icosahedron monomer A core sequence
ataggaacgccatcggcaaagcgccattttggtgtagatggg

SEQ ID NO: 145 - DNA, 35 bp, Artificial sequence
  Icosahedron monomer A core sequence
ttgttaaaattcgctgggataggtcacgcgccatt

SEQ ID NO: 146 - DNA, 35 bp, Artificial sequence
  Icosahedron monomer A core sequence
taatcaggattctccgtgggatcattttttaacca

SEQ ID NO: 147 - DNA, 46 bp, Artificial sequence
  Icosahedron monomer A non-crossover vertex staple sequence
agattcatcagttgagttttttttttttaccattagcgccatcttt

SEQ ID NO: 148 - DNA, 52 bp, Artificial sequence
  Icosahedron monomer A non-crossover vertex staple sequence
tataccagtcagtttttttttggatacgcgatttttttttaaacactcatct

SEQ ID NO: 149 - DNA, 46 bp, Artificial sequence
  Icosahedron monomer A non-crossover vertex staple sequence
taattgtatcggtttattttttttttttagcagcgaaaagaataca

SEQ ID NO: 150 - DNA, 52 bp, Artificial sequence
  Icosahedron monomer A non-crossover vertex staple sequence
ggaacaactaaattttttttttcacctatagttttttttttaagaggctgag

SEQ ID NO: 151 - DNA, 46 bp, Artificial sequence
  Icosahedron monomer A non-crossover vertex staple sequence
agaaccgccaccctcattttttttttttttaccgttcacatgaaag

SEQ ID NO: 152 - DNA, 46 bp, Artificial sequence
  Icosahedron monomer A non-crossover vertex staple sequence
aaaaataatatcccatttttttttttttagcaaatcacaacgctaa

SEQ ID NO: 153 - DNA, 52 bp, Artificial sequence
  Icosahedron monomer A non-crossover vertex staple sequence
agacgacgacaatttttttttgcgttgaacgtttttttttcataggtctgag

SEQ ID NO: 154 - DNA, 46 bp, Artificial sequence
  Icosahedron monomer A non-crossover vertex staple sequence
accagaccggaagcaatttttttttttttttttaatgatttatcaa

SEQ ID NO: 155 - DNA, 52 bp, Artificial sequence
  Icosahedron monomer A non-crossover vertex staple sequence
agcggattgcattttttttttaggggggttttttttttttgaaaatacatac

SEQ ID NO: 156 - DNA, 52 bp, Artificial sequence
  Icosahedron monomer A non-crossover vertex staple sequence
atgttagcaaactttttttttaagagaaaattttttttttgcgtctttccag

SEQ ID NO: 157 - DNA, 46 bp, Artificial sequence
  Icosahedron monomer A non-crossover vertex staple sequence
ctgcaaggcgattaagttttttttttttaacgtacaggttgtgtac

SEQ ID NO: 158 - DNA, 52 bp, Artificial sequence
  Icosahedron monomer A non-crossover vertex staple sequence
caggctgcgcaatttttttttcgtaaattaattttttttttaccccggttga

SEQ ID NO: 159 - DNA, 46 bp, Artificial sequence
  Icosahedron monomer A non-crossover vertex staple sequence
gtaccaaaaacattatttttttttttttcaatatgatgtcaatcat

SEQ ID NO: 160 - DNA, 52 bp, Artificial sequence
  Icosahedron monomer A non-crossover vertex staple sequence
ccaataaatcattttttttttcattcaacaatttttttttatttgcacgtaa

SEQ ID NO: 161 - DNA, 46 bp, Artificial sequence
  Icosahedron monomer A non-crossover vertex staple sequence
aaacgatgctgattgctttttttttttttaacattatcatatcaaa

SEQ ID NO: 162 - DNA, 46 bp, Artificial sequence
  Icosahedron monomer A non-crossover vertex staple sequence
ctcatggaaatacctattttttttttttaatggctatagcaaatga

SEQ ID NO: 163 - DNA, 52 bp, Artificial sequence
  Icosahedron monomer A non-crossover vertex staple sequence
tagaagaactcatttttttttacaggggagctttttttttgcactaaatcgg

SEQ ID NO: 164 - DNA, 46 bp, Artificial sequence
  Icosahedron monomer A non-crossover vertex staple sequence
gtttttcttttcaccattttttttttttcgagataggaggtgccgt

SEQ ID NO: 165 - DNA, 52 bp, Artificial sequence
  Icosahedron monomer A non-crossover vertex staple sequence
aaacctgtcgtgtttttttttttcgtagtgttttttttttcactggtgtgtt

SEQ ID NO: 166 - DNA, 52 bp, Artificial sequence
  Icosahedron monomer A non-crossover vertex staple sequence
cataaacatccctttttttttgtcgggttattttttttttatctaaagcatc

SEQ ID NO: 167 - DNA, 52 bp, Artificial sequence
  Icosahedron monomer A crossover vertex staple sequence
gttgggaagaaaatcacctttttactttaatcttttcgaggtgaatttctta

SEQ ID NO: 168 - DNA, 52 bp, Artificial sequence
  Icosahedron monomer A crossover vertex staple sequence
attgcgaatttttgcgggttttgtatccgtaatttttcagagccgccaccag

SEQ ID NO: 169 - DNA, 38 bp, Artificial sequence
  Icosahedron monomer A crossover vertex staple sequence
taatcaaaatggaaagcgttttcacattcaactaatgc

SEQ ID NO: 170 - DNA, 52 bp, Artificial sequence
  Icosahedron monomer A crossover vertex staple sequence
acaacatgttcattaccgttttttcaacctgtttttcaggattagagagtac

SEQ ID NO: 171 - DNA, 32 bp, Artificial sequence
  Icosahedron monomer A crossover vertex staple sequence
aaagattaaacaatttcattttgacaaaaaag

SEQ ID NO: 172 - DNA, 30 bp, Artificial sequence
  Icosahedron monomer A crossover vertex staple sequence
ctttagagttttttgcatgtagaaaccaat

SEQ ID NO: 173 - DNA, 52 bp, Artificial sequence
  Icosahedron monomer A crossover vertex staple sequence
ttgggaaggctcaccggatttttcagcacaaattttcttttgcgggagaagc

SEQ ID NO: 174 - DNA, 52 bp, Artificial sequence
  Icosahedron monomer A crossover vertex staple sequence
ggcaaggcacggagacagttttctgatacggtttttcgcggtccgttttttc

SEQ ID NO: 175 - DNA, 38 bp, Artificial sequence
  Icosahedron monomer A crossover vertex staple sequence
gacataaaatattaatttttttagggttttcccagtca

SEQ ID NO: 176 - DNA, 52 bp, Artificial sequence
  Icosahedron monomer A crossover vertex staple sequence
tatcggcctgtggcacagttttgctggatcagttttacagctgattgccctt

SEQ ID NO: 177 - DNA, 32 bp, Artificial sequence
  Icosahedron monomer A crossover vertex staple sequence
gctgcattattataaatcttttgtgcacgggt

SEQ ID NO: 178 - DNA, 30 bp, Artificial sequence
  Icosahedron monomer A crossover vertex staple sequence
taggaatgccttttcaatcgtctgaaatgg

SEQ ID NO: 179 - DNA, 42 bp, Artificial sequence
  Icosahedron monomer A connector staple sequence
aattgtagcaatacgatatattcggtcggcgccgacaatgac

SEQ ID NO: 180 - DNA, 42 bp, Artificial sequence
  Icosahedron monomer A connector staple sequence
gcaaattaaccgaagctacagaggcttttaatgccactacga

SEQ ID NO: 181 - DNA, 42 bp, Artificial sequence
  Icosahedron monomer A connector staple sequence
aacaaccatcgcgcacgggtaaaatacggaggactaaagact

SEQ ID NO: 182 - DNA, 42 bp, Artificial sequence
  Icosahedron monomer A connector staple sequence
cgacatcccttacaccttgatattcacacagcattgacagga

SEQ ID NO: 183 - DNA, 42 bp, Artificial sequence
  Icosahedron monomer A connector staple sequence
tgctcgtcataaccatacaggagtgtaccagttaatgccccc

SEQ ID NO: 184 - DNA, 42 bp, Artificial sequence
  Icosahedron monomer A connector staple sequence
ggttgaggcaggtgagtgcccgtataaatggtaataagtttt

SEQ ID NO: 185 - DNA, 42 bp, Artificial sequence
  Icosahedron monomer A connector staple sequence
cattatcaaaatcatcatcgagaacaagtatcattccaagaa

SEQ ID NO: 186 - DNA, 42 bp, Artificial sequence
  Icosahedron monomer A connector staple sequence
tcaatagtgaatacgaacgcgaggcgttttgctattttgcac

SEQ ID NO: 187 - DNA, 42 bp, Artificial sequence
  Icosahedron monomer A connector staple sequence
cgggtattaaactctaaatcaagattagttagcgaacctccc

SEQ ID NO: 188 - DNA, 42 bp, Artificial sequence
  Icosahedron monomer A connector staple sequence
ggtccttattacgcgaaaatcctgtttgttgcagcaagcggt

SEQ ID NO: 189 - DNA, 42 bp, Artificial sequence
  Icosahedron monomer A connector staple sequence
catgattaagacttagtccactattaaaccactacgtgaacc

SEQ ID NO: 190 - DNA, 42 bp, Artificial sequence
  Icosahedron monomer A connector staple sequence
ccacgctggtttgtatcagggcgatggcgaacgtggactcca

SEQ ID NO: 191 - DNA, 42 bp, Artificial sequence
  Icosahedron monomer A connector staple sequence
cgcgatgaacggtacggcgtaaattcagagtgccaagcttac

SEQ ID NO: 192 - DNA, 42 bp, Artificial sequence
  Icosahedron monomer A connector staple sequence
aaacaagagaatccgagagatagacttttcaaacttaaattt

SEQ ID NO: 193 - DNA, 42 bp, Artificial sequence
  Icosahedron monomer A connector staple sequence
ttcatgcttcggaagcagaaacagcggactccgtggtgaagg

SEQ ID NO: 194 - DNA, 42 bp, Artificial sequence
  Icosahedron monomer A connector staple sequence
cagtattaaatcctgaattttctgtatggatctaaagttttg

SEQ ID NO: 195 - DNA, 42 bp, Artificial sequence
  Icosahedron monomer A connector staple sequence
tcctgattgtttctatagttagcgtaacggattttgctaaac

SEQ ID NO: 196 - DNA, 42 bp, Artificial sequence
  Icosahedron monomer A connector staple sequence
tgggattatacttcctcagagccaccacaccctcagaaccgc

SEQ ID NO: 197 - DNA, 42 bp, Artificial sequence
  Icosahedron monomer A connector staple sequence
ccggtaccgagctcgaaacgcaataataagcagatagccgaa

SEQ ID NO: 198 - DNA, 42 bp, Artificial sequence
  Icosahedron monomer A connector staple sequence
taattgcgttgcatttttaagaaaagtaacggaatacccaaa

SEQ ID NO: 199 - DNA, 42 bp, Artificial sequence
  Icosahedron monomer A connector staple sequence
ttgctcactgcccggaacaaagtcagaggaattaactgaaca

SEQ ID NO: 200 - DNA, 42 bp, Artificial sequence
  Icosahedron monomer A connector staple sequence
cttgtttagtatcacatcacgcaaattatttataatcagtga

SEQ ID NO: 201 - DNA, 42 bp, Artificial sequence
  Icosahedron monomer A connector staple sequence
atataaagtaccgatcctgagaagtgttaccgttgtagcaat

SEQ ID NO: 202 - DNA, 42 bp, Artificial sequence
  Icosahedron monomer A connector staple sequence
gggacaaaaggtaaagggcgcgtactatcgcttaatgcgccg

SEQ ID NO: 203 - DNA, 42 bp, Artificial sequence
  Icosahedron monomer A connector staple sequence
ttcaacatgttttatgcctaatgagtgaaacatacgagccgg

SEQ ID NO: 204 - DNA, 42 bp, Artificial sequence
  Icosahedron monomer A connector staple sequence
atattttcatttgctcacaattccacacgctaactcacatta

SEQ ID NO: 205 - DNA, 42 bp, Artificial sequence
  Icosahedron monomer A connector staple sequence
ctggggcgcgagctgccagcacgcgtgcgtcataccgggggt

SEQ ID NO: 206 - DNA, 42 bp, Artificial sequence
  Icosahedron monomer A connector staple sequence
agtggttccgaaatctccagccagctttgaggggacgacgac

SEQ ID NO: 207 - DNA, 42 bp, Artificial sequence
  Icosahedron monomer A connector staple sequence
gcgatggcccacgcgcatctgccagtttccggcaccgcttct

SEQ ID NO: 208 - DNA, 42 bp, Artificial sequence
  Icosahedron monomer A connector staple sequence
aatacgtgaaccatagctttcatcaacatctggccttcctgt

SEQ ID NO: 209 - DNA, 44 bp, Artificial sequence
  A connector sequence were used for assembly
atactcgctctcgttaccgtgtggttgcatagttttctcgtcac

SEQ ID NO: 210 - DNA, 42 bp, Artificial sequence
  A connector sequence were used for assembly
tagcaacctgcctggcaagcctacgatggacacggtaacgcc

SEQ ID NO: 211 - DNA, 72 bp, Artificial sequence
  A connector sequence were used for assembly
aggcaccatcgtaggtttcttgccaggcaccatcgtaggtttcttgccaggcaccatcgtaggtttcttgcc

SEQ ID NO: 212 - DNA, 44 bp, Artificial sequence
  A bar sequence used for assembly
agagcgagtatggagtcagatggcttacttacctatcgcgctat

SEQ ID NO: 213 - DNA, 44 bp, Artificial sequence
  A bar sequence used for assembly
ttctatacaatttgcgacttatttataccaagtctttattagac

SEQ ID NO: 214 - DNA, 44 bp, Artificial sequence
  A bar sequence used for assembly
gtgtacagaaactcactgcgtcttaagaatggaacgttgtccat

SEQ ID NO: 215 - DNA, 47 bp, Artificial sequence
  A bar sequence used for assembly
ttcataagtagtagcacctatgcggcatcgcatttgagtagataggc

SEQ ID NO: 216 - DNA, 44 bp, Artificial sequence
  A bar sequence used for assembly
acacgtatcggatgtctctgtaggttgtgcagatatagataata

SEQ ID NO: 217 - DNA, 43 bp, Artificial sequence
  A bar sequence used for assembly
cagttttttagaagcgaagctggaccatgagaagttattcccg

SEQ ID NO: 218 - DNA, 44 bp, Artificial sequence
  A bar sequence used for assembly
cgatgtcagcgaacacatgtcgatgtatttaaagtgacgagaaa

SEQ ID NO: 219 - DNA, 44 bp, Artificial sequence
  A bar sequence used for assembly
tttaaatacatcgacatgtgttggtaagtaagccatctgactcc

SEQ ID NO: 220 - DNA, 44 bp, Artificial sequence
  A bar sequence used for assembly
cgctgacatcgcgggaaataacattgtatagaaatagcgcgata

SEQ ID NO: 221 - DNA, 44 bp, Artificial sequence
  A bar sequence used for assembly
ttctcatggtccagcttcgcttacttggtataaataagtcgcaa

SEQ ID NO: 222 - DNA, 44 bp, Artificial sequence
  A bar sequence used for assembly
ctaaaaaactgtattatctatatttctgtacacgtctaataaag

SEQ ID NO: 223 - DNA, 44 bp, Artificial sequence
  A bar sequence used for assembly
tctgcacaacctacagagacattccattcttaagacgcagtgag

SEQ ID NO: 224 - DNA, 44 bp, Artificial sequence
  A bar sequence used for assembly
ccgatacgtgtgcctatctactctacttatgaaatggacaacgt

SEQ ID NO: 225 - DNA, 44 bp, Artificial sequence
  A bar sequence used for assembly
caaatgcgatgccgcataggtgcaaatgcgatgccgcataggtg

SEQ ID NO: 226 - DNA, 44 bp, Artificial sequence
  A bar sequence used for assembly
ctacttatgaaatggacaacgtccgatacgtgtgcctatctact

SEQ ID NO: 227 - DNA, 44 bp, Artificial sequence
  A bar sequence used for assembly
tccattcttaagacgcagtgagtctgcacaacctacagagacat

SEQ ID NO: 228 - DNA, 44 bp, Artificial sequence
  A bar sequence used for assembly
tttctgtacacgtctaataaagctaaaaaactgtattatctata

SEQ ID NO: 229 - DNA, 44 bp, Artificial sequence
  A bar sequence used for assembly
acttggtataaataagtcgcaattctcatggtccagcttcgctt

SEQ ID NO: 230 - DNA, 44 bp, Artificial sequence
  A bar sequence used for assembly
attgtatagaaatagcgcgatacgctgacatcgcgggaaataac

SEQ ID NO: 231 - DNA, 44 bp, Artificial sequence
  A bar sequence used for assembly
ggtaagtaagccatctgactcctttaaatacatcgacatgtgtt

SEQ ID NO: 232 - DNA, 44 bp, Artificial sequence
  A sequence used to generate a 200nm bar
tggcgatacaatgcattccgcaggtaagtaagccatctgactcc

SEQ ID NO: 233 - DNA, 22 bp, Artificial sequence
  A sequence used to generate a 200nm bar
cgctgacatcggttctaatgcc

SEQ ID NO: 234 - DNA, 44 bp, Artificial sequence
  A sequence used to generate a 200nm bar
agagcgagtattgcggaatgcattgtatcgccaggcattagaac

SEQ ID NO: 235 - DNA, 57 bp, Artificial sequence
  A sequence used to construct the hemispheres
acccccattgtgttgcgttaccgtgtggttgcatagttttttatgcatatcacttct

SEQ ID NO: 236 - DNA, 16 bp, Artificial sequence
  A sequence used to construct the hemispheres
ctagcaatatctatgt

SEQ ID NO: 237 - DNA, 52 bp, Artificial sequence
  A sequence used to construct the hemispheres
actacgtccccccatcgtacgatcaggcgaggactctgaccagtctccaaca

SEQ ID NO: 238 - DNA, 27 bp, Artificial sequence
  A sequence used to construct the hemispheres
caacacaatgggggtacatagatattg

SEQ ID NO: 239 - DNA, 57 bp, Artificial sequence
  A sequence used to construct the hemispheres
ccacgagttccttatccgtagggctatattgacactgtttttagagatgaaatctgt

SEQ ID NO: 240 - DNA, 57 bp, Artificial sequence; identical to SEQ ID NO: 239
  A sequence used to construct the hemispheres

SEQ ID NO: 241 - DNA, 27 bp, Artificial sequence
  A sequence used to construct the hemispheres
ataaggaactcgtggacatagatattg

SEQ ID NO: 242 - DNA, 52 bp, Artificial sequence
  A sequence used to construct the hemispheres
tgcttcgtgtgcgacagagcttatccattggctgggagcgggtcattgaccc

SEQ ID NO: 243 - DNA, 27 bp, Artificial sequence
  A sequence used to construct the hemispheres
gtcgcacacgaagcaacatagatattg

SEQ ID NO: 244 - DNA, 52 bp, Artificial sequence
  A sequence used to construct the hemispheres
gacaacaaacctgaaagagcttatccattggctgggaggtgttgctcctgcc

SEQ ID NO: 245 - DNA, 27 bp, Artificial sequence
  A sequence used to construct the hemispheres
gcggttgcgtgaacgcaatatctatgt

SEQ ID NO: 246 - DNA, 16 bp, Artificial sequence
  A sequence used to construct the hemispheres
catgacatagatattg

SEQ ID NO: 247 - DNA, 52 bp, Artificial sequence; identical to SEQ ID NO: 244
  A sequence used to construct the hemispheres

SEQ ID NO: 248 - DNA, 27 bp, Artificial sequence
  A sequence used to construct the hemispheres
ttcaggtttgttgtccaatatctatgt

SEQ ID NO: 249 - DNA, 52 bp, Artificial sequence
  A sequence used to construct the hemispheres
tgctgcaggcgcaagcgtacgatcaggcgaggactctcccaacacataaaga

SEQ ID NO: 250 - DNA, 16 bp, Artificial sequence
  A sequence used to construct the hemispheres
ctagacatagatattg

SEQ ID NO: 251 - DNA, 27 bp, Artificial sequence
  A sequence used to construct the hemispheres
cttgcgcctgcagcacaatatctatgt

SEQ ID NO: 252 - DNA, 57 bp, Artificial sequence
  A sequence used to construct the hemispheres
tatttcggcgttaacccgtagggctatattgacactgttttttgtcagggcgtagca

SEQ ID NO: 253 - DNA, 57 bp, Artificial sequence
  A sequence used to construct the hemispheres
atcgttgtatgctagccgtagggctatattgacactgtttttatagactagaacctt

SEQ ID NO: 254 - DNA, 57 bp, Artificial sequence
  A sequence used to construct the hemispheres
agccatgattacattcgttaccgtgtggttgcatagttttttgttacgcgcaaattt

SEQ ID NO: 255 - DNA, 27 bp, Artificial sequence
  A sequence used to construct the hemispheres
aatgtaatcatggctcaatatctatgt

SEQ ID NO: 256 - DNA, 52 bp, Artificial sequence
  A sequence used to construct the hemispheres
tcacgcgaagaggctagagcttatccattggctgggaccgacatgctaaaag

SEQ ID NO: 257 - DNA, 27 bp, Artificial sequence
  A sequence used to construct the hemispheres
agcctcttcgcgtgacaatatctatgt

SEQ ID NO: 258 - DNA, 52 bp, Artificial sequence
  A sequence used to construct the hemispheres
ctgcgacgctgaggccgtacgatcaggcgaggactcttaacaggatgcaaat

SEQ ID NO: 259 - DNA, 16 bp, Artificial sequence
  A sequence used to construct the hemispheres
ctagacatagatattg

SEQ ID NO: 260 - DNA, 27 bp, Artificial sequence
  A sequence used to construct the hemispheres
gcctcagcgtcgcagcaatatctatgt

SEQ ID NO: 261 - DNA, 57 bp, Artificial sequence
  A sequence used to construct the hemispheres
atcggcattacgcttccgtagggctatattgacactgtttttatcggcattacgctt

SEQ ID NO: 262 - DNA, 57 bp, Artificial sequence
  A sequence used to construct the hemispheres
aagcgtaatgccgatccgtagggctatattgacactgttttttataacgggacaacg

SEQ ID NO: 263 - DNA, 57 bp, Artificial sequence
  A sequence used to construct the hemispheres
tcggggagtacctctcgttaccgtgtggttgcatagttttttgtttttattatagaa

SEQ ID NO: 264 - DNA, 27 bp, Artificial sequence
  A sequence used to construct the hemispheres
agaggtactccccgacaatatctatgt

SEQ ID NO: 265 - DNA, 52 bp, Artificial sequence
  A sequence used to construct the hemispheres
gcggcttgattatatcgtacgatcaggcgaggactctccgacgggcacatac

SEQ ID NO: 266 - DNA, 27 bp, Artificial sequence
  A sequence used to construct the hemispheres
atataatcaagccgccaatatctatgt

SEQ ID NO: 267 - DNA, 52 bp, Artificial sequence
  A sequence used to construct the hemispheres
attccgcatgagcgaagagcttatccattggctgggatttggagcagtccca

SEQ ID NO: 268 - DNA, 27 bp, Artificial sequence
  A sequence used to construct the hemispheres
tcgctcatgcggaatcaatatctatgt

SEQ ID NO: 269 - DNA, 57 bp, Artificial sequence
  A sequence used to construct the hemispheres
ctagcatacaacgatccgtagggctatattgacactgtttttggtcgcatgcatcgt

SEQ ID NO: 270 - DNA, 57 bp, Artificial sequence
  A sequence used to construct the hemispheres
gttaacgccgaaataccgtagggctatattgacactgttttttgttcagctcctccg

SEQ ID NO: 271 - DNA, 57 bp, Artificial sequence
  A sequence used to construct the hemispheres
caagtctgtcgataacgttaccgtgtggttgcatagtttttttctgtagagcggaat

SEQ ID NO: 272 - DNA, 27 bp, Artificial sequence
  A sequence used to construct the hemispheres
ttatcgacagacttgcaatatctatgt

SEQ ID NO: 273 - DNA, 52 bp, Artificial sequence
  A sequence used to construct the hemispheres
agtggcgcttgcttacgtacgatcaggcgaggactcttctggccatcgataa

SEQ ID NO: 274 - DNA, 27 bp, Artificial sequence
  A sequence used to construct the hemispheres
taagcaagcgccactcaatatctatgt

SEQ ID NO: 275 - DNA, 52 bp, Artificial sequence
  A sequence used to construct the hemispheres
agacgccgtgttgacagagcttatccattggctgggaaaaggcgacagcatt

SEQ ID NO: 276 - DNA, 27 bp, Artificial sequence
  A sequence used to construct the hemispheres
gtcaacacggcgtctcaatatctatgt

SEQ ID NO: 277 - DNA, 57 bp, Artificial sequence
  A sequence used to construct the hemispheres
gagtgaagtaagagcccgtagggctatattgacactgttttttggcgcacataacta

SEQ ID NO: 278 - DNA, 57 bp, Artificial sequence
  A sequence used to construct the hemispheres
gctcttacttcactcccgtagggctatattgacactgttttttgttcattttggaat

SEQ ID NO: 279 - DNA, 48 bp, Artificial sequence
  A sequence used to construct the hemispheres
aaaaaactatgcaacctgcctggcaagcctacgatggggataagctct

SEQ ID NO: 280 - DNA, 43 bp, Artificial sequence
  A sequence used to construct the hemispheres
tcccagccaattgcctggcaagcctacgatggtagccctacgg

SEQ ID NO: 281 - DNA, 48 bp, Artificial sequence
  A sequence used to construct the hemispheres
aaaaacagtgtcaatatgcctggcaagcctacgatggctgatcgtacg

SEQ ID NO: 282 - DNA, 43 bp, Artificial sequence
  A sequence used to construct the hemispheres
agagtcctcgctgcctggcaagcctacgatggacacggtaacg

SEQ ID NO: 283 - DNA, 96 bp, Artificial sequence
  A sequence used to construct the hemispheres
aggcaccatcgtaggtttcttgccaggcaccatcgtaggtttcttgccaggcaccatcgtaggtttcttgccaggcaccatcgtaggtttcttgcc

SEQ ID NO: 284 - DNA, 30 bp, Artificial sequence
  DNA mesh1
cgctatacgtgttcaccgcttgctagcagt

SEQ ID NO: 285 - DNA, 30 bp, Artificial sequence
  DNA mesh2
cgctatagcaagcggactctggccttcgat

SEQ ID NO: 286 - DNA, 30 bp, Artificial sequence
  DNA mesh3
cgctatagccagagtggaaggcgaggatca

SEQ ID NO: 287 - DNA, 30 bp, Artificial sequence
  DNA mesh4
cgctatacgccttcctgaacacggttacag

SEQ ID NO: 288 - DNA, 10 bp, Artificial sequence
  DNA sequence that is modified to have an amino group at their 5′ end to facilitate easy binding of active agents
tttctgtaac

SEQ ID NO: 289 - DNA, 10 bp, Artificial sequence
  DNA sequence that is modified to have an amino group at their 5′ end to facilitate easy binding of active agents
tttactgcta

SEQ ID NO: 290 - DNA, 10 bp, Artificial sequence
  DNA sequence that is modified to have an amino group at their 5′ end to facilitate easy binding of active agents
tttatcgaag

SEQ ID NO: 291 - DNA, 10 bp, Artificial sequence
  DNA sequence that is modified to have an amino group at their 5′ end to facilitate easy binding of active agents
ttttgatcct

SEQ ID NO: 292 - DNA, 28 bp, Artificial sequence
  A DNA staple used to construct the cube
ccaacgtcaattccagttcccttaagca

SEQ ID NO: 293 - DNA, 49 bp, Artificial sequence
  A DNA staple used to construct the cube
ggttccgaaatcggcaaaattggaacagggatacctcagagccaccacc

SEQ ID NO: 294 - DNA, 21 bp, Artificial sequence
  A DNA staple used to construct the cube
gtccacgctggtgttagcgta

SEQ ID NO: 295 - DNA, 21 bp, Artificial sequence
  A DNA staple used to construct the cube
ggctggccctcttttcagcgc

SEQ ID NO: 296 - DNA, 49 bp, Artificial sequence
  A DNA staple used to construct the cube
ctgattgcccttcaccgccgaaaatcattccaccagtacaaactacaac

SEQ ID NO: 297 - DNA, 21 bp, Artificial sequence
  A DNA staple used to construct the cube
gggcgccagggtactttcaac

SEQ ID NO: 298 - DNA, 21 bp, Artificial sequence
  A DNA staple used to construct the cube
gggagtcgggtgagctatacg

SEQ ID NO: 299 - DNA, 49 bp, Artificial sequence
  A DNA staple used to construct the cube
cattaatgaatcggccaacccagtgatgtatggtccagacgttagtaaa

SEQ ID NO: 300 - DNA, 21 bp, Artificial sequence
  A DNA staple used to construct the cube
cgctcactgcccaatctccaa

SEQ ID NO: 301 - DNA, 49 bp, Artificial sequence
  A DNA staple used to construct the cube
gcctggggtgcctaatgagaaacctgtaataataaggaacaactaaagg

SEQ ID NO: 302 - DNA, 21 bp, Artificial sequence
  A DNA staple used to construct the cube
ctcacaattccaacagcttga

SEQ ID NO: 303 - DNA, 21 bp, Artificial sequence
  A DNA staple used to construct the cube
agctgtttccaggatccaacg

SEQ ID NO: 304 - DNA, 49 bp, Artificial sequence
  A DNA staple used to construct the cube
tcgtaatcatggtcatagccggaagcctttcgattaattgtatcggttt

SEQ ID NO: 305 - DNA, 21 bp, Artificial sequence
  A DNA staple used to construct the cube
gcctgcaggtcgcttgcaggg

SEQ ID NO: 306 - DNA, 21 bp, Artificial sequence
  A DNA staple used to construct the cube
acgattaagtttcgctaaggt

SEQ ID NO: 307 - DNA, 49 bp, Artificial sequence
  A DNA staple used to construct the cube
cccagtcacgacgttgtaaccgggtacgatataacaacaaccatcgccc

SEQ ID NO: 308 - DNA, 21 bp, Artificial sequence
  A DNA staple used to construct the cube
ggggatgtgctgcggctacag

SEQ ID NO: 309 - DNA, 36 bp, Artificial sequence
  A DNA staple used to construct the cube
ggcctctgggtaagcatcggtcgtcaccctcagcag

SEQ ID NO: 310 - DNA, 32 bp, Artificial sequence
  A DNA staple used to construct the cube
ggaagggcgagcgcatcatgtgagtgtataag

SEQ ID NO: 311 - DNA, 31 bp, Artificial sequence
  A DNA staple used to construct the cube
attcaggctggccagttgccagcttgtaaac

SEQ ID NO: 312 - DNA, 32 bp, Artificial sequence
  A DNA staple used to construct the cube
accaggcaaagcagtatcttcgcgtaaaattc

SEQ ID NO: 313 - DNA, 37 bp, Artificial sequence
  A DNA staple used to construct the cube
ttccggcaccgcttctcgcactctaggaacttaaatc

SEQ ID NO: 314 - DNA, 21 bp, Artificial sequence
  A DNA staple used to construct the cube
ggattgaccgtactccatgtt

SEQ ID NO: 315 - DNA, 21 bp, Artificial sequence
  A DNA staple used to construct the cube
cactcggattaagccccaaaa

SEQ ID NO: 316 - DNA, 18 bp, Artificial sequence
  A DNA staple used to construct the cube
caacccggttggtgtgtg

SEQ ID NO: 317 - DNA, 18 bp, Artificial sequence
  A DNA staple used to construct the cube
tcctgtatgagggggaaa

SEQ ID NO: 318 - DNA, 31 bp, Artificial sequence
  A DNA staple used to construct the cube
tgataatcagaactccgtgggaacaaacggc

SEQ ID NO: 319 - DNA, 36 bp, Artificial sequence
  A DNA staple used to construct the cube
acaagcatgtcggtcaaaccagaaattaccttatgc

SEQ ID NO: 320 - DNA, 28 bp, Artificial sequence
  A DNA staple used to construct the cube
caaataatgaacgaactgacaggcttgc

SEQ ID NO: 321 - DNA, 42 bp, Artificial sequence
  A DNA staple used to construct the cube
aatttcatcaacattaagtaaccgttgtatcaacgggtaaaa

SEQ ID NO: 322 - DNA, 49 bp, Artificial sequence
  A DNA staple used to construct the cube
gttaatactggagcgacagataagctgctcattcaaaagaattatttca

SEQ ID NO: 323 - DNA, 39 bp, Artificial sequence
  A DNA staple used to construct the cube
gcattaaatcaggtccaggcgattacccaaatcaaaata

SEQ ID NO: 324 - DNA, 45 bp, Artificial sequence
  A DNA staple used to construct the cube
gctatttttggccatcaaaaataaggcctcacccagcgaaacgaa

SEQ ID NO: 325 - DNA, 24 bp, Artificial sequence
  A DNA staple used to construct the cube
taatgtgtaggtatgtaccccggt

SEQ ID NO: 326 - DNA, 40 bp, Artificial sequence
  A DNA staple used to construct the cube
caaaaggcatatattagcattttggggcataaaacgaact

SEQ ID NO: 327 - DNA, 21 bp, Artificial sequence
  A DNA staple used to construct the cube
gacagtgataaaacatacagg

SEQ ID NO: 328 - DNA, 22 bp, Artificial sequence
  A DNA staple used to construct the cube
tatgataaagcctttagcaaaa

SEQ ID NO: 329 - DNA, 24 bp, Artificial sequence
  A DNA staple used to construct the cube
acctgagagtttttgttctggcct

SEQ ID NO: 330 - DNA, 34 bp, Artificial sequence
  A DNA staple used to construct the cube
aaccctgtgagaataaaactggaagatcgagtaa

SEQ ID NO: 331 - DNA, 25 bp, Artificial sequence
  A DNA staple used to construct the cube
acgcaagcaaatcaagaatcgttta

SEQ ID NO: 332 - DNA, 30 bp, Artificial sequence
  A DNA staple used to construct the cube
gtaccaaaaacattatgacattaatgaaag

SEQ ID NO: 333 - DNA, 23 bp, Artificial sequence
  A DNA staple used to construct the cube
tcaattctactataaattggaac

SEQ ID NO: 334 - DNA, 22 bp, Artificial sequence
  A DNA staple used to construct the cube
tagtttaaatgcaatgcctgag

SEQ ID NO: 335 - DNA, 45 bp, Artificial sequence
  A DNA staple used to construct the cube
caaggcgtgaatacaactttcggagattgcatctcgcaactgttg

SEQ ID NO: 336 - DNA, 45 bp, Artificial sequence
  A DNA staple used to construct the cube
ttaagccgtaacagaacggtcaagcgcacgacgacgccattcgcc

SEQ ID NO: 337 - DNA, 21 bp, Artificial sequence
  A DNA staple used to construct the cube
aagtacttttgcgggagttca

SEQ ID NO: 338 - DNA, 44 bp, Artificial sequence
  A DNA staple used to construct the cube
cataaagatattccataggctttgaccggaagatggtgccggaa

SEQ ID NO: 339 - DNA, 37 bp, Artificial sequence
  A DNA staple used to construct the cube
ctatattttcataacatccgagaaactcataaggata

SEQ ID NO: 340 - DNA, 24 bp, Artificial sequence
  A DNA staple used to construct the cube
attagatacattgaaaaggtggca

SEQ ID NO: 341 - DNA, 29 bp, Artificial sequence
  A DNA staple used to construct the cube
aacccatataatgtttttaccagacgacg

SEQ ID NO: 342 - DNA, 21 bp, Artificial sequence
  A DNA staple used to construct the cube
tcccaattctaacctgtttag

SEQ ID NO: 343 - DNA, 23 bp, Artificial sequence
  A DNA staple used to construct the cube
ggtgtctggaagaggtagaaaaa

SEQ ID NO: 344 - DNA, 22 bp, Artificial sequence
  A DNA staple used to construct the cube
attgagtagatttagtttgacc

SEQ ID NO: 345 - DNA, 11 bp, Artificial sequence
  A DNA staple used to construct the cube
acacagttgat

SEQ ID NO: 346 - DNA, 24 bp, Artificial sequence
  A DNA staple used to construct the cube
taattgctgaatcaactaaagtac

SEQ ID NO: 347 - DNA, 12 bp, Artificial sequence
  A DNA staple used to construct the cube
tggcttagagct

SEQ ID NO: 348 - DNA, 29 bp, Artificial sequence
  A DNA staple used to construct the cube
tttctttaataaccagataaacagttcag

SEQ ID NO: 349 - DNA, 11 bp, Artificial sequence
  A DNA staple used to construct the cube
tgataagagca

SEQ ID NO: 350 - DNA, 23 bp, Artificial sequence
  A DNA staple used to construct the cube
ggtcaggattaggaggctttcat

SEQ ID NO: 351 - DNA, 11 bp, Artificial sequence
  A DNA staple used to construct the cube
cgtgctccttt

SEQ ID NO: 352 - DNA, 24 bp, Artificial sequence
  A DNA staple used to construct the cube
atatcgcgttttcaaactccaaca

SEQ ID NO: 353 - DNA, 29 bp, Artificial sequence
  A DNA staple used to construct the cube
cttcaattaagagagcaaagcggattgca

SEQ ID NO: 354 - DNA, 45 bp, Artificial sequence
  A DNA staple used to construct the cube
aaaacgagaatgaccataaagtcagagaagcccgaaagacttcaa

SEQ ID NO: 355 - DNA, 38 bp, Artificial sequence
  A DNA staple used to construct the cube
gtcgggtaatagtaaaaatagcgaagagtacttgcgga

SEQ ID NO: 356 - DNA, 41 bp, Artificial sequence
  A DNA staple used to construct the cube
atagcgtccaatactgcggaatgcttccggaagaattcgag

SEQ ID NO: 357 - DNA, 21 bp, Artificial sequence
  A DNA staple used to construct the cube
ttttgccagaggataaatatt

SEQ ID NO: 358 - DNA, 24 bp, Artificial sequence
  A DNA staple used to construct the cube
ataaaaaccaaatgtttagactgg

SEQ ID NO: 359 - DNA, 21 bp, Artificial sequence
  A DNA staple used to construct the cube
gcaacactattgcaaaagaag

SEQ ID NO: 360 - DNA, 35 bp, Artificial sequence
  A DNA staple used to construct the cube
aacacgaggcaataccacattcaattattactttc

SEQ ID NO: 361 - DNA, 28 bp, Artificial sequence
  A DNA staple used to construct the cube
gttaaatatgataatgctgtagctgtca

SEQ ID NO: 362 - DNA, 23 bp, Artificial sequence
  A DNA staple used to construct the cube
cataacgccaaaaggaattcctc

SEQ ID NO: 363 - DNA, 21 bp, Artificial sequence
  A DNA staple used to construct the cube
ttgagatttaggatagtaaga

SEQ ID NO: 364 - DNA, 24 bp, Artificial sequence
  A DNA staple used to construct the cube
aacggaacaacactaatgcagata

SEQ ID NO: 365 - DNA, 21 bp, Artificial sequence
  A DNA staple used to construct the cube
gttgggaagaagattcatcag

SEQ ID NO: 366 - DNA, 35 bp, Artificial sequence
  A DNA staple used to construct the cube
atcttatacctttaatcattgtgacgagtagatag

SEQ ID NO: 367 - DNA, 28 bp, Artificial sequence
  A DNA staple used to construct the cube
ttagcgagcttcgcaaatggtcaatgcg

SEQ ID NO: 368 - DNA, 23 bp, Artificial sequence
  A DNA staple used to construct the cube
gattttaagaactggctcatacg

SEQ ID NO: 369 - DNA, 21 bp, Artificial sequence
  A DNA staple used to construct the cube
tttaatttcaacagtcaggac

SEQ ID NO: 370 - DNA, 21 bp, Artificial sequence
  A DNA staple used to construct the cube
cctgacaataaatatttttag

SEQ ID NO: 371 - DNA, 31 bp, Artificial sequence
  A DNA staple used to construct the cube
gtaatcttgacaagaaccggctaaatcggtt

SEQ ID NO: 372 - DNA, 21 bp, Artificial sequence
  A DNA staple used to construct the cube
acttagccgggcttgagatgg

SEQ ID NO: 373 - DNA, 36 bp, Artificial sequence
  A DNA staple used to construct the cube
gagcgacctgatgggatttacgccagctggcgaaag

SEQ ID NO: 374 - DNA, 39 bp, Artificial sequence
  A DNA staple used to construct the cube
agacattgccgttctagctgataacctgtaacctcagag

SEQ ID NO: 375 - DNA, 24 bp, Artificial sequence
  A DNA staple used to construct the cube
ccggcgcagacaatcataaagatt

SEQ ID NO: 376 - DNA, 36 bp, Artificial sequence
  A DNA staple used to construct the cube
tcgttcatgaggaagttaaagacacgccagggtttt

SEQ ID NO: 377 - DNA, 18 bp, Artificial sequence
  A DNA staple used to construct the cube
aattagatggtcggtgcg

SEQ ID NO: 378 - DNA, 35 bp, Artificial sequence
  A DNA staple used to construct the cube
caacactacgaaggcaccaacctaattatacgtac

SEQ ID NO: 379 - DNA, 42 bp, Artificial sequence
  A DNA staple used to construct the cube
aaaacactcatctggctgagatctacccggagagggtagcta

SEQ ID NO: 380 - DNA, 49 bp, Artificial sequence
  A DNA staple used to construct the cube
aggctttgaggactaagtagcaacaaggcggccagtgccaagcttgcat

SEQ ID NO: 381 - DNA, 38 bp, Artificial sequence
  A DNA staple used to construct the cube
tacgtaatgcagtacaagaaagagaaacaagccatcaa

SEQ ID NO: 382 - DNA, 19 bp, Artificial sequence
  A DNA staple used to construct the cube
agaggcaaaagaatacact

SEQ ID NO: 383 - DNA, 38 bp, Artificial sequence
  A DNA staple used to construct the cube
cgtccattaatcgcctggaaccggtaatcgaggccgga

SEQ ID NO: 384 - DNA, 49 bp, Artificial sequence
  A DNA staple used to construct the cube
agttaaaggccgctttgctgaggactctagtgtgtgaaattgttatccg

SEQ ID NO: 385 - DNA, 21 bp, Artificial sequence
  A DNA staple used to construct the cube
ggaaacgaggagactttaaat

SEQ ID NO: 386 - DNA, 21 bp, Artificial sequence
  A DNA staple used to construct the cube
acgcataacccgagctcgaat

SEQ ID NO: 387 - DNA, 49 bp, Artificial sequence
  A DNA staple used to construct the cube
taccgatagttgcgccttcttaacacaacaactcacattaattgcgttg

SEQ ID NO: 388 - DNA, 21 bp, Artificial sequence
  A DNA staple used to construct the cube
aatgacaatgttcggtctgcg

SEQ ID NO: 389 - DNA, 21 bp, Artificial sequence
  A DNA staple used to construct the cube
atcagcttgataaagtgtaaa

SEQ ID NO: 390 - DNA, 49 bp, Artificial sequence
  A DNA staple used to construct the cube
aaaaaaggctccaaaacgttgaagctttccgagaggcggtttgcgtatt

SEQ ID NO: 391 - DNA, 21 bp, Artificial sequence
  A DNA staple used to construct the cube
aattgcgaatcgtgccagctg

SEQ ID NO: 392 - DNA, 49 bp, Artificial sequence
  A DNA staple used to construct the cube
agtttcagcggagtgactaaacaggtttttgagagagttgcagcaagcg

SEQ ID NO: 393 - DNA, 21 bp, Artificial sequence
  A DNA staple used to construct the cube
agatttttcaggagcctggtg

SEQ ID NO: 394 - DNA, 21 bp, Artificial sequence
  A DNA staple used to construct the cube
tgaattttcgacgggcaacag

SEQ ID NO: 395 - DNA, 49 bp, Artificial sequence
  A DNA staple used to construct the cube
acgatctaaagttttgcctcatattgcccctaaatcaaaagaatagccc

SEQ ID NO: 396 - DNA, 21 bp, Artificial sequence
  A DNA staple used to construct the cube
agctcgtcttgattttggaat

SEQ ID NO: 397 - DNA, 21 bp, Artificial sequence
  A DNA staple used to construct the cube
gcctgtagcctgtttgatggt

SEQ ID NO: 398 - DNA, 41 bp, Artificial sequence
  A DNA staple used to construct the cube
accgtaacactgagttcaataggagtgttgagggcgaaaaa

SEQ ID NO: 399 - DNA, 52 bp, Artificial sequence
  A DNA staple used to construct the cube
acaagtcaccttgctttcgctgagatagggttgaacccatcgccaccctcag

SEQ ID NO: 400 - DNA, 21 bp, Artificial sequence
  A DNA staple used to construct the cube
ctcattttcaagagtccacta

SEQ ID NO: 401 - DNA, 18 bp, Artificial sequence
  A DNA staple used to construct the cube
ttttttttctcagaacgt

SEQ ID NO: 402 - DNA, 27 bp, Artificial sequence
  A DNA staple used to construct the cube
aaccgccacgcaagcctcgtcacagac

SEQ ID NO: 403 - DNA, 39 bp, Artificial sequence
  A DNA staple used to construct the cube
cattgaatcatcaggtctcgcaagaaagtcgggacgagt

SEQ ID NO: 404 - DNA, 32 bp, Artificial sequence
  A DNA staple used to construct the cube
catattctgtgtaaaccagagtcaaaaagaag

SEQ ID NO: 405 - DNA, 55 bp, Artificial sequence
  A DNA staple used to construct the cube
agcgacgatctcggacccgcattaccctgactattatatcaaaacccctcaaatc

SEQ ID NO: 406 - DNA, 39 bp, Artificial sequence
  A DNA staple used to construct the cube
cattgaatcatcaggtctcacggcaagataactgcggat

SEQ ID NO: 407 - DNA, 32 bp, Artificial sequence
  A DNA staple used to construct the cube
ccttcccaccgcctagcgagttcaaaaagaag

SEQ ID NO: 408 - DNA, 55 bp, Artificial sequence
  A DNA staple used to construct the cube
gtctaggaagtcttctacctgttaccctgactattatatcaaaacccctcaaatc

SEQ ID NO: 409 - DNA, 39 bp, Artificial sequence
  A DNA staple used to construct the cube
cattgaatcatcaggtcttactatgggagacgtttctcc

SEQ ID NO: 410 - DNA, 32 bp, Artificial sequence
  A DNA staple used to construct the cube
tatagggccggcttcatagagtcaaaaagaag

SEQ ID NO: 411 - DNA, 55 bp, Artificial sequence
  A DNA staple used to construct the cube
cctgactgaaagttactctttttaccctgactattatatcaaaacccctcaaatc

SEQ ID NO: 412 - DNA, 39 bp, Artificial sequence
  A DNA staple used to construct the cube
cattgaatcatcaggtcttttcttgtggactctagacct

SEQ ID NO: 413 - DNA, 32 bp, Artificial sequence
  A DNA staple used to construct the cube
actttagacgccccacctttatcaaaaagaag

SEQ ID NO: 414 - DNA, 55 bp, Artificial sequence
  A DNA staple used to construct the cube
caacgcagccacgaagcactattaccctgactattatatcaaaacccctcaaatc

SEQ ID NO: 415 - DNA, 39 bp, Artificial sequence
  A DNA staple used to construct the cube
cattgaatcatcaggtcttatccctcggtcagagaaact

SEQ ID NO: 416 - DNA, 32 bp, Artificial sequence
  A DNA staple used to construct the cube
gatatggttctattacgctcatcaaaaagaag

SEQ ID NO: 417 - DNA, 55 bp, Artificial sequence
  A DNA staple used to construct the cube
gcgtctacggtaaacataagcttaccctgactattatatcaaaacccctcaaatc

SEQ ID NO: 418 - DNA, 39 bp, Artificial sequence
  A DNA staple used to construct the cube
cattgaatcatcaggtcttcatgctatcacaccatgtcg

SEQ ID NO: 419 - DNA, 32 bp, Artificial sequence
  A DNA staple used to construct the cube
gtcgggcagaaatatggatcgtcaaaaagaag

SEQ ID NO: 420 - DNA, 55 bp, Artificial sequence
  A DNA staple used to construct the cube
acttcgtggtgactgacggtattaccctgactattatatcaaaacccctcaaatc

SEQ ID NO: 421 - DNA, 39 bp, Artificial sequence
  A DNA staple used to construct the cube
cattgaatcatcaggtcttgaggatgtagtgttgctcac

SEQ ID NO: 422 - DNA, 32 bp, Artificial sequence
  A DNA staple used to construct the cube
ctccgtgattcctatagcaggtcaaaaagaag

SEQ ID NO: 423 - DNA, 55 bp, Artificial sequence
  A DNA staple used to construct the cube
agcgaaagcaaggtgacttgtttaccctgactattatatcaaaacccctcaaatc

SEQ ID NO: 424 - DNA, 39 bp, Artificial sequence
  A DNA staple used to construct the cube
cattgaatcatcaggtctatctccgccgaaaagttcagt

SEQ ID NO: 425 - DNA, 32 bp, Artificial sequence
  A DNA staple used to construct the cube
acaccctgggcaagtgccagctcaaaaagaag

SEQ ID NO: 426 - DNA, 55 bp, Artificial sequence
  A DNA staple used to construct the cube
gctcgaattcgaaccattcctttaccctgactattatatcaaaacccctcaaatc

SEQ ID NO: 427 - DNA, 30 bp, Artificial sequence
  A DNA staple used to construct the cube
agctcatttgtcgctggggtgctggcgtcc

SEQ ID NO: 428 - DNA, 39 bp, Artificial sequence
  A DNA staple used to construct the cube
gttacgtctatctggggaggctttaaccaacagccagct

SEQ ID NO: 429 - DNA, 42 bp, Artificial sequence
  A DNA staple used to construct the cube
ggtagtcgcgaacacgcgaagtttttgagaccttcatcaaga

SEQ ID NO: 430 - DNA, 36 bp, Artificial sequence
  A DNA staple used to construct the cube
agctcatttgtatttgtatttgaggaaaggccctgc

SEQ ID NO: 431 - DNA, 39 bp, Artificial sequence
  A DNA staple used to construct the cube
attatgggtgtacggacgctatttaaccaacagccagct

SEQ ID NO: 432 - DNA, 42 bp, Artificial sequence
  A DNA staple used to construct the cube
gctcaacctgcttttctgctgtttttgagaccttcatcaaga

SEQ ID NO: 433 - DNA, 30 bp, Artificial sequence
  A DNA staple used to construct the cube
agctcatttgcagggcctttcctcaaatac

SEQ ID NO: 434 - DNA, 39 bp, Artificial sequence
  A DNA staple used to construct the cube
tagcgtccgtacacccataattttaaccaacagccagct

SEQ ID NO: 435 - DNA, 42 bp, Artificial sequence
  A DNA staple used to construct the cube
cagcagaaaagcaggttgagctttttgagaccttcatcaaga

SEQ ID NO: 436 - DNA, 30 bp, Artificial sequence
  A DNA staple used to construct the cube
agctcatttggacgccagcaccccagcgac

SEQ ID NO: 437 - DNA, 39 bp, Artificial sequence
  A DNA staple used to construct the cube
gcctccccagatagacgtaactttaaccaacagccagct

SEQ ID NO: 438 - DNA, 42 bp, Artificial sequence
  A DNA staple used to construct the cube
cttcgcgtgttcgcgactacctttttgagaccttcatcaaga

SEQ ID NO: 439 - DNA, 30 bp, Artificial sequence
  A DNA staple used to construct the cube
agctcatttgttgacggcgttctcacagaa

SEQ ID NO: 440 - DNA, 39 bp, Artificial sequence
  A DNA staple used to construct the cube
gatatttctcattttcttcactttaaccaacagccagct

SEQ ID NO: 441 - DNA, 42 bp, Artificial sequence
  A DNA staple used to construct the cube
ctacatgtactgataagtgcttttttgagaccttcatcaaga

SEQ ID NO: 442 - DNA, 30 bp, Artificial sequence
  A DNA staple used to construct the cube
agctcatttactgcctttacaatatcaatg

SEQ ID NO: 443 - DNA, 39 bp, Artificial sequence; identical to SEQ ID NO: 440
  A DNA staple used to construct the cube

SEQ ID NO: 444 - DNA, 42 bp, Artificial sequence
  A DNA staple used to construct the cube
cctcaaatgctcgcacttttatttttgagaccttcatcaaga

SEQ ID NO: 445 - DNA, 30 bp, Artificial sequence
  A DNA staple used to construct the cube
agctcatttcattgatattgtaaaggcagt

SEQ ID NO: 446 - DNA, 39 bp, Artificial sequence
  A DNA staple used to construct the cube
gtgaagaaaatgagaaatatctttaaccaacagccagct

SEQ ID NO: 447 - DNA, 42 bp, Artificial sequence
  A DNA staple used to construct the cube
taaaagtgcgagcatttgaggtttttgagaccttcatcaaga

SEQ ID NO: 448 - DNA, 30 bp, Artificial sequence
  A DNA staple used to construct the cube
agctcatttttctgtgagaacgccgtcaac

SEQ ID NO: 449 - DNA, 39 bp, Artificial sequence
  A DNA staple used to construct the cube
ataattaactgggtacctctatttaaccaacagccagct

SEQ ID NO: 450 - DNA, 42 bp, Artificial sequence
  A DNA staple used to construct the cube
agcacttatcagtacatgtagtttttgagaccttcatcaaga

SEQ ID NO: 451 - DNA, 31 bp, Artificial sequence
  A DNA staple used to construct the cube
ccgtctatcaatccgcagttatcttgccgtg

SEQ ID NO: 452 - DNA, 37 bp, Artificial sequence
  A DNA staple used to construct the cube
actcgctaggcggtgggaaggttaaagaacgtggact

SEQ ID NO: 453 - DNA, 52 bp, Artificial sequence
  A DNA staple used to construct the cube
caggtagaagacttcctagacgagatagggttgaacccatcgccaccctcag

SEQ ID NO: 454 - DNA, 31 bp, Artificial sequence
  A DNA staple used to construct the cube
ccgtctatcacgacatggtgtgatagcatga

SEQ ID NO: 455 - DNA, 37 bp, Artificial sequence
  A DNA staple used to construct the cube
cgatccatatttctgcccgacttaaagaacgtggact

SEQ ID NO: 456 - DNA, 53 bp, Artificial sequence
  A DNA staple used to construct the cube
taccgtcagtcaccacgaagtcgagatagggttgaacccatcgccaccctcag

SEQ ID NO: 457 - DNA, 31 bp, Artificial sequence
  A DNA staple used to construct the cube
ccgtctatcaaggtctagagtccacaagaaa

SEQ ID NO: 458 - DNA, 37 bp, Artificial sequence
  A DNA staple used to construct the cube
taaaggtggggcgtctaaagtttaaagaacgtggact

SEQ ID NO: 459 - DNA, 52 bp, Artificial sequence
  A DNA staple used to construct the cube
tagtgcttcgtggctgcgttggagatagggttgaacccatcgccaccctcag

SEQ ID NO: 460 - DNA, 31 bp, Artificial sequence
  A DNA staple used to construct the cube
ccgtctatcaactgaacttttcggcggagat

SEQ ID NO: 461 - DNA, 37 bp, Artificial sequence
  A DNA staple used to construct the cube
gctggcacttgcccagggtgtttaaagaacgtggact

SEQ ID NO: 462 - DNA, 52 bp, Artificial sequence
  A DNA staple used to construct the cube
aggaatggttcgaattcgagcgagatagggttgaacccatcgccaccctcag

SEQ ID NO: 463 - DNA, 31 bp, Artificial sequence
  A DNA staple used to construct the cube
ccgtctatcaactcgtcccgactttcttgcg

SEQ ID NO: 464 - DNA, 37 bp, Artificial sequence
  A DNA staple used to construct the cube
ctctggtttacacagaatatgttaaagaacgtggact

SEQ ID NO: 465 - DNA, 52 bp, Artificial sequence
  A DNA staple used to construct the cube
tgcgggtccgagatcgtcgctgagatagggttgaacccatcgccaccctcag

SEQ ID NO: 466 - DNA, 31 bp, Artificial sequence
  A DNA staple used to construct the cube
ccgtctatcaagtttctctgaccgagggata

SEQ ID NO: 467 - DNA, 37 bp, Artificial sequence
  A DNA staple used to construct the cube
tgagcgtaatagaaccatatcttaaagaacgtggact

SEQ ID NO: 468 - DNA, 52 bp, Artificial sequence
  A DNA staple used to construct the cube
gcttatgtttaccgtagacgcgagatagggttgaacccatcgccaccctcag

SEQ ID NO: 469 - DNA, 31 bp, Artificial sequence
  A DNA staple used to construct the cube
ccgtctatcaggagaaacgtctcccatagta

SEQ ID NO: 470 - DNA, 37 bp, Artificial sequence
  A DNA staple used to construct the cube
ctctatgaagccggccctatattaaagaacgtggact

SEQ ID NO: 471 - DNA, 52 bp, Artificial sequence
  A DNA staple used to construct the cube
aaagagtaactttcagtcagggagatagggttgaacccatcgccaccctcag

SEQ ID NO: 472 - DNA, 31 bp, Artificial sequence
  A DNA staple used to construct the cube
ccgtctatcagtgagcaacactacatcctca

SEQ ID NO: 473 - DNA, 37 bp, Artificial sequence
  A DNA staple used to construct the cube
cctgctataggaatcacggagttaaagaacgtggact

SEQ ID NO: 474 - DNA, 44 bp, Artificial sequence; identical to SEQ ID NO: 232
  A sequence used to generate a 200nm bar

SEQ ID NO: 475 - DNA, 22 bp, Artificial sequence
  A sequence used to generate a 200nm bar
cgctgacatcggttctaatgcc

SEQ ID NO: 476 - DNA, 44 bp, Artificial sequence; identical to SEQ ID NO: 234
  A sequence used to generate a 200nm bar

SEQ ID NO: 477 - protein, 12 aa, Homo sapiens
IGEVWSKMEKGG

SEQ ID NO: 478 - protein, 10 aa, Homo sapiens
GMSTRSKTKP

SEQ ID NO: 479 - protein, 10 aa, Homo sapiens
ESDKPETRKR

SEQ ID NO: 480 - protein, 15 aa, Homo sapiens
MTMGHKYRMKGEPLE

SEQ ID NO: 481 - protein, 9 aa, Homo sapiens
KMKKYSEYW

SEQ ID NO: 482 - protein, 9 aa, Homo sapiens
HEGPHESTE

SEQ ID NO: 483 - protein, 7 aa, Influenza virus
SSGEMRW

SEQ ID NO: 484 - protein, 7 aa, Influenza virus
STEEEEW

SEQ ID NO: 485 - protein, 7 aa, Influenza virus
SFSEMTW

(19) United States
(12) Patent Application Publication
(10) Pub. No.: US 2014/0341975 A1
(43) Pub. Date: Nov. 20, 2014

(54) PATHOGEN AND SUBSTANCE TRAPS

(71) Applicant: Erez Aharon Livneh, Pardesiya (IL)

(72) Inventor: Erez Aharon Livneh, Pardesiya (IL)

(73) Assignee: VICOY NANOMEDICINES LTD, Pardesiya (IL)

(21) Appl. No.: 14/240,745

(22) PCT Filed: Aug. 23, 2012
(86) PCT No.: PCT/IL2012/050326

§ 371 Date: Feb. 25, 2014

Related U.S. Application Data
(60) Provisional application No. 61/527,623, filed on Aug. 26, 2011.

(51) Int. Cl. A61K 47/48; C07H 21/04; A61K 45/06; A61K 9/127

(52) U.S. Cl. CPC A61K 47/48176; A61K 9/1271; C07H 21/04; A61K 45/06
USPC 424/450; 424/78.18; 536/23.1; 525/333.3; 525/54.1

(57) ABSTRACT

A composition-of-matter is provided as well as pharmaceutical compositions and methods of using same. The composition of matter includes least one active moiety surrounded by a scaffold configured for enabling selective influx of an agent capable of interacting with the at least one active moiety.

### FIELD AND BACKGROUND OF THE INVENTION

The present invention relates to a complex for sequestering and binding or processing substances or pathogens.

Efficient management of viral infections remains one of the largest challenges to present day biomedicine. The contemporary Swine Flu and HIV pandemics illustrate that modern science has yet to devise effective agents capable of containing and treating viral outbreaks.

Despite the vast number of viral illnesses, there are but a few specific anti-viral therapies available. To date, vaccines are regarded as the most effective form of viral treatment, however, vaccines can be ineffective or may carry the risk of adverse reactions which can be lethal. Other than vaccines, there is but a small set of pharmaceuticals that are designed to attenuate viral infections typically through inhibition of viral or host enzymes. These small molecule inhibitors can carry a risk of toxic side effects and due to rapid mutations in viral populations, can lead to formation of resistant strains.

Thus, antiviral therapies typically involve either prophylactic activation of the immune system prior to an infection or targeting of virally infected cells via small molecule inhibitors. Little attention has been given to the inactivation of viruses prior to their infection of host cells.

One such approach involves inactivation of viruses through host cell mimicry, by using liposomes, cells or protocells carrying membrane-bound virus receptors. Such an approach relies on virus-host interactions which are evolutionarily conserved even in the face of rapid viral adaptation.

Receptor-bearing liposomes (proteo-liposomes) are potential virus targets, yet are evolutionary traps, since viruses cannot reproduce within them and therefore are inactivated. The concept of liposome traps was initially described in DE3711724, where a liposome trap was suggested as an HIV inactivator. Similar suggestions were made in publications ES2088752 and WO 1996/022763. U.S. Pat. No. 5,718,915 describes the addition of a catalytic enzyme to a liposome membrane in order to induce damage to bound viruses. Other similar antiviral systems employing proteo-liposomes as drug delivery systems have also been described Bronshtein et al. (2011, Journal of Controlled Release, Vol. 51, Issue 2, p.139-148) as well as U.S. Pat. No. 5,773,027, US 2008/0138351, U.S. Pat. No. 6,544,958, Tuthill et al. (2006 doi:10.1128/JVI.80.1.172-180.2006) and Zhukovsky et al. [2010 PLoSOne, 5(10) e13249].

Use of non-host cells presenting viral receptors (typically RBCs) as viral traps, was described in EP 0 298 280. In such an approach, the nucleus-lacking RBCs serve as a trap within which the infectious viral particle can not multiply. Similar approaches were described in publications U.S. Pat. No. 5,677,176, U.S. Pat. No. 7,462,485 and in the “OpenWetWare 20.380 HIV Project”.

Artificial cell-like particles termed protocells can also be used to present virus specific receptors [Porotto et al. (2011) PLoSOne, 6(3): e16874.

Other extra-cellular virus-inactivation approaches using antiviral dendrimers [Reuter et al. (1999) Bioconjugate Chem. 10(2): 271-8], and protein nanotubes [Komatsu et al. (2011) J Amer Chem Soc. 133(10): 3246-8] are also under investigation.

Although these approaches can be effective against specific viruses, none can provide a solution for a wide spectrum of viral diseases. in addition, these approaches can be limited by undesired interaction with in vivo cells/molecules that can lead to adverse toxic effects, poor pharmacokinetics and instability of the antiviral agent, and rapid clearance of the antiviral agent from the body by the immune system and other tissues.

Therefore, there remains a need for an approach that can be used to treat a wide range of pathogen infections without the aforementioned limitations of prior art approaches.

### SUMMARY OF THE INVENTION

According to one aspect of the present invention there is provided a composition-of-matter comprising at least one active moiety surrounded by a scaffold configured for enabling selective influx of an agent capable of interacting with the at least one active moiety.

According to further features in preferred embodiments of the invention described below, the scaffold includes a nucleic acid, a polymer, and/or a silicon structure.

According to still further features in the described preferred embodiments the nucleic acid structure is a DNA origami structure.

According to still further features in the described preferred embodiments the structure is a Micro Electro Mechanical System (MEMS) structure.

According to still further features in the described preferred embodiments the agent is a substance, a microorganism or a cell.

According to still further features in the described preferred embodiments the microorganism is selected from the group consisting of a virus, a bacteria, a fungus, a parasite, an archae, an algea and a protist.

According to still further features in the described preferred embodiments the substance is selected from the group consisting of a peptide, a polypeptide, a prion, a lipid, a lipid complex, a nucleic acid, a carbohydrate, an allergen, a toxin, a hormone, an antibody, a drug, a small molecule, a pollutant and a mineral.

According to still further features in the described preferred embodiments the scaffold is configure to allow selective influx of an agent having a diameter of 0.01-50 μm.

According to still further features in the described preferred embodiments the scaffold is configured to allow selective influx of an agent having a diameter of 0.01-0.8 μm.

According to still further features in the described preferred embodiments the active moiety is capable of binding the agent.

According to still further features in the described preferred embodiments the active moiety is selected from the group consisting of an antibody, an aptamer, a receptor, a chelator, a ligand, a liposome, nanotube, a dendrimer, a protocell, a cell, a peptide, a protein, an enzyme, a chemical, a detergent, a toxin, a drug and a prodrug.

According to still further features in the described preferred embodiments the active moiety is capable of cleaving or deforming the agent.

According to still further features in the described preferred embodiments the active moiety is selected from the group consisting of an enzyme, a ribozyme, a chemical, an acid, a base and a detergent.

According to still further features in the described preferred embodiments the active moiety is capable of binding a microorganism by a moiety not endogenous to the microorganism.

According to still further features in the described preferred embodiments the at least one active moiety is attached to the scaffold.

According to still further features in the described preferred embodiments the at least one active moiety is attached to the scaffold via a linker.

According to still further features in the described preferred embodiments the at least one immune-modulating moiety is attached to the scaffold.

According to still further features in the described preferred embodiments the scaffold is substantially non-immunogenic in a vertebrate.

According to still further features in the described preferred embodiments the scaffold is a non-lipid scaffold.

According to still further features in the described preferred embodiments the scaffold includes PEG or a PEG derivative, or hyaluronic acid.

According to still further features in the described preferred embodiments the scaffold forms a particle having an internal lumen.

According to still further features in the described preferred embodiments the at least one active moiety is disposed in the lumen.

According to still further features in the described preferred embodiments the at least one active moiety is attached to the scaffold in the lumen.

According to still further features in the described preferred embodiments the at least one active moiety is attached to a carrier in the lumen which is bound by its size to the scaffold.

According to still further features in the described preferred embodiments the at least one active moiety is capable of releasing a molecule following interaction with the agent.

According to still further features in the described preferred embodiments the molecule is selected from the group consisting of a marker, a toxin, a hormone, a drug, a nucleic acid, a protein and an adjuvant.

According to still further features in the described preferred embodiments the scaffold further comprises a targeting moiety.

According to still further features in the described preferred embodiments the targeting moiety is selected from the group consisting of a receptor, an antibody, an aptamer, a tissue-specific moiety, a microorganism-specific moiety, a ligand, and a magnet.

According to another aspect of the present invention there is provided a pharmaceutical composition comprising the composition-of-matter and pharmaceutically acceptable carrier.

According to still further features in the described preferred embodiments the pharmaceutically acceptable carrier is selected suitable for oral, mucosal, topical or systemic delivery.

According to another aspect of the present invention there is provided a method of isolating an agent from a fluid comprising exposing the fluid to a composition-of-matter including at least one active moiety surrounded by a scaffold configured for enabling selective influx of the agent capable of interacting with the at least one active moiety, thereby isolating the agent from the fluid.

According to still further features in the described preferred embodiments the fluid is a biological fluid.

According to still further features in the described preferred embodiments exposing the biological fluid to a composition-of-matter is effected by administering the composition-of-matter to a subject.

According to still further features in the described preferred embodiments the fluid is an aqueous fluid.

According to still further features in the described preferred embodiments the composition-of-matter is part of a filter positioned within or on a container.

According to still another aspect of the present invention there is provided an isolated polynucleotide comprising the sequence set forth in SEQ ID NO: 209-476.

According to still another aspect of the present invention there is provided an microparticle including the isolated polynucleotide of the present invention.

According to still another aspect of the present invention there is provided an isolated polypeptide comprising the sequence set forth in SEQ ID NO: 477-485.

According to still another aspect of the present invention there is provided a microparticle including the isolated polypeptide of the present invention.

The present invention successfully addresses the short comings of the presently known configurations by providing a composition-of-matter and methods of using same for preventing or treating pathogen infections as well as purifying substances from biological and non-biological liquids.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a-b schematically illustrates one embodiment of a complex for capturing entities constructed in accordance with the teachings of the present invention.

FIG. 1c schematically illustrates another embodiment of a complex for capturing entities constructed in accordance with the teachings of the present invention.

FIG. 2 schematically illustrates another embodiment of a complex for capturing entities constructed in accordance with the teachings of the present invention.

FIG. 3a-c schematically illustrates virus capture using the complex of FIG. 1c. A red blood cell is illustrated on the right to demonstrate size.

FIG. 4a is a light microscope image of non-porous polystyrene microspheres.

FIGS. 4b-c are scanning electron microscope (SEM) images of the non-porous polystyrene microspheres.

FIGS. 5a-b are scanning electron microscope (SEM) images of a mixture of non-porous and porous polystyrene microspheres.

FIGS. 5c-f are scanning electron microscope (SEM) images of porous polystyrene microspheres.

FIGS. 6a-b are flow cytometry readings of the porous polystyrene microspheres. The FL2-A readings (FIG. 6a) correspond to emissions at the yellow-orange spectra, while the FL4-A readings (FIG. 6b) correspond to emission at the far red spectra. FIGS. 7a-b illustrate an uninfected SF9 cell culture. Magnification ×100—FIG. 7a; ×400 FIG. 7b.

FIGS. 8a-b illustrate an SF9 cell culture incubated with Baculovirus. Magnification ×100—FIG. 8a; ×400 FIG. 8b.

FIGS. 9a-b illustrate a treated SF9 cell culture, incubated with Baculovirus and antiviral Polystyrene microspheres. Magnification ×100—FIG. 9a; ×400 FIG. 9b. A sample of Polystyrene microspheres is indicated by arrows (FIG. 9b).

FIG. 9c is a graph illustrating in vitro rescue of baculovirus associated infection in Sf9 cells by the virus-traps of the present invention as measured by GFP relative fluorescence.

FIG. 9d is a graph illustrating in vitro rescue of baculovirus associated infection in Sf9 cells by the virus-traps of the present invention as measured by GFP relative fluorescence.

FIG. 10a illustrates injection of a Blaberus craniifer cockroach by the virus-traps of the present invention.

FIG. 10b is a graph illustrating in vivo rescue of baculovirus associated infection in Blaberus craniifer cockroaches by the virus-traps of the present invention as measured by GFP relative fluorescence.

FIGS. 11a-b are scanning electron microscope (SEM) images illustrating adhesion of Baculovirus virions to Triton-coated porous Polystyrene microspheres.

FIG. 12a is a transmitting electron microscope (TEM) image of a DNA Bucky ball generated according to the teachings of the present invention; the spherical shape has an estimated size of a Bucky ball (500-800 nm).

FIG. 12b schematically illustrates the Bucky ball shape formed by the self assembling connector DNA of the present invention.

FIG. 13 illustrates DNA-Origami constructed corners of a DNA cube used as a scaffold in a virus trap of the present invention.

FIG. 14 illustrates the predicted interaction of an in silico generated peptide to virion presented host-specific protein Human CD81.

### DESCRIPTION OF THE PREFERRED EMBODIMENTS

The present invention is of a trap which can be used to treat or prevent pathogen infections as well as to detoxify or clean biological fluids, water supplies and the like. Specifically, the present invention can be used to trap and inactivate viral particles thus preventing viral replication and infection in a host or sample.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Pathogen infection and in particular viral infections that lead to chronic diseases present multiple unmet challenges to viral immunologists.

Although there are several antiviral agents currently in use, viruses that cause chronic disease lack effective prophylactic and treatment agents. Numerous anti-viral agents are in development in efforts to provide suitable treatment to infected individuals. Such agents include inhibitors of viral enzymes, inhibitors of host cell virus-binding, antisense and RNA interference agents, immunomodulation agents and virus maturation inhibitors. Such antiviral agents can be limited by inefficient delivery to target cells and tissues, short therapeutic window due to rapid clearance from circulation, toxicity and immunogenicity and adaptation and resistance of virus to agents.

Approaches for purifying agents from fluids such as biological fluids are well known in the art. Such approaches can be used to selectively purify or trap viruses in biological fluids by using size exclusion or affinity binding techniques.

Agents capable of affinity binding viruses include Proteo-Liposomes or protocells displaying receptors that bind microorganisms, dendrimers that bind microorganisms and impurities, antibodies and antibody-complexes that bind to microorganisms and impurities and protein nanotubes that bind microorganisms and impurities.

Although such approaches can potentially be used to purify undesirable agents such as viruses from a subject's blood, they suffer from several drawbacks including rapid clearance from the bloodstream by phagocytes, potential toxic/immunogenic/pathologic side effects caused by the foreign material, failure to inactivate/sequester the agent captured, narrow range of capturable agents (e.g. viruses that infect host cells by means of an endocytosis mechanism will not be inactivated by prior art approaches) and a limited number of agents that can be bound by a single trapping molecule/complex.

While reducing the present invention to practice, the present inventor realized that robust and efficient purification of an agent from a fluid such as blood using, for example, an in-vivo approach requires a two step cooperative process that first isolates/sequesters the agent from the fluid and then traps and optionally processes it.

To enable such functionality, the present inventor designed a purification composition (also referred to herein as “complex”) which is specifically configured for first isolating and sequestering the agent from the fluid and then trapping and inactivating it (e.g. by binding or processing it).

Such a two step approach is particularly advantageous for in-vivo prevention or treatment of a pathogen infection. In such cases, since the complex is designed to first sequester (via, for example, size exclusion influx) and then process the pathogen within an internal and thus immuno-protected volume of the complex, the moieties responsible for binding/inactivating the pathogen (which are positioned within such an internal volume) are not presented to the host cells and thus cannot elicit an immunological or toxic reaction.

The present invention overcomes the limitations of present day anti-viral treatments which do not adequately address viral adaptive resistance. Viruses are highly adaptive and polymorphic and are capable of undergoing substantial genetic changes that give rise to alteration of their protein domains, and specifically their enzymatic active sites, leading to a resistance to antiviral drugs targeting these proteins. Moreover, viruses also rapidly alter their exterior virion landscape (e.g. by assimilating host cell proteins) to elude the adaptive immune system and specifically antibodies.

For example, viruses treated by the present viral trap which includes host cell receptor moieties (e.g. a raft-like liposome bearing the Human receptors CD4, CCR5 and CXCR4, for the inactivation of the HIV virus) are unlikely to develop adaptive resistance, while viruses which are non-reactive to such receptors are selectively pressured towards mutations that will inhibit their ability to react and infect host cells. Viruses treated with viral traps which include moieties which bind proteins non-endogenous to the virus (e.g. host cell proteins) are unaffected by the rapid and adaptive polymorphism of a virus. Active moieties that specifically bind such host cell moieties, (e.g Tetraspanins, Annexines) are able to bind a wide range of viruses.

By sequestering these virus-binding moieties within non-immuno reactive scaffolds, the present invention limits undesired interaction with host cells and separates the bound virus particles from host cells thereby rendering them non-infective.

Thus, according to one aspect of the present invention there is provided a composition-of-matter which includes at least one active moiety surrounded by a scaffold configured for enabling selective influx of an agent capable of interacting with the active moiety or moieties.

The scaffold of the composition-of-matter can be constructed from any substance capable of forming a particle-like configuration (i.e. having a 3-D shape) having an internal volume and surface pores fluidly communicating therewith. As is further described hereinunder, such a particle can be constructed from synthetic or biological polymers, lipids, zeolites, inorganic material (e.g. silicon), metals using well known approaches such as Polymer Chemistry, DNA Nanotechnology (e.g. DNA Origami) and Micro Electro Mechanical System (MEMS). The Examples section which follows describes several types of particles and their construction.

The active moiety can be any moiety which is capable of interacting and inactivating a broad range of substances or pathogens (e.g. a detergent in the case of proteins or membranes) or a specific pathogen or substance (e.g. antibody or antibody fragment).

The scaffold can include any number of pores of any size depending on the substance or pathogen targeted for sequestering. Preferably, the scaffold is provided with at least three pores. The pores can be designed to passively or actively sequester the substance or pathogen. For example, a scaffold having a pore diameter of 17 nm will enable influx of a virus such as the porcine circovirus, while keeping out living eukaryotic cells and bacteria. A scaffold with openings of 400 nm will enable influx of most viruses, while keeping out eukaryotic cells. A scaffold with openings of 800 nm will enable influx of all viruses and most mycoplasma bacteria cells, while keeping out human cells. A scaffold with openings of 4000 nm will enable influx of most bacteria while keeping out human cells outside of the complex. Also, a scaffold having a pore diameter of 10 nm will enable influx of prions such as the PrP protein, while keeping out most proteins and all viruses, living eukaryotic cells and bacteria.

Thus, in one particular embodiment, pores having a diameter of about 10 to 800 nm would be suitable for trapping viruses, pores having a diameter of about between 0.2-5 μm would be suitable for trapping bacteria, and pores having a diameter of about between 1-20 μm would be suitable for trapping fungi.

Thus, the scaffold of the complex of the invention may have pores with a minimum diameter of 1, 1.5, 2, 2.5, 3, 3.5, 4, 4.5, 5, 5.5, 6, 6.5, 7, 7.5, 8, 8.5, 9, 9.5, 10 10.5, or 11 nm; and pores with a maximum diameter of 5, 6, 7, 8, 9, 10, 11, 12, 13, 14, 15, 16, 17, 18, 19 or 20 μm. Usually, the scaffold pores have a diameter of 50 nm, 100 nm, 200 nm, 300 nm, 400 nm, 500 nm, 600 nm, 700 nm, 800 nm, 900 nm, 1 μm, 1.5 μm, 2 μm, 2.5 μm, 3 μm, 3.5 μm, 4 μm, 4.5 μm, 5 μm, 10 μm, 20 μm, 30 μm, 40 or 50 μm.

Thus, the scaffold functions in selective capturing of entities, through size-selection, and thus functions as a size exclusion ‘filter’. Only those entities small enough to be able to pass through the scaffold pores are captured, and internalized.

Once sequestered within an internal lumen of the scaffold, the substance or pathogen is presented to the active moiety for processing. Thus the scaffold serves two purposes, sequestration and separation of substances or pathogens thus shielding the substance or pathogen from the body and vise versa and shielding the active moiety from host cells and thus preventing toxic effects and particularly from cells of the immune system thus preventing an immune response against the moiety.

Immuno-isolating the active moiety increases its half life and thus the therapeutic effectiveness of the complex of the present invention in vivo, while also enabling use of otherwise immunogenic and possibly toxic active moieties in vivo.

The active moiety (e.g. receptor, enzyme) can be an immunogenic molecule and as such, display of such a molecule on the surface of a particle (e.g. liposome) or carrier, as is the case with some prior art virus traps, can increase the likelihood of an immune reaction to the trap and partial or full inactivation thereof.

The two step isolation and trapping composition of the present invention traverses this limitation of the prior art by sequestering the active moiety within a hollow particle. This shields the active moiety from the immune system of the subject and increases the potential half life of the composition-of-matter in the body. By separating immuno-isolation from trapping and optionally inactivation, the present complex shields potentially immuno-reactive molecules from the subject's immune system and enhances the circulation time of the composition of the present invention.

Furthermore, prior art virus traps such as antibodies and dendrimers, or receptor-bearing proteo-liposomes, can still be infectious/immunoreactive to host cells since the bound virus is still presented to the host cells. The present isolation approach is two-directional, it not only masks the virus and virus-binding moiety from the immune system but it also masks the uninfected host cells from the viruses contained by the virus traps.

The complex of the present invention can be used in-vivo or in-vitro to detoxify or prevent infection in any sample of fluid including a biological fluid such as blood, urine, semen, saliva, mucous, lymph and the like, a non-biological fluid such as drinking water, beverages, sewage, and the like.

One presently preferred use for the present invention is in preventing or treating infection of a pathogen such as a virus in a biological fluid such as blood. In such cases, the present complex functions as a therapeutic agent.

Thus, the present invention provides a novel approach for eliminating pathogens and substances from a liquid medium such as blood without exposing the medium to potentially harmful or immunological chemicals.

As is mentioned hereinabove, the complex of the present invention is three dimensional in shape and includes an internal lumen having one or more active moieties positioned therein.

One specific configuration of a complex that provides such ‘architecture’ includes 4 micron polystyrene hollow microspheres (the scaffold) having pores of 800 nm in diameter and an exterior PEG coating (immuno-isolation). These microspheres encapsulate 1 micron polystyrene microspheres coated with a triton detergent (the moiety). An alternative configuration can include a 1 micron DNA Bucky ball (scaffold) externally coated with mPEG (immuno-isolation) and internally coated with surfactant-Protein-D (moiety).

The scaffold of the complex of the present invention can be fabricated from a nucleic acid nanostructure, a polymer, or a silicon wafer.

Nucleic acid (DNA/RNA) nanostructures are structures whose building blocks are nucleic acids, nucleotides or nucleosides. Nucleic acid nanotechnology makes use of the fact that, due to the specificity of Watson-Crick base pairing, only portions of the strands which are complementary to each other will bind to each other to form duplex. Construction of nucleic acid nanostructures has been described in several publications, including WO 2008/039254, US 2010/0216978, WO 2010/0148085, U.S. Pat. No. 5,468,851, U.S. Pat. No. 7,842,793, Dietz et al. (2009) [Dietz et al. (2009) Science, Vol. 325, pp. 725-730], Douglas et al. (2009) [Douglas et al. (2009) Nature, Vol. 459, pp. 414], amongst others. Examples 6 and 9-11 of the Examples section which follows describe sequences and approaches for generating DNA scaffolds suitable for use with the present invention.

Essentially, natural or artificial sequences of DNA or RNA can be programmed to generate a three-dimensional (3D) structure. Usually, DNA-based nanostructures make use of a single strand of DNA which is induced into a 3D conformation by the binding of complementary, shorter DNA strands. In contrast, RNA folds into 3D by forming tertiary RNA motifs, based on RNA-RNA interactions within the same molecule. Nanostructures based on folded single-stranded DNA are also feasible. RNA duplexes are an alterative for generating RNA 3D structures.

Hence, in one particular embodiment of the scaffold of the invention, the nucleic acid nanostructure is DNA origami. DNA origami is a method of generating DNA artificially folded at nano scale, creating an arbitrary three dimensional shape that may be used as a scaffold for trapping inside, or capturing, an entity. Methods of producing DNA nanostructures of the origami type have been described, for example, in U.S. Pat. No. 7,842,793. DNA origami involves the folding of a long single strand of viral DNA (for example) aided by multiple smaller “staple” strands. These shorter strands bind the longer strand in various places, resulting in the formation of a 3D structure.

The nucleic acid nanostructure of the invention may thus be a structure of joined tiles of DNA origami and/or it may have an inducible shape. Inducible nucleic acid nanostructures have been described, for example, by Andersen et al. (2009) [Andersen et al. (2009) Nature, vol. 459, pp. 73-77], Dietz et al. (2009) [Dietz et al. (2009) Science, Vol. 325, pp. 725-730], Voigt et al. (2010) [Voigt et al. (2010) Nature Nanotechnology, vol. 5, pp. 200-203], and Han et al. (2011) [Han et al. (2011) Science, Vol. 332, pp. 342-346]. A software package for designing nucleic acid nanostructures is available at www.cdna.dk/origami.

As referred to herein, the nucleic acid used in the nucleic acid nanostructure of the complex of the invention refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. Thus, the terms nucleoside, nucleotide, deoxynucleoside and deoxynucleotide generally include analogs such as those described herein.

Typically a nucleic acid will comprise phosphodiester bonds, however, nucleic acids may comprise a modified backbone comprising, for example, phosphoramide, phosphorothioate, phosphorodithioate,

O-methylphophoroamidite linkages, and peptide nucleic acid backbones and linkages. Other analog nucleic acids include those with positive backbones; non-ionic backbones and non-ribose backbones. Nucleic acids containing one or more carbocyclic sugars are also included within the definition of nucleic acids. As will be appreciated by those in the art, all of these nucleic acid analogs may find use as helper strands or as part of a polynucleotide used to generate the nanostructure. In addition, mixtures of naturally occurring nucleic acids and analogs can be made. PNA (Peptide nucleic acids) includes peptide nucleic acid analogs, which have increased stability.

Thus, nucleic acid of various forms and conformations may be used for generating the nanostructure scaffold, including right-handed DNA, right-handed RNA, PNA, locked nucleic acid (LNA), threose nucleic acid (TNA), glycol nucleic acid (GNA), bridged nucleic acid (BNA), phosphorodiamidate morpholino oligo (PMO), as well as nucleotide analogues, such as non-Watson-Crick nucleotides dX, dK, ddX, ddK, dP, dZ, ddP, ddZ.

In some embodiments, a nanostructure of the invention including a polynucleotide may comprise one or more distinct polymeric nucleic acid structures (e.g., at least 20, at least 50, at least 100, or at least 1000 or more distinct nucleic acid molecules). The nucleic acids may be single stranded or double stranded, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, and the like. Such nucleic acids comprise nucleotides and nucleoside and nucleotide analogs, and modified nucleosides such as amino modified nucleosides.

The DNA nanostructure of the invention may use numerous short single strands of nucleic acids (helper strands) (e.g., DNA) to direct the folding of a long, single strand of polynucleotide (which is called, in DNA nanostructure nomenclature, the scaffold strand) into desired shapes that are usually between 100-5000 nm in diameter. Thus, the nucleic acid scaffolds of the complex of the invention may be on the order of about 100 nm to 5000 nm, but larger scaffolds of 10, 15 or 20 μm may also be used, depending on the context.

In another embodiment, the scaffold of the complex of the invention may be a polymeric structure, wherein the building blocks are polymers such as polyvinylalcohol (PVA), Polylactide (PLA), Poly L-D-Lactide-co-Glycolide (PLGA), Dimethylaminoethyl methacrylate methyl methacrylate copolymer, PAN [Xiang et al., Hazard Mater. 2010 Jan. 15; 173(1-3):243-8], or PMMA (Yuan et al., Langmuir. 2009 Mar. 3; 25(5):2729-35; Zhang et al., Colloid Interface Sci. 2009 Aug. 1; 336(1):235-43; Lin et al. Langmuir. 2008 Dec. 2; 24(23):13736-41], Poly(ortho esters) (POE) [Heller et al. (2000) J. Mater. Sci Mater. Med. 11(6):345-55], Polyphosphazenes [Allcock (1994) Biomaterials, 15(8):563-9], Polyanhydrides (Shieh et al. (1994) J. Biomed. Mater REs. 28(12):1465-75, Polyphosphoesters [Richards et al. (1991) J. Biomed. Mater. Res. 25(9):1151-1167] Polyglycolide (PGA), polytrimethylene carbonate (PTMC), poly(L-lactic acid) and poly(glycolic acid) (PLLA/PGA), PGA, PLLA-PGS, 1,3-Propanediol (PDO), polyethylene, polyketones (PEEK), Alginates, Alginate with Poly L-Lysine (alginate/PLL), Sodium Alginate, Agarose, Hyaluronic acid, hydrogels, such as Hydroxyethylmethacrylate (HEMA), Hydroxyethylmethacrylate methyl methacrylate (HEMA-MMA), Methacrylic acid, Methyl methacrylate, chitosan, collagen, cellulose polymers, amongst others. These polymers may function as semi-permeable membranes, and are known as biosafe membranes.

In a further embodiment, the scaffold of the complex of the invention is a silicon microstructure, for example a silicon wafer forming a biocapsule. A method for the production of silicon wafers has been described, e.g. by Desai et al. [Desai et al. (1998) Biotechnology and Bioengineering, vol. 57, no. 1, pp. 118-120].

In another further embodiment, the scaffold of the invention may further comprise targeting moieties for targeting the scaffold to specific tissues in vivo. Such targeting moieties can be, for example, tissue-specific ligands or receptors, or other tissue- or cell-specific molecules, which may for example bind to the extracellular matrix of the target tissue.

In another further embodiment, the scaffold of the invention may further comprise molecules (toxins for example) which enable the same to kill cells, such as phagocytes, in situations when the complex is engulfed or phagocytosed by the same.

Thus, the scaffold of the invention may be provided with immune-modulator, or toxic or cytotoxic moieties which facilitate or induce cell death, such as alendronate, clodronate, AppCCl2p (clodronate metabolite), DMDP (methyl-5-deazapteridine), the sequence or product of a suicide gene, etc.

The scaffold of the present invention can fabricated from non-immunogenic materials such as PLGA [Lin et al. Biomaterials. 2012 July; 33(20):5156-65]; PVA [Efthimiadou et al., Int J Pharm. 2012 May 30; 428(1-2):134-42], chitosan [Mu et al., Mol Pharm. 2012 Jan. 1; 9(1):91-101; Lu et al., Biointerfaces. 2011 Apr. 1; 83(2):254-9], cellulose [Mctaxa et al., J. Colloid Interface Sci. 2012 May 18], collagen [Hclary et al., Acta Biomater. 2012 Jun. 15. [Epub ahead of print]. Alternatively, the scaffold can be coated with a non-immunogenic material. For example, the scaffold can be coated with polyethylene glycol (PEG) or derivatives thereof (Ref: Macromol Biosci. 2004 May 17; 4(5):512-9.

The scaffold can be provided with targeting moieties, in order to target the complex of the invention to specific tissues. Such moieties can be used to target the complex of the present invention to tissues which are infected

The targeting moiety can be a tissue-specific moiety, a virus-specific receptor, an antibody, a ligand, a carbohydrate, a protein, a peptide, a lipid, or a magnetic moiety.

According to one preferred embodiment, the scaffold is provided with liver-specific ligands on its exterior, directing the complex of the invention to the liver. Similarly, other tissues may be the target, such as the pancreas, heart, spleen, kidneys, lymph nodes, etc.

The active moiety can be any molecule or structure capable of specifically or non-specifically binding/processing the substance or pathogen.

The active moiety can be a liposome, a nanotube, an aptamer, a dendrimer, a protein, peptide, a receptor, an enzyme, a ligand, an antibody, a chelator, a detergent, a toxin, a drug or a prodrug.

Liposomes are vesicles made of lipid bilayer, and which may carry on its surface target-specific moieties, or entity binding moieties, such as ligands, receptors, antibodies, carbohydrates, proteins or lipids. Liposomes may present virus-specific receptors on their membranes, such as CD4, CCR5, CXCR4, CCR2, CCR3, Tetraspanin CD81, human scavenger receptor SR-BI, Claudin-1, Occludin, Ephrin-B2, CD46, CAR, av integrin, HAVCR-1, EGFR (epidermal growth factor receptor), SLAM, acetylcholine receptors, neurotrophin receptor, p75 NTR, sialic acid, glycosaminoglycan, heparan sulfate, hyaluronan (hyaluronic acid), collagen, gelatin, polyacrylic acid, chitosan. Alternatively, or in addition, liposomes may carry another moiety in its lumen, such as enzymes, e.g., DNase, RNase, protease, glycosidase, or lipase, amongst others; bases, acids, inhibitors, irreversible binders, etc.

Other active moieties that may function as scavengers are protein nanotubes (for preparation methods see e.g. Qu and Komatsu (2010) ACS Nano, Vol. 4, No. 1, pp. 563-573), dendrimers (for preparation methods see e.g. U.S. Pat. No. 4,289,872; U.S. Pat. No. 4,410,688, U.S. Pat. Nos. 4,507,466, 4,558,120, U.S. Pat. No. 4,568,737, U.S. Pat. No. 4,587,329, U.S. Pat. No. 6,190,650, WO 88/01178, WO 88/01179, and WO 88/01180), aptamers, proteins, enzymes, ligands (e.g. receptors), antibodies, chelators (e.g. EDTA), protocells, toxins, drugs or prodrugs.

As referred to herein, “aptamers” are relatively short nucleic acid (DNA, RNA or a combination of both) sequences that bind with high avidity to a variety of proteins. Aptamers are generally about 25-40 nucleotides in length and have molecular weights in the range of about 18-25 kDa. Aptamers with high specificity and affinity for targets can be obtained by an in vitro evolutionary process termed SELEX (systemic evolution of ligands by exponential enrichment) [see, for example, Zhang et al. (2004) Arch. Immunol. Ther. Exp. 52:307-315 incorporated herein by reference in its entirety].

As referred to herein, “antibodies” relates to naturally derived, or naturally produced antibodies, which may be polyclonal or monoclonal. Alternatively, the antibodies may be synthetically produced by e.g. chemical synthesis, or recombinantly produced through the isolation of the specific mRNA from the respective antibody-producing cell or cell line. The specific mRNA shall then undergo standard molecular biology manipulations (obtaining cDNA, introducing the cDNA into expression vectors, etc.) in order to generate a recombinantly produced antibody. The techniques are well known to the man skilled in the art.

The generation of polyclonal antibodies against proteins is a technique well known to the man skilled in the art, and it is described, inter alia, in Chapter 2 of Current Protocols in Immunology, John E. Coligan et al. (eds.), Wiley and Sons Inc.

The technique of generating monoclonal antibodies is described in many articles and textbooks, such as the above-noted Chapter 2 of Current Protocols in Immunology, Kohler and Milstein [Kohler and Milstein (1975) Nature 256; 495-497], and in U.S. Pat. No. 4,376,110.

The term “antibody” is also meant to include both intact molecules as well as fragments thereof, such as, for example, scFv, Fv, Fab′, Fab, diabody, linear antibody, F(ab′)2 antigen binding fragment of an antibody which are capable of binding antigen [Wahl et al. (1983) J. Nucl. Med. 24, 316-325].

Fab and F(ab′)2 and other fragments of the antibodies useful in the present invention may be tagged with various tags, according to the intended use. These tags may be toxic tags, which would kill the target.

Detergents may act as a cell disruption-like method. A non-limiting list of detergents includes CHAPS, TritonX 100, SDS, Tween, and the like. Detergents act not necessarily as a scavenger, as instead of “trap” or “capture” the entity in a strict sense, they can alternatively or jointly degrade the same. So detergents, similar to enzymes, function by cleavage or degradation of the entity.

Enzymes which may be used for disrupting cell walls include lysozyme, lysostaphin, zymolase, cellulase, mutanolysin, glycanases, proteases, mannose, etc. Other examples include, e.g., hydrolases. As with the detergents above, enzymes do not necessarily function as a scavenger, since they do not “trap” or “capture” the entity in a strict sense, but degrade the same.

Following capturing, the active moiety of the complex of the invention may release a molecule such as, for example, a cytotoxin, a hormone, a drug, an indicator, a nucleic acid, a protein, an adjuvant or a chemical (acid, base). The molecule or chemical can chemically alter the entity (e.g. disrupt membrane or protein structures of a pathogen).

The active moiety can be anchored to a particle or carrier disposed within the scaffold or to the inner side of the scaffold. Such anchoring may be through a covalent bond, affinity interaction such as e.g. biotin-avidinistrepavidin interaction, or by any other anchoring approach. Examples for molecules that may serve as anchors for binding other known specific molecules include, but are not limited to antibodies, ferritin, polyhistidine tag, c-myc tag, histidine-tag, hemagglutinin tag and the like.

According to one preferred embodiment, the anchoring molecule is an antibody, a receptor or a ligand (e.g. Biotin-Avidin).

According to one preferred embodiment, the carrier is a Polystyrene nano sphere.

As is mentioned hereinabove, the present invention enables sequestering and processing of a substance or pathogen.

A substance can be an element, compound or molecule present in a biological or a non-biological liquid. For example, the substance can be a toxin, an impurity or a contaminant present in blood, water, a consumable liquid product such as beer or wine and the like.

The pathogen can be any organism including prions, viruses, bacteria, protists, fungi, archae or other parasites.

When used for in-vivo or ex-vivo treatment of a subject such as a human, the present invention has therapeutic/prophylactic applications, and thus it is useful in protecting or treating a subject in need from a toxin, an undesired molecule/compound or an infection. For example, the present invention can be used to remove a toxin such as Ricin, Staphylococcal enterotoxin B (SEB) or dioxins, an undesired molecule or complex such as LDL (low-density lipoprotein), glucose, auto-reactive antibodies, prions, allergens, tumorogenic factors such as Tumor necrosis factor-alpha (TNF-α) and the like or an infectious agent such as a virus.

According to one preferred embodiment of the present invention, the complex can be used for removing, neutralizing or eliminating microorganisms such as viruses, bacteria, fungi, protist or archea.

Examples of infectious viruses include: Retroviridae (e.g., human immunodeficiency viruses, such as HIV-1, also referred to as HTLV-III, LAV or HTLV-HT/LAV, or HTV-III; and other isolates, such as HTV-LP); Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); to Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridac (e.g., influenza viruses); Bungaviridae (e.g., Hantaan viruses, bunga viruses, phleboviruses and Nairo viruses); Arena viridae (hemorrhagic fever virus); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herperviridae (herpes simplex virus (HSV) 1 and 2, varicella zoster virus, cytomegalovirus (CMV), herpes viruses); Poxyiridae (variola viruses, vaccinia viruses, pox viruses); and Iridoviridae (e.g., African swine fever virus); and unclassified viruses (e.g., the etiological agents of Spongiform encephalopathies, the agent of delta hepatitides (thought to be a defective satellite of hepatitis B virus), the agents of non-A, non-B hepatitis (class 1—internally transmitted; class 2—parenterally transmitted (i.e., Hepatitis C); Norwalk and related viruses, and astroviruses).

Examples of infectious bacteria include: Helicobacter pyloris, Borelia burgdorferi, Legionella pneumophilia, Mycobacteria sps (e.g., M. tuberculosis, M. avium, M. Intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, corynebacterium diphtheriae, corynebacterium sp., Erysipelothrix rhusiopathiae, Clostridium perfringers, Clostridium tetani, Enterobacter erogenes, Chlamydia trachomatis, Klebsiella pneumonia, Pasturella multicoda, Bacteroides sp., Fusobacterium nucleatum, Streptobacillus moniliformis, Treponema pallidium, Treponema pertenue, Haemophilus influenzae, Leptospira, and Actinomyces israelli, amongst others.

Examples of infectious fungi include: Cryptococcus neoformans, Histoplasma capsulatum, Coccidioides immitis, Blastomyces dermatitidis, and Candida albicans, amongst others.

Examples of protist as pathogen are Plasmodium falciparum, which causes malaria, Toxoplasma gondii (Toxoplasmosis), and Leishmania donovani (Leishmaniasis), amongst others.

Examples of peptide moieties that can be used to bind influenza virus particles are provides in the Examples section which follows. Ligands which can be used as moieties for binding microorganisms such as Influenza virus protein Neuraminidase or host proteins that are incorporated to virions or other microorganisms such as Human protein CD81 are described in “Cellular proteins in influenza virus particles” [Shaw et al., PLoS Pathog. 2008 Jun. 6; 4(6):e1000085].

According to one preferred embodiment of the present invention, the complex can be used prophylactically towards a potential infection by a biowarefare agent or a pandemic by removing, neutralizing or eliminating microorganisms if infected, such as viruses, bacteria, fungi, protist or archea.

Examples of viruses used in biowarefare are Pox viruses (e.g. Variola smallpox, Monkeypox), Encephalitis viruses (e.g. Venezuelan equine encephalitis virus, western equine encephalitis virus, eastern equine encephalitis virus), Arenaviridae (e.g. Lassa, Argentine, Bolivian, Brazilian, Venezuelan hemorrhagic fevers), Bunyaviridae (e.g. Rift Valley, Crimean-Congo, Hantaan), and Filoviridae (e.g. Marburg, Ebola), Flaviviridae (e.g. Yellow, Dengue, Kyasanur Forest, Omsk HFs), amongst others.

Similarly, a subject may be in need of eliminating, removing or neutralizing some other entity or substance, not necessary a pathogen, which may be responsible for a pathology, a physiologic disturbance, or an intoxication, such as a nucleic acid, a small molecule, a prion, a protein, a carbohydrate, a lipid, a toxin, a venom, a drug, a poison, an allergen, a metal, or a pollutant; i.e., any substance for which there may be a need or a desire to clear or purge the same from the system. The entities or substances are further defined below.

In this context, a nucleic acid refers to multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As used herein, the term refers to ribonucleotides as well as oligodeoxyribonucleotides. The term shall also include polynucleotides (i.e., a polynucleotide minus the phosphate) and any other organic base containing polymer. Nucleic acid molecules may be from natural sources (e.g., genomic, cDNA, RNA), or may be from recombinant or synthetic sources (e.g., produced by oligonucleotide synthesis).

A small molecule is a low molecular weight organic compound which is by definition not a polymer. The term small molecule, especially within the field of pharmacology, is usually restricted to a molecule that also binds with high affinity to a biopolymer such as protein, nucleic acid, or polysaccharide and in addition alters the activity or function of the biopolymer. The upper molecular weight limit for a small molecule is approximately 800 Daltons which allows for the possibility to rapidly diffuse across cell membranes so that they can reach intracellular sites of action. Very small oligomers are also usually considered small molecules, such as dinucleotides, peptides such as the antioxidant glutathione, and disaccharides such as sucrose.

Prions are infectious agents composed of protein in a misfolded form. Prions are responsible for the transmissible spongiform encephalopathies in a variety of mammals, including bovine spongiform encephalopathy (BSE, also known as “mad cow disease”) in cattle and Creutzfeldt-Jakob disease (CJD) in humans. All known prion diseases affect the structure of the brain or other neural tissue and all are currently untreatable and universally fatal.

As referred to herein, “carbohydrate”, or “saccharide”, may be a monosaccharide, a disaccharide, e.g. glucose, sucrose or lactose, an oligosaccharide, or a polysaccharide, e.g. starch, cellulose, or complex carbohydrates such as glycosaminoglycans.

As referred to herein, the terms “protein” and “proteins” shall be construed to include all polymers of amino acid residues of any length, and thus the term includes polypeptides, as well as conventionally termed proteins which are a subset of polypeptides, and also peptides, which are the shorter, building block polymers which are made from alpha amino acids joined by amide bonds. Proteins generally include any sequence of amino acids for which the primary and secondary structure of the sequence is sufficient to produce higher levels of tertiary and/or quaternary structure. Proteins are distinct from peptides in that peptides lack the capability to form such tertiary and/or quaternary structure. Proteins typically have a molecular weight of at least about 15 kilo Daltons. In the context of this specification, it will be appreciated that the protein may include the L-optical isomer or the D-optical isomer of the amino acids, and may also include synthetic amino acids. Proteins may be further modified by having other chains attached to it, such as carbohydrates (glycoproteins), lipids (lipoproteins), phosphorus (phosphorylated proteins), sulfur, and the like. Proteins that may be trapped or captured by the complex of the invention include antibodies, enzymes, cytokines, hormones, etc. One specific example of a protein that may be desirable to capture with the complex of the invention is amyloid beta. Other molecules such as non-protein cellular communication molecules or neurotransmitters such as cAMP, dopamine, serotonin, epinephrine and the like may also be a target to be reduced or eliminated from a subject's circulation.

As referred to herein, “lipids” include fats, sterols, fat-soluble vitamins (such as vitamins A, D, E and K), monoglycerides, diglycerides, phospholipids, and others. One specific example of a lipid that may be desirable to capture by the complex of the invention is low-density lipoprotein cholesterol (LDL-C).

As referred to herein, “toxin” is a poisonous substance produced by living cells or organisms, and includes hemotoxins, phototoxins, cyanotoxins, necrotoxins, neurotoxins (e.g. brevetoxins), cytotoxins, apitoxins and mycotoxins. Venom is the general term referring to any variety of toxins used by certain types of animals that inject it into their victims by the means of a bite or a sting. A non-exhaustive list of animals that produce venoms includes spiders, scorpions, snakes, fish, octopus, jellyfish, bees, wasps, ants, shrew, mole, amongst others.

As referred to herein, an “allergen” is any substance that can cause an allergy. Typical examples of allergens include pollen, dust mite, pet dander, nuts, perfume, seafood, peanuts, tree nuts, eggs, milk, shellfish, fish, wheat and their derivatives, and soy and their derivatives, as well as sulfites (chemical based, often found in flavors and colors in foods) at 10 ppm and over, fire ants, poison ivy, bee stings, drugs (e.g. penicillin), and latex. Allergens of fungal origin include basidiospore, Pleurotus ostreatus, cladosporium, calvatia cyathiformis, aspergillus and alternaria-penicillin families, fomes pectinatis. The list of allergens is enormous and can also include insect venoms, animal dander dust, fungal spores, etc. Examples of natural, animal and plant allergens include proteins specific to the following genuses: Canine (Canis familiaris), Dermatophagoides (e.g., Dermatophagoides farinae); Felis (Felis domesticus); Ambrosia (Ambrosia artemiisfolia; Lolium (e.g., Lolium perenne or Lolium multiflorum); Cryptomeria (Cryptomeria japonica); Alternaria (Alternaria alternata); Alder; Alnus (Alnus gultinosa); Betula (Betula verrucosa); Quercus (quercus alba); Olea (Olea europa); Artemisia (Artemisia vulgaris); Plantago (e.g., Plantago lanceolata); Parictaria (e.g., Parictaria officinalis or Parictaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis and Juniperus ashei); Thuya (e.g., Thuya orientalis), Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis (e.g., Dactylis glomerata); Festuca (e.g., Festuca elatior); Poa (e.g., Poa pratensis or Poa compressa); Avena (e.g., Avena sativa); Holcus (e.g., Holcus lanatus); Anthoxanthum (e.g., Anthoxanthum odoratum); Arrhenatherum (e.g., Arrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis) and Bromus (e.g., Bromus inermis).

As referred to herein, a “drug” relates to a medical drug or a drug may be understood in the sense of a recreational drug, such as an opiate, alcohol or nicotine.

A medical drug, also referred to in the field as medication or medicament, includes antipyretics, analgesics (painkillers), antibiotics, antiseptics, and the like. Different types of medications are specific to the system to be treated, and a non-exhaustive list of medications includes:: antacids, reflux suppressants, antiflatulents, antidopaminergics, proton pump inhibitors (PPIs), H2-receptor antagonists, cytoprotectants, prostaglandin analogues, laxatives, antispasmodics, antidiarrheals, bile acid sequestrants, opioids, b-locker receptors, calcium channel blockers, diuretics, cardiac glycosides, antiarrhythmics, nitrate, antianginals, vasoconstrictors, vasodilators, peripheral activators, antihypertensive drugs, ACE inhibitors, angiotensin receptor blockers, a blockers, anticoagulants, heparin, antiplatelet drugs, fibrinolytics, antihemophilic factors, haemostatic drugs, atherosclerosis/cholesterol inhibitors, hypo lipidaemic agents, statins, antipsychotics, antidepressants, antiemetics, anticonvulsants/antiepiletics, anxiolytics, barbiturates, movement disorder drugs, stimulants, benzodiazepines, cyclopyrrolones, dopamine antagonists, antihistamines, cholinergics, anticholinergics, cannabinoids, NSAIDS, paracetamol, tricyclic antidepressants, muscle relaxants, androgens, antiandrogens, gonadotropin, corticosteroids, vasopressin analogues.

Thus, the complex of the invention may be used as therapy for overdose caused by medicines or recreational drugs.

As referred to herein, the term “prodrug” refers to a compound that is made active (or more active) upon a trigger. Prodrugs are structurally modified forms of a compound that readily undergo chemical changes under physiological conditions to make the compound available and active. A wide variety of prodrug derivatives are known in the art, such as those that rely on hydrolytic cleavage or oxidative activation of the prodrug. An example, without limitation, of a prodrug would be a compound which is an ester (the “prodrug”), but then is metabolically hydrolyzed to the carboxylic acid, the active entity. Additional examples include peptidyl derivatives of a compound, and etc.

As referred to herein, a “poison” may be a chemical warfare agent (e.g. mustard gas), ricin, cyanide, pesticides, herbicides, amongst others.

Metal poisoning is a serious health concern and thus, means for eliminating unwanted amounts of metals, either in the subject's circulation, or in liquids for human consumption are highly sought after.

Thus, the complex of the invention may also be used for trapping or capturing metals which can cause poisoning when excessive amounts are ingested and/or accumulate in a subject and pollutants. Metals include, e.g., lead, mercury, cadmium, aluminum, bismuth, gold, gallium, lithium, silver, barium salts, polonium, cobalt, manganese, arsenic, chromium, cobalt, copper, iron, nickel, selenium, thallium, and zinc. Pollutants include toxic waste, such as dioxin and its derivatives.

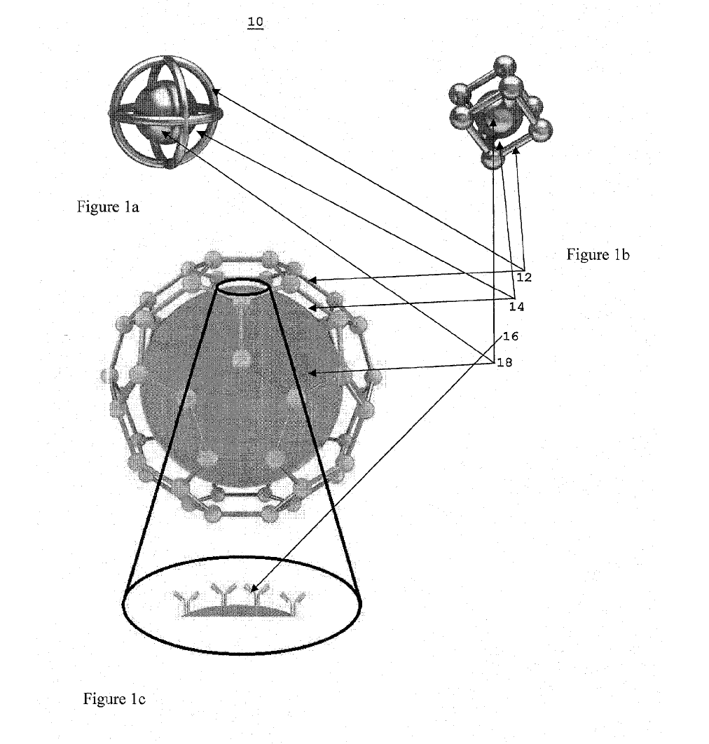

FIGS. 1a-c and 2 illustrate several embodiments of the present complex which is referred to herein as complex 10.

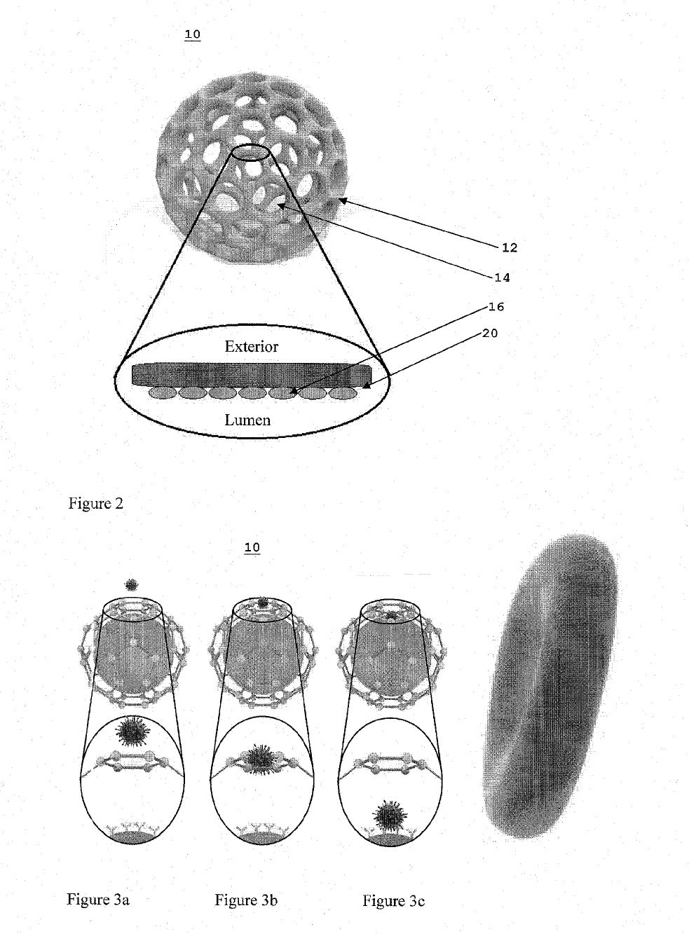

Complex 10 includes a porous scaffold 12 which is arranged as a three dimensional particle having an internal lumen 14. Lumen 14 includes an active moiety 16 which is attached to a carrier 18 (e.g. particle) positioned and bound by its size within lumen 14 (FIG. 1) or to an inner surface 20 of a scaffold 12 (FIG. 2).

Scaffold 12 includes pores 22 having a specific diameter range selected according to the entity targeted for trapping. For example, a scaffold 10 designed for trapping an influenza virus can have pores 22 with a diameter of 100-800 nm.

Scaffold 10 shown in FIG. 1 can be constructed from polystyrene micro/nano-particles as is described in Examples 1-2 of the Examples section which follows. Scaffold 10 shown in FIG. 2 can be constructed using DNA as is described in Examples 9-10 and specifically by the DNA origami technique described in Examples 4 and 11.

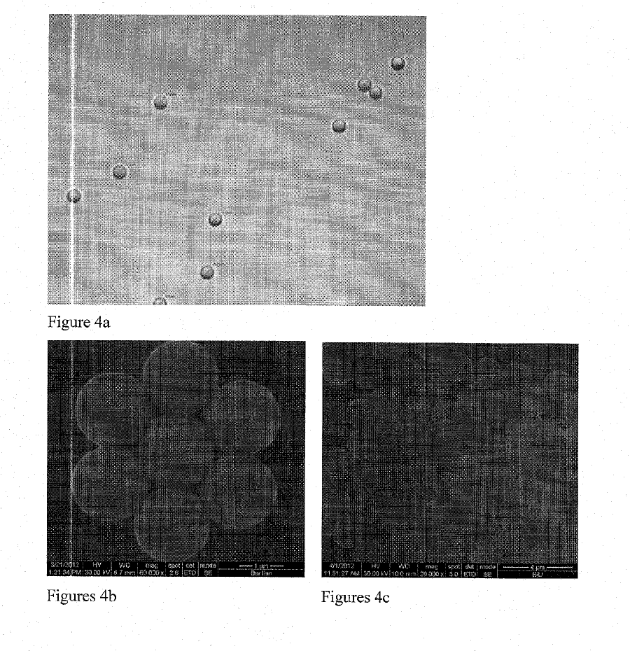

FIG. 3a-c illustrates viral trapping using scaffold 10 of FIG. 1c. FIG. 3a illustrates a virion outside of the scaffold of the complex, FIG. 3b illustrates the selective influx of the virion through the porous scaffold of the complex and FIG. 3c illustrates the interaction of the virion with the active moiety (e.g. antibodies). A red blood cell is illustrated on the right to demonstrate scale.

As is mentioned hereinabove, the complex of the present invention can be useful in in-vitro or in-vivo treatment of a biological fluid (e.g. detoxification or prevention or treatment of an infection) or in treatment of a non-biological fluid (e.g. water purification).

Thus according to another aspect of the present invention there is provided a method of removing a substance or pathogen from a liquid.

One preferred application of the present method is treatment of a subject suffering from or predisposed to a pathogen infection.

Such treatment is effected by administering a complex of the present invention capable of trapping the pathogen to the subject. As used herein, the term “subject” refers to an animal, preferably a mammal such as a human.

The complex of the present invention can be administered to the subject per se, or in a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the complex accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients arc, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

Sustained-release (SR), extended-release (ER, XR, or XL), time-release or timed-release, controlled-release (CR), or continuous-release (CR or Contin) pills are tablets or capsules formulated to dissolve slowly and release a drug over time. Sustained-release tablets are formulated so that the active ingredient is embedded in a matrix of insoluble substance (e.g. acrylics, polysaccharides etc) such that the dissolving drug diffuses out through the holes in the matrix. In some SR formulations the matrix physically swells up to form a gel, so that the drug has first to dissolve in matrix, then exit through the outer surface.

Difference between controlled release and sustained release is that controlled release is perfectly zero order release that is, the drug releases with time irrespective of concentration. On the other hand, sustained release implies slow release of the drug over a time period. It may or may not be controlled release.

Pharmaceutical compositions suitable for use in the context of the present invention include compositions wherein the active ingredients are contained in an amount effective to achieve the intended purpose. More specifically, a “therapeutically effective amount” means an amount of active ingredients effective to prevent, alleviate, or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p.1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

Once administered to the subject, the scaffold will ‘filter’ the biological fluid, and the active moiety will trap or process the entity within the scaffold in a targeted or non-targeted fashion. Clearly, not all components present in the biological fluid will pass through the pores of the scaffold, for example, red blood cells are too large to fit through the pores, and thus will not pass through the pores of the scaffold.

When utilized in vivo, the complex of the present invention will naturally be cleared from the subject, for example by phagocytes or through the kidneys. Clearance from the body will likely be slowed down in cases where the complex of the invention is configured for minimizing immunogenicity.

The complex of the present invention can also form a part of a device which is positionable within a body vessel. For example, the complex can be incorporated into a container, such as a biocapsule (e.g. the Theracyte® immunoisolation device). For further description of biocapsules please see US 2010/0028398, or US 2011/0092949.

Such a capsule could be configured as a filtration device and placed (anchored) in a major blood vessel, or an organ. It may function like a filter grafted or implanted in the specific organ, such as in the spleen, liver, heart, kidney, lymph nodes, etc. Furthermore, the container may be free flowing in biological fluid (e.g. GI fluid or blood circulation).

The complex of the present invention can further include a moiety for capturing viruses. Such a moiety can be a virus-specific receptor, as defined herein above, an antibody, a ligand, a receptor, and the like. As mentioned previously, a non-exhaustive list of receptors that function as virus-specific includes CD4, CCR5, CXCR4, CCR2, CCR3, Tetraspanin CD81, human scavenger receptor SR-BI, Claudin-1, Occludin, Ephrin-B2, CD46, CAR, av integrin, HAVCR-1, EGFR (epidermal growth factor receptor), SLAM, acetylcholine receptors, neurotrophin receptor, p75 NTR, sialic acid, glycosaminoglycan and heparan sulfate.

The complex of the present invention can further include a moiety for capturing microorganisms. Such a moiety can be a host-specific moiety incorporated by a parasitic microorganism, such as a protein, a carbohydrate, a lipid, an antibody, a ligand, a receptor, and the like. A non-exhaustive list of proteins that function as host-specific moiety incorporated by a parasitic microorganism can include annexins (e.g. annexin A1, annexin A2, annexin A4, annexin A5, annexin A11), Tetraspanins (e.g. CD81, CD9), CD59, Cyclophilin, beta tubulin, cofilin 1, enolase 1, fatty acid synthase, gamma-glutamyltransferase 1, glypican 4, phosphoglycerate kinase, pyrovate kinase, S100 calcium-binding protein A11, topomyosin 1, transgelin

The complex of the present invention can also be used to remove pathogens or substances from a biological sample. For example, a blood or tissue sample (suspended in a buffer) can be passed through a device containing the complex of the invention. Pathogens or substances will ‘filter’ through the complex and be trapped and inactivated by the active moiety.

As is mentioned hereinabove, the present complex can also be used to purify a non-biological fluid from substances such as impurities, toxins etc. Such a fluid can be water of any source, beverages, liquid culture medium, sewage, blood samples, liquid waste, and the like.

As used herein, the term “impurities” refers to suspended particles, parasites, bacteria, algae, viruses, fungi, archaea, protist, organic debris, metals, nucleic acids, small molecules, prions, protocells, proteins, peptides, carbohydrates, lipids, toxins, venoms, drugs, poisons, allergens, or pollutants.

When applied ex vivo, particularly in non-living systems, the complex of the invention may be optionally cleared by centrifugation, precipitation, by means of chromatography, pulled by magnetic force, separated by electrophoresis, and the like. To facilitate magnetic separation, the scaffold can be modified using the approaches described by Yabin et al., [European Polymer Journal Volume 43, Issue 3, March 2007, Pages 762-772] or Liva et al.[Journal of Colloid and Interface Science, Volume 375, Issue 1, 1 Jun. 2012, Pages 70-77].

Thus, the complex of the invention can further include a tag such as a magnetic, fluorescent or luminescent tag for enabling purification of the complex from a liquid via, for example, FACS, a magnetic sorter and the like.

The complex of the present invention can be packaged as a kit which includes the complex provided as powder or suspension (in a vial), accompanying reagents for administering the complex (e.g. buffer etc), an administration device (e.g. syringe) and instructions for use.

Exemplary protocols for treating a human subject infected with HIV or Influenza are provided below.

Treatment of an HIV-Infected Subject with an Anti-HIV Complex
An anti-HIV complex is generated by constructing a hollow microsphere-shaped scaffold 1μm in diameter using DNA origami (see Examples section for further detail). The external surface of the DNA microsphere has pores of 300 nm and is PEGylated and a 500 nm (in diameter) liposome bearing CD4 and CCR5 receptors is trapped within the microsphere. The liposome is bound to the nucleic acid scaffold via an antibody.

### Treatment Protocol:

A dose containing 25,000,000 units of the anti-HIV complex in a saline carrier is delivered to a patient via slow-drip infusion (IV), dose is repeated or adjusted based on viral load.

### Treatment of an Influenza-Infected Subject

An anti-Influenza complex is generated by constructing an anti-viral dendrimer of sialic acid molecules within a scaffold formed from Polystyrene, crystalline silicon, PLGA or Chitosan. The scaffold has pores of 400 nm on every side and is PEGylated.

### Treatment Protocol:

A nasal spray including 10,000,000 trap units per 1 ml dose is used as a prophylactic or for treatment of an infection.

It will be appreciated that the present approach can also be used to treat pathogen infections of plants and other multi-cell organisms. For example, a virus trap can be injected directly into a tree (via trunk-injection) to be systemically distributed throughout the tree. Injections are made in the bottom 18 inches of the tree, at intervals of around 6 inches apart. The depth for the injection is between ⅝″ and 1⅝″ into the tree. A 10 inch diameter tree would receive approximately a 1.5 ounce injection.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Polystyrene Microsphere traps

A population of polystyrene (PS) microspheres was generated and qualified with respect to size distribution and pore size range.

### Materials and Methods

### Non-Porous Microspheres

A 500 ml tri-neck round flask was used to dissolve 3.75 gr of polyvinylpyrrolidone in a mixture of 150 ml ethanol and 62.5 ml methoxy-ethanol. The flask was supplemented with a condenser and a mechanical stirrer. Temperature of mixture was slowly raised, via oil bath, to 73° C., while nitrogen was bubbled thru the system. Separately, a 100 ml round flask was used to dissolve 1.5 gr benzyl-peroxide in 37.5 ml styrene, the resultant solution was stirred via magnetic stirrer, under nitrogen. Following temperature stabilization, the content of the 100 ml flask were poured into the 500 ml tri-neck round flask, and the reaction was left over-night.

Prior to washing, microspheres were visualizes under a light microscope to evaluate their size and homogeny.

The reaction mixture was evenly distributed into 12, 50 ml-vials and washed 6 times with ethanol to remove styrene residues followed by a single wash with 50% ethanol in water and two washes with water. Washing was done by spinning the vials in a centrifuge at 5500 rpm for 8 minutes, and slowly pouring the supernatant. Following washing, samples were lyophilized overnight.

### Porous Microspheres

Porous microspheres were prepared by modifying the procedure described by Omer-Mizrahi and Margel [Polymer Volume 51, Issue 6, 2010, Pages 1222-1230]. Briefly, 1 gram of lyophilized PS microspheres was suspended in 50 ml water and 1 ml of ethanol. The suspension was then sonicated for 8 minutes (35% amplitude) followed by ozonolysis for 20 minutes in a 100 ml round flask supplemented with a magnetic stirrer.

The resultant sample was washed with water (5500 rpm for 8 minutes) until no ozone was left in the supernatant, as measured by color change upon addition of sodium iodide.

The washed sample pellet was suspended in 9 ml water and evenly distributed into 9, 20 ml-scintillation vials, each vial containing 1 ml water, equivalent to ˜100 mg PS. Seven ml of 1.43% sodium dodecyl sulfate (SDS) were added followed by addition of Glycidyl-methacrylate (GMA) at varying volumes: 0.1 ml×2, 0.2 ml×2, 0.25 ml, 0.3 ml×2, 0.35 ml×2. Following addition of 14 mg of sodium bisulfite, each vial was stirred at room temperature overnight. The samples were then washed twice with 30% ethanol in water, twice with ethanol and once with 20% ethanol. Washing was carried out at 5500 rpm for 8 minute each time.

### Results

### Non-Porous Microspheres

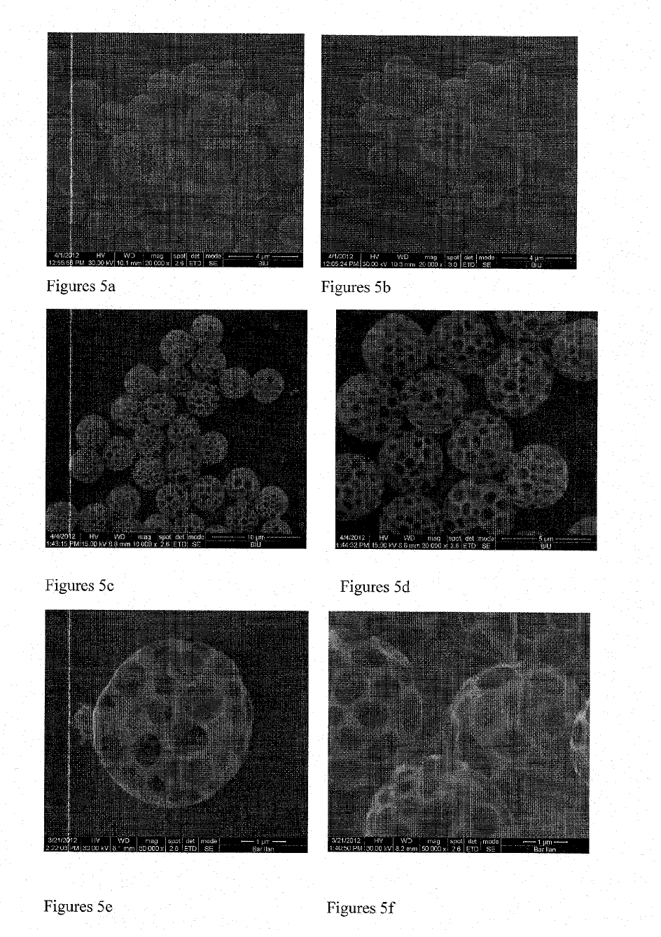

Visualized under a light microscope the polystyrene microsphere population appeared homogenous with a diameter of microspheres ranging between 2.1-2.2 μm (FIG. 4a). When visualized under a scanning electron microscope most of microspheres were ˜1.7 μm in diameter when dry and 2.2 μm when hydrated (FIGS. 4b-c).

### Porous-Microspheres

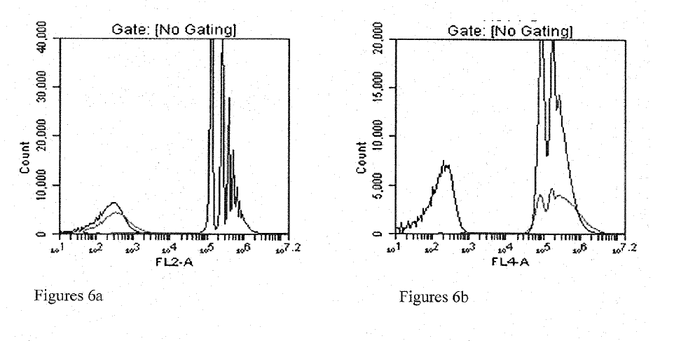

Reactions with GMA produced a uniform population of porous microspheres which swelled in size as a result of poration to a diameter ranging between 3.5 and 4.1 μm and a pore diameter ranging between 0.3 and 1.0 μm (FIGS. 5c-f). Lower volumes of GMA (0.5 ml) had no effect on the microspheres, increasing the GMA volume from 0.5 to 1.5 ml produced a uniform population of hollow hemispheres and degraded beads. Increasing ozonolysis time to 40 minutes produced a similar result.

### Discussion

The methodology described herein generated a uniform population of polystyrene microspheres. Pore generation using a combined approach of ozonolysis and GMA polymerization resulted in a narrow pore diameter range which would enable use of the microspheres as traps for baculoviruses which range in diameter between 50 and 250 nm

### Example 2

### Polystyrene Nanosphere Carriers

A population of polystyrene (PS) nanospheres was generated and qualified with respect to size distribution and pore size range.

### Materials and Methods

### 600 nm Polystyrene Beads

The methodology was adapted from Goodwin et al. [COLLOID & POLYMER SCIENCE Volume 252, Number 6 (1974), 464-471]. Briefly, 1 ml of styrene was added to 8.5 ml of a 2.5×10⁻³M sodium chloride solution followed by addition of 0.5 ml of a 0.05 M potassium persulfate solution. The resultant mixture was stirred in a 20 ml scintillation vial overnight at 73° C., followed by washing twice with water, twice with ethanol and twice with water. Washing was done by spinning the vials in a centrifuge (10000 rpm for 12 minutes) and slowly pouring the supernatant.

### 250 Nm Polystyrene Beads

In a 20 ml scintillation vial 5 ml of 1×10⁻³M sodium dodecyl sulfate (SDS) was mixed with 5 ml of a 7.1×10⁻³M 4,4′-azobis(4-cyanovaleric acid). pH was adjusted to 7.8 with NaOH and 0.75 ml of styrene were added followed by stirring at 73° C. overnight. The samples were washed via centrifuge spinning (11000 rpm for 20 minutes) and slowly pouring the supernatant.

### Results

### 600 Nm Polystyrene Beads

The polystyrene beads were analyzed for size using dynamic light scattering (DLS/Nanophox) and the resulting diameter was 591±77 nm.

### 250 nm Polystyrene Beads

The polystyrene beads were analyzed for size using dynamic light scattering (DLS/Nanophox) and the resulting diameter was 231±31 nm. Reducing styrene volume to 0.3 ml produced the same size of beads.

### Discussion

Two populations of nanospheres having an average diameter of 590 nm or 230 nm were generated. Both populations are capable of diffusing into the 4 micron polystyrene microspheres described in Example 1, which have pore diameters ranging between 300 nm and 1000 nm. By coating the nanospheres with active agents (e.g. virus binding moieties etc) and sequestering the nanospheres within the microspheres, one can generate an immuno-privileged anti-viral trap.

The polystyrene porous-microspheres form the outer surface of the trap which is inert to the body immune system while the coated nanospheres trapped inside the microspheres are shielded from the immune system and are capable of binding and immobilizing viruses that enter the trap via the pores of the microspheres.

### Example 3

### Baculovirus In Vitro Experiment

An Sf9 culture system was utilized to test the effectiveness of 4 μm polystyrene micro-particles coated with various surface active agents capable of binding and/or inactivating baculovirus virions.

### Materials and Methods

### Traps

Biotin-streptavidin binding was used in order to bind Cremophore, Polylysine, Amphotericin, Tween, Triton 100-×, Cathepsin, Surfactant Protein D or anti-gp64 (a specific antibody directed at baculovirus envelope protein) onto the porous polystyrene microspheres, thereby generating viral traps.

Rhodamine B was used as control to ensure proper binding. Bovine Serum Albomine (BSA) and anti-IL13 were used as negative controls, since they are not expected to interfere with viral infection.

### Binding Biotin to Active Agents

Thermo Scientific's EZ-link TFPA-PEG3-Biotin kit (product #21303, Mw=664 gr/mole) was used for covalent binding. The TFPA-biotin reagent is activated via UV light, and promotes binding of the biotin into C—H bonds existing in the active-agent compound. Each active agent was incubated with 0.1 mg of a biotin reagent, equivalent to 1.51×10⁻⁴ mmol. As specified in the kit protocol, a molar ratio of 10:1 biotin-reagent:active-agent is optimal for biotinylation. Accordingly, 1.51×10⁻⁵ of each active agent was used for biotin binding.

The following amounts of each active agent were used in the reaction:
0.34 mg of Polylysine: average Mw=22,500 gr/mole.
0.014 mg of Amphotericin: Mw=924 gr/mole.
0.0185 mg of Tween: Mw=1227.5 gr/mole (density is assumed to be 1 gr/ml).
9.8 μg of Triton 100-×: Mw=647 gr/mole (density is assumed to be 1 gr/ml).
2 μg of Cathepsin: Mw=28,900 gr/mole—equivalent to 6.9×10⁻⁵ μmole. To keep biotin reagent:active-agent ratio constant at 10:1, 0.46 μg of biotin-reagent were used.
0.045 mg of Cremophore: average Mw=3000 gr/mole (density is assumed to be 1 gr/ml).
0.045 mg of Surfactant protein D: Mw=65,000 gr/mole—equivalent to 3.1×10⁻⁵ μmole. To keep biotin reagent:active-agent ratio constant at 10:1, 0.21 μg of biotin-reagent were used.
7.2 μg of Rhodamine B: Mw=479 gr/mole.

The TFPA-biotin reagent was mixed with the active agents in PBS to final volume of 150 μl and incubated in the dark for 2 minutes, following which, solutions were incubated for 10 minutes, 5 cm below a 365 nm UV lamp (230V, 0.17 Amp, 39W). The control agents were covalently bound to biotin via Thermo Scientific's EZ-link sulfo-NHS-Biotin kit (product #21217, Mw=454.5 gr/mole). The reagent reacts rapidly with any primary amine-containing molecule to attach the biotin label via a stable amide bond. As specified in the kit protocol, a molar ratio of 10:1 biotin-reagent:active-agent is optimal for biotin binding.

The following amounts of each control agent were used in the reaction:
0.033 gr. of Bovine Serum Albumin: Mw=66,500 gr/mole. N=0.5 μmole. 2.3 mg of biotin-reagent were used.
10 μg of Anti-gp64 antibody: Mw=150,000 gr/mole. N=6.67×10⁻⁵ μmole. 0.3 μg of biotin-reagent were used.
10 μg of Anti-IL13 antibody: Mw=150,000 gr/mole. N=6.67×10⁻⁵ μmole. 0.3 μg of biotin-reagent were used.

### Allophycocyanin-Streptavidin (APC-Avidin) Coating of Porous PS Microspheres

A total of 50 million porous polystyrene microspheres were used for Avidin coating. Beads were washed three times with carbonate/bicarbonate pH=9.6 50 mM buffer and incubated with 20 μg APC-avidin (Mw=60,000 gr/mole, product #405207, BioLegend), corresponding to 3.33×10⁻⁴ μmole, in a 500 μl carbonate/bicarbonate buffer for 30 minute at RT using a shaker. The beads were then washed three times with PBS. Concentration of beads was measured via Accuri C6 flow cytometer, and aliquots of 4 million beads were prepared.

### Binding of Biotinylated Active Agents to APC-Avidin Coated Porous Polystyrene Microspheres

Each aliquot of APC-avidin coated porous polystyrene microspheres was mixed with the active agent biotinylation reaction mixture and incubated for 30 minutes at RT using a shaker. Each aliquot was subsequently washed 4 times with PBS. beads concentration was measured using Accuri C6 flow cytometer.

### Results

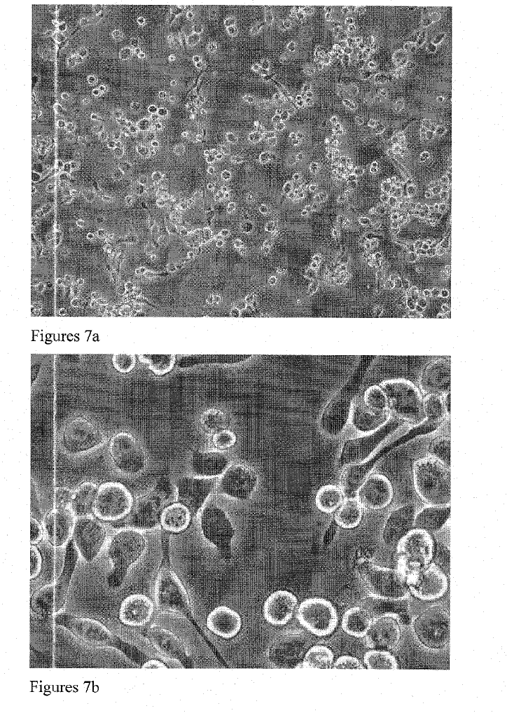

Flow cytometery readings (Accuri C6) of the porous polystyrene microspheres are shown in FIGS. 6a-b. FL2-A reading (FIG. 6a) corresponds to emissions at the yellow-orange spectra, while the FL4-A readings (FIG. 6b) correspond to emission at the far red spectra. Rhodamine B has a maximum emission at 625 nm, corresponding to readings at FL2-A. Allophycocyanin (APC) has a maximum emission at 661 nm, corresponding to readings at FL4-A. FIGS. 6a-b show the FL2-A and FL4-A readings of polystyrene beads (black), polystyrene coated with APC-avidin (red) and polystyrene coated with APC-avidin bound to Rhodamine B (blue). Polystyrene beads (black) have low intensity readings at both FL2 and FL4; Polystyrene beads coated with APC-avidin (red) have a low intensity reading at FL2 but high intensity reading at FL4; Polystyrene beads coated with APC-avidin bound the rhodamine b (blue) have high intensity reading at both FL2 and FL4. The flow cytometer readings indicate that APC-avidin coating of porous polystyrene microspheres and biotinylation of active agents, as indicated by biotinylation of Rhodamine B were successful.

### Cells

Sf9 cells were propagated at 27° C. in SFM921 serum-free insect cell culture medium (Expression Systems). Sf9 cells were grown either as monolayers in 12-well plates or in suspension in shaker flasks agitated at 130 rpm.

Competent E. coli DH10BAC cells, containing bacmid (baculovirus shuttle vector plasmid) and a helper plasmid, were used to generate recombinant bacmids according to the manufacturer's protocol (Invitrogen). Insertion of the gene (GFP) into the bacmid was verified by PCR. Sf9 cells were transfected with recombinant bacmid DNA using ESCORT transfection reagent (Sigma-Aldrich) in 6-well plates. The cells were incubated for 5 h at 27° C., rinsed and incubated for another 72 h. Media were harvested, centrifuged, and the virus containing supernatant was used for 2-3 successive infections resulting in amplification of the virions.

Sf9 cells (2*10⁵ cells/ml) were seeded on 12 well plates, 1 ml/well. Infection of cells was done at Multiplicity Of Infection (MOI) of 10 at two different protocols; pre-incubation and prophylactic. At pre-incubation mode, virus was incubated separately with the various traps for 30 minutes, centrifuged at 5000 g for 3 minutes and supernatant was added to the cells. At prophylactic mode, traps were added to the cells and only then virions were added too. Plates were placed in a humidified chamber placed at 27° C. for 48 hours, harvested by vigorous pipeting well content and analyzed for GFP fluorescence by FACS (C6 accuri).

### Results and Discussion

Viruses are well known for the damage they cause to an infected host organism. At present, there are no anti-viral agents that exhibit the effectiveness or multi-spectrum applicability of antibiotics.

The present invention presents a new approach for combating viral infections by providing viral traps that are capable of physically and chemically inactivating infectious agents such as viruses. The present invention also provides a model system for observing and quantifying the anti-viral activity of various viral trap configurations.

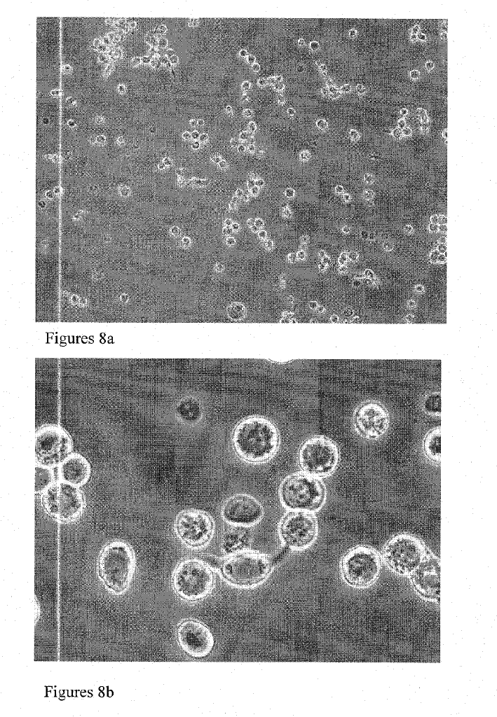

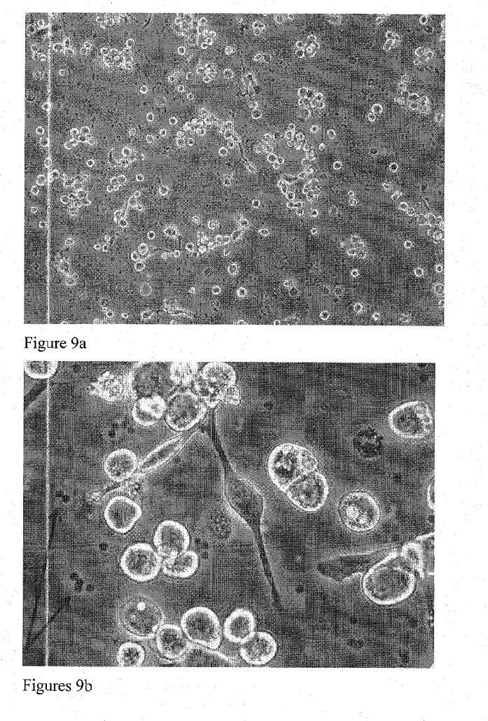

Cell culture experiments show an observable difference in the vitality of the healthy untreated Sf9 cells (FIGS. 7a and 7b) in comparison to infected Sf9 cells (FIGS. 8a and 8b). Co-treatment of the Sf9 cells both with viruses and porous polystyrene traps (FIGS. 9a and 9b-traps indicated by arrows) resulted in an observable rescue of infection by most of the Sf9 cells, which are qualitatively more similar to the healthy untreated cells than to the infected cells.

The rescue of baculovirus associated infection was further assessed quantitatively. As is shown in FIGS. 9c and 9d, the highest rate of infection inhibition is achieved by PS alone, Tween, Poly Lysine, Surfactant protein D and Triton.

### Example 4

### Baculovirus In Vivo Experiment

Blaberus craniifer cockroaches, also known as ‘death's head’ cockroaches, were used as an in vivo model to evaluate the effectiveness and toxicity of porous polystyrene microsphere traps.

### Materials and Methods

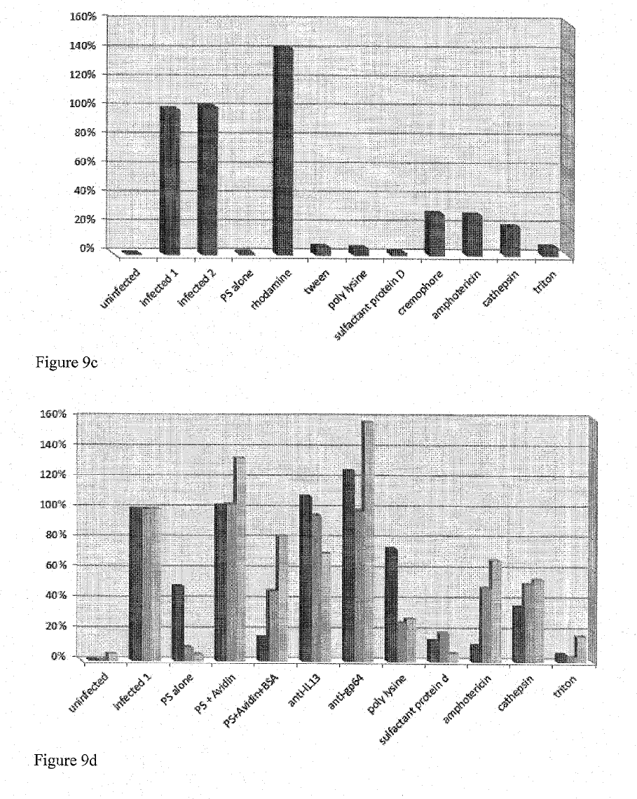

Porous polystyrene microsphere traps were mixed with Baculovirus and immediately injected into the abdominal hemocoel of cockroaches so that the needle enters between the third and fourth abdominal sternites, close to the lateral margin (FIG. 10a). A total of 10 μl were injected into each cockroach, comprising 1 μl of Baculovirus and the appropriate volume of traps, for a total of 165,000 traps particles per cockroach. Prior to injection, cockroaches were kept at −20° C. for 7 minutes to anesthetized them.

Traps used in the injection experiment are polystyrene particles covered with either Triton, Poly-lysine, Surfactant Protein D, Anti-GP64 antibody or Bovine Serum Albomine (BSA). Naked polystyrene particles were used as control. 2 cockroaches were injected with that same trap as duplicates, in addition, as positive control, 2 cockroaches were injected only with Baculovirus and as negative control, 2 cockroaches were not injected at all.

Hemolymph cells were harvested 5 days after injection by puncturing the membrane at the base of the metathoracic leg and gently squeezing the cockroach gently to release the hemolymph. 25 μl of hemolymph was quickly gathered by a micropippetor tip already containing 25 μl of ice-cold anticoagulant buffer (30 mM citric acid, 30 mM sodium citrate, 0.5M EDTA, 0.02% sodium azide) and suspended in an eppendorf containing 50 μl of ice-cold anticoagulant buffer. Cells were counted, and their relative GFP fluorescence was evaluated using Accuri C6 flow cytometer.

### Results:

The relative GFP fluorescence of hemolymph cells is a direct measurement of viral infection since baculovirus used in this experiment express a cytosolic GFP marker. Relative GFP fluorescence was compared to that of cockroaches injected with only Baculovirus.

### Discussion:

Traps did not produce any visible toxic effect on the cockroaches. Moreover, the injected traps reduced the infection-related GFP fluorescence (FIG. 10b). Traps coated by either Triton or Poly-Lysine produced results comparable to that of uninfected samples, suggesting a complete rescue of infection.

### Example 5

### Baculovirus Adhesion to Traps

Traps were incubated with viruses to show their capability to immobilize Baculovirus viral particles, thereby facilitating their inhibition.

### Materials and Methods

400,000 traps from each active-agent were gently mixed over 2′ with 10 μl of viruses. Each sample was then washed with 1 ml of PBS, and centrifuged for 8 minutes at 5 g. After removal of the supernatant, 1 ml of PBS was added and samples were gently mixed for 8 minutes, followed by centrifuges for 8 minutes at 5 g. 900 μl of supernatant were removed and pellet was suspended in the remaining 100 μl.

The traps had the following active-agents: Tween, Triton, Amphotericin, Surfactant Protein D. as control both naked porous microspheres and Avidin-coated porous microspheres were used.

### Results

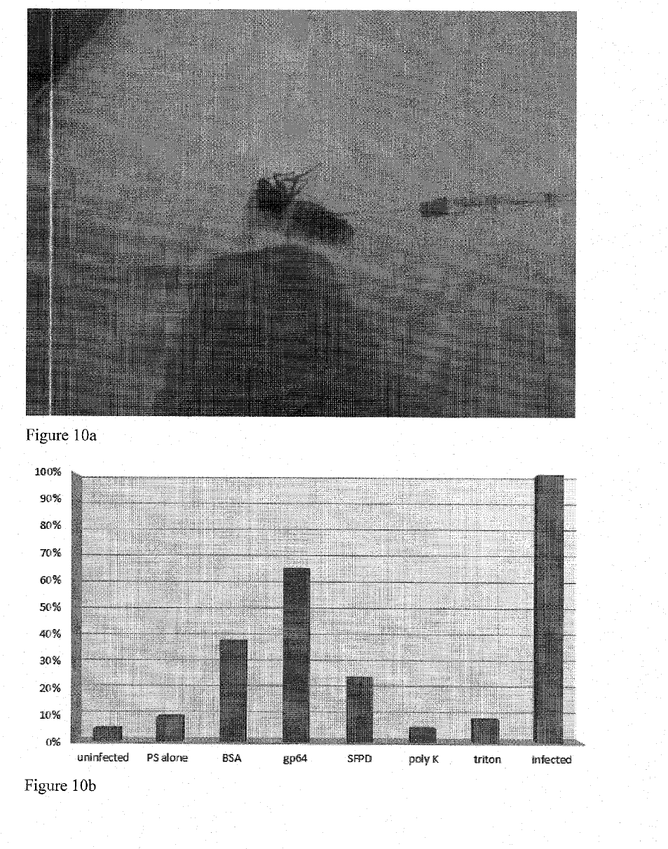

While the control traps, i.e. the naked porous microspheres and Avidin-coated porous microspheres showed only minimal binding of viral particles to their surface, traps containing Triton (FIGS. 11a-b), Tween and Amphotericin showed moderate binding of viral particles, as evident by SEM.

SEM was carried out using carbon coating on a glass surface. Acceleration voltage was 3 kV. Working distance was 2.6-2.9 mm. Bars are indicated for each photo. The particles adhering to the surface of the traps are at the expected size range of Baculovirus virions, 50-300 nm

### Example 6

### DNA Origami Scaffolds

The design of the DNA origami-based scaffold of the invention was developed with the help of caDNAno, a graphical-interface-based computer-aided-design environment created to assist in the generation of honeycomb-pleated origami designs [Douglas et al. (2009) Nucleic Acids Research, first published online doi:10.1093/nar/gkp436].

DNA origami-based scaffolds are prepared by combining 20 nM scaffold DNA, with 100 nM of each staple oligonucleotide, buffer and salts including 5 mM Tris, 1 mM EDTA (pH 7.9 at 20° C.), and 22 mM or 15 mM MgCl₂. Folding is carried out by rapid heat denaturation followed by slow cooling from 80 to 61° C. over 80 min, then 60 to 24° C. over 173 h. For visualizing the resulting products, a sample is electrophoresed on 2% agarose gels (0.5×TBE, 11 mM MgCl₂, 0.5 mg/ml ethidium bromide) at 70V for 4 h in an ice-water bath.

Essentially any purified arbitrary and high-complexity (not repeats) ssDNA could serve as a scaffold for origami DNA. Examples of sequences that may be used are M13mp18 (SEQ.ID.NO.1), p7308 (SEQ.ID.NO.2), p7560 (SEQ.ID.NO.3), p7560 old (SEQ.ID.NO.4), p7560 lab (SEQ.ID.NO.5), p7560 antisense (SEQ.ID.NO.6), p7704 (SEQ.ID.NO.7), p7704 lab (SEQ.ID.NO.8), p8064 (SEQ.ID.NO.9), p8064 lab (SEQ.ID.NO.10), p8100 (SEQ.ID.NO.11), p8100a (SEQ.ID.NO.12), p8100b (SEQ.ID.NO.13), p8100c (SEQ.ID.NO.14), p8634 (SEQ.ID.NO.15), pEGFP (SEQ.ID.NO.16).

DNA sequences used as staples may be, for example, as provided in the caDNAno gallery website [http://cadnano.org./gallery.html], and in Table 1 below.

TABLE 1

| | | SEQ.ID.NO. |
|---|---|---|
| | Length | |
| Icosahedron monomer A core sequences | (nt) | |
| CTTTATTCGTAAAACTAGCATATTCAACCGTTCTA | 35 | 17 |
| AGGGTGAGAAAGGCAAGAATTAGCAAAAATCGGTT | 35 | 18 |
| GCTGATAAATTAATGAGCATAAAGCTAATTAAGCAAT | 42 | 19 |
| AAAGC | | |
| CGATGAACGGTAATTCAACGCAAGGATAGGTAAAGAT | 42 | 20 |
| TCAAA | | |
| CTAATTGGGCTTGACTGAGTAATGTGTAAAAATTTTTA | 42 | 21 |
| GAAC | | |
| GAACGAGTAGTACAGCCGGAGAGGGTAGGCAAACAA | 42 | 22 |
| GAGAAT | | |
| CCTCATATATTTGACCTGAGAGTCTGGACTATTTTTGA | 42 | 23 |
| GAGA | | |
| AATAAATGCAATGCGATGGTTTAATTTCGACGAGAAA | 42 | 24 |
| CACCA | | |
| TCTACAAAGGCTACATAAGGCTTGCCCTAACTTTAATC | 42 | 25 |
| ATTG | | |
| GTATCAGGTCATTGGGACAGATGAACGGCTGACCAAC | 42 | 26 |
| TTTGA | | |
| TGAATTACCTTATGATAAGGGAACCGAATGTACAGAC | 42 | 27 |
| CAGGC | | |
| TGCTCATTCAGTGAAACGGAGATTTGTAAAGCGCGAA | 42 | 28 |
| ACAAA | | |
| GCATAGGCTGGCTGCCAGCGATTATACCTCATCGCCTG | 42 | 29 |
| ATAA | | |
| GCAGACGGTCAATCCGATTTTAAGAACTCAACGTAAC | 42 | 30 |
| AAAGC | | |
| ATTGTGTCGAAATCTTCATTACCCAAATGGCTCAT | 35 | 31 |
| TTGACCCACCTTCATCAAGAGAGCCGGAACGAGGC | 35 | 32 |
| AGATACACCTTATTAGCGTTTAAGGCCGGAAACGT | 35 | 33 |
| GAATTAGAGCCAGCAAAAATCTACGTTAGGTAGAA | 35 | 34 |
| CACCAATGAAACCAACAACATTATTACAATAAAACGA | 42 | 35 |
| ACTAA | | |
| TTCGGTCATAGCCCTAACGCCAAAAGGACTTGAGCCA | 42 | 36 |
| TTTGG | | |
| CGTAAACAGTTCAGATCACCGTCACCGAATTACGAGG | 42 | 37 |
| CATAG | | |
| CCTCAAATGCTTGATCGATAGCAGCACCTTTTCATCGG | 42 | 38 |
| CATT | | |
| TAAGAGCAACACTACAGACTGTAGCGCGGTAATCAGT | 42 | 39 |
| AGCGA | | |
| ATTAAAGGTGAATTAAAACGAGAATGACATTCATTGA | 42 | 40 |
| ATCCC | | |
| CAGAATCAAGTTCAGAATCGTCATAAATCATAAATCA | 42 | 41 |
| AAAAT | | |
| ACTGCCTTTAGCGTTCATAACCCTCGTTGACGGAAATT | 42 | 42 |
| ATTC | | |
| CAGGTCTTTACCCTGGAAGGTAAATATTTACCAGACGA | 42 | 43 |
| CGAT | | |
| CGTCCAATACTGCGCGGAATAAGTTTATAGAAACGCA | 42 | 44 |
| AAGAC | | |
| AAAAACCAAAATAGTGGCAACATATAAATTTGTCACA | 42 | 45 |
| ATCAA | | |
| ACCGATTGAGGGAGGACTATTATAGTCATTTAGACTG | 42 | 46 |
| GATAG | | |
| TAGAAAATTCATATGTAATAGTAAAATGGAAGCAA | 35 | 47 |
| ATAAAGGCGAGAGGCTTTTGCAAGGGCGACATTCA | 35 | 48 |
| CTTTAATAGAGTCAATAGTGAGAAACAGTACATAA | 35 | 49 |
| ATTAATTACATTTAGAGGAAGCCCGAAAAAAGCGA | 35 | 50 |
| ATCAATATATGTGATTAATTCGAGCTTCGACTTCAAAT | 42 | 51 |
| ATCG | | |
| TTAAGACGCTGAGATGCTCCTTTTGATAATCAAGAAAA | 42 | 52 |
| CAAA | | |
| CGGTTTAGCTATATATGATGAAACAAACAGAGGTCAT | 42 | 53 |
| TTTTG | | |
| GGTCAATAACCTTTGTGAATAACCTTGCAGCGATAGCT | 42 | 54 |
| TAGA | | |
| CGGATGGCTTAGAGATCCTTGAAAACATTTCTGTAAAT | 42 | 55 |
| CGTC | | |
| CTGAGCAAAAGAAGTTTCATTTGGGGCGTACATTTCGC | 42 | 56 |
| AAAT | | |
| GCTATTAATTAAACGTTTGACCATTAGACGAGCTGAAA | 42 | 57 |
| AGGT | | |
| TATTTTCCCTTAGACTTAATTGCTGAATTTCATTTCAAT | 42 | 58 |
| TAC | | |
| GGCATCAATTCTACGCAGAGGCGAATTAATAATGCTG | 42 | 59 |
| TACCT | | |
| AACGAGTAGATTTAGTCAGATGAATATATAGATTTTCA | 42 | 60 |
| GGTT | | |
| CAACATGTTTTAAAATAAAGAAATTGCGCAGTAACAG | 42 | 61 |
| TACCT | | |
| AGTTACAAAATCGCTAATAGTAGTAGCATTCCCAATTC | 42 | 62 |
| TGCG | | |
| TTTACATCGGGAGACATATAACAGTTGATTAACAT | 35 | 63 |
| AACAGAATATGCAACTAAAGTTGCTTTGAATACCA | 35 | 64 |
| GTCTCGTGGGTTAGAACCTACCATTTTGCGGAACA | 35 | 65 |
| CTTTGCCCGAACGTAAATCCCGTAAAAATAAAGTT | 35 | 66 |
| AAGAAACCACCAGATTAGTGATGAAGGGAAGCCGCAC | 42 | 67 |
| AGGCG | | |
| TCTGAATAATGGAACGCTGGCAGCCTCCACTCGTATTA | 42 | 68 |
| AATC | | |
| GCGCCGGGCGCGGTCAAACAATTCGACAGGCCAGAGC | 42 | 69 |
| ACATC | | |
| CGCACTCAATCCCTAGGAGCGGAATTATGTTTGGATTA | 42 | 70 |
| TACT | | |
| CTCATAACGGAACGATATAATCCTGATTCATCATATTC | 42 | 71 |
| CTGA | | |
| AGTATTAGACTTTATGCGGTATGAGCCGATTGCAGGCG | 42 | 72 |
| CTTT | | |
| TTATCAGATGATATGCATCAGCGGGGTCGGTCACTGTT | 42 | 73 |
| GCCC | | |
| ATGGCAATTCATCATGCCGGACTTGTAGTTTGAGGATT | 42 | 74 |
| TAGA | | |
| TGCGGCTGGTAATGCAATAGATAATACAAACGTCAGC | 42 | 75 |
| GTGGT | | |
| GCTGGAGGTGTCCACTGGTCAGTTGGCACAAACCCTC | 42 | 76 |
| AATCA | | |
| GCTGGTCTGGTCAGTGAACCTCAAATATAATCAACAGT | 42 | 77 |
| TGAA | | |
| TAGATTAGAGCCGTGGTAAAGGTTTCTTGCAACCAGCT | 42 | 78 |
| TACG | | |
| AGGAATTGAGGAAGTGGTGCCATCCCACTGCTCGT | 35 | 79 |
| ACCTTGCCAGCAACCGCAAGAGCACTAACAACTAA | 35 | 80 |
| ATTATTTCGCTGAGAGCCAGCTAGTCTTTAATGCG | 35 | 81 |
| AAGCGTAAGAATACTGCTGGTAATATCCAAAAACG | 35 | 82 |
| CGAACTGATAGCCCGCCATTGCAACAGGAGAACAATA | 42 | 83 |
| TTACC | | |
| CTGCAACAGTGCCAACATTGGCAGATTCCCCTTCTGAC | 42 | 84 |
| CTGA | | |
| GCAATTTAGGCAGACAACAGAGATAGAAACCAGTCAC | 42 | 85 |
| ACGAC | | |
| ACGCCAACATGTCATAAAACATCGCCATAGTATTAAC | 42 | 86 |
| ACCGC | | |
| CAGTAATAAAAGTGGAGGTGAGGCGGTCTAAAAATAC | 42 | 87 |
| CGAAC | | |
| CGGGACATTCTGGCGGCATTTTCGAGCCCGCCATATTT | 42 | 88 |
| AACA | | |
| GAACCACCAGCAATGCTTAATTGAGAATAGTAATAAG | 42 | 89 |
| AGAAT | | |
| ATGAAGATAAAACATGATAAATAAGGCGTTTGAAATA | 42 | 90 |
| CCGAC | | |
| ATAAAGTACCGACACCTAAATTTAATGGTTAAATAAG | 42 | 91 |
| AATAA | | |
| CGCTCAACAGTAGGAACTATATGTAAATGCTTAGGTTG | 42 | 92 |
| GGTT | | |
| ACACCGGAATCATACTTTTTAACCTCCGGCTGATGCAA | 42 | 93 |
| ATCC | | |
| ATTTCATCTTCTGAAAAGGTAAAGTAATCAGTATAAAG | 42 | 94 |
| CCAA | | |
| AATCGCAAGACAAAATACAAATTCTTACTCTGTCC | 35 | 95 |
| AGACTACATTACTAGAAAAAGATATATTTTAGTTA | 35 | 96 |
| AACAGCTAACGAAAGAGGCAAAGACAGCATCGGAA | 35 | 97 |
| GAGTTAAAGGCCGCAATAATTTTTTCACGAGCCTT | 35 | 98 |
| CGAGGGTAGCAACGAAAGGCTCCAAAAGGTTGAAAAT | 42 | 99 |
| CTCCA | | |
| AGGCACCAACCTAATGATACCGATAGTTCTGAGGCTT | 42 | 100 |
| GCAGG | | |
| AACCACCCCTATTATTCTGAACAGTAAGCGTCATA | 35 | 101 |
| CATTAAAGCCAGAATCACCGGAACCAGACACCCTC | 35 | 102 |
| CATGGCTTTTGATGTCCCTCAGAGCCGCGCCACCACCG | 42 | 103 |
| GAAC | | |
| TGCCTATTTCGGAAACCAGAGCCGCCGCAACAAATAA | 42 | 104 |
| ATCCT | | |
| CAATAATATCCTGAATCTTACGATATAGAAGGCTT | 35 | 105 |
| TTTCATCGTAGGAATCAGCTAATGCAGAGAACAAG | 35 | 106 |
| ATCCGGTATTCTAAAATAGATAAGTCCTACGCGCCTGT | 42 | 107 |
| TTAT | | |
| CCAGCTACAATTTTCGGCTGTCTTTCCTCAAGCCGTTTT | 42 | 108 |
| TAT | | |
| CACCGCCGTTTTTTGGGGTCGGTTGAGTGTTGTTC | 35 | 109 |
| ATCGGCAAAATCCCATGAATCGGCCAACCAGGGTG | 35 | 110 |
| CAGTTTGGAACAAGTGCGTATTGGGCGCGCGCGGGGA | 42 | 111 |
| GAGGC | | |
| ATCACCCAAATCAATGGCCCTGAGAGAGATGGTGGTT | 42 | 112 |
| CCGAA | | |
| CGACGTTCCAGCAGTTGGGCGCGCCATGTTTACCA | 35 | 113 |
| CGGGAACGGATAACGCGATCGGTGCGGGGGATGTG | 35 | 114 |
| GTCCCGGAATTTGTAGCTGGCGAAAGGGCCTCTTCGCT | 42 | 115 |
| ATTA | | |
| CTGCTCATTTGCCGGTAAAACGACGGCCAGGTGGAGC | 42 | 116 |
| CGCCA | | |
| AACTTTCAACAGTTCCCTCAGAACCGCCCCTCATTTTC | 42 | 117 |
| AGGG | | |
| CACAGACAGCCCTCCAGTACCAGGCGGAGATTAGCGG | 42 | 118 |
| GGTTT | | |
| ATAGCAAGCCCAATAAGAGAAGGATTAGTAAGTGCCG | 42 | 119 |
| TCGAG | | |
| GTTTAGTACCGCCATCAGCGGAGTGAGACGCCTGTAG | 42 | 120 |
| CATTC | | |
| AGGGTTGATATAAGAGTACAAACTACAAATAGAAA | 35 | 121 |
| ACTCCTCAGGAACCCATGTACACCGTACTCAGGAG | 35 | 122 |
| AGAACTGGCATGATCGCATTAGACGGGAGGTAATTGA | 42 | 123 |
| GCGCT | | |
| CTTACCGAAGCCCTTTATCCCAATCCAAAATAAACAGC | 42 | 124 |
| CATA | | |
| AATATCAGAGAGATTTTGCCAGTTACAAATAAGAAAC | 42 | 125 |
| GATTT | | |
| TAAAAACAGGGAAGTAAGACTCCTTATTATAGCAATA | 42 | 126 |
| GCTAT | 42 | |
| TTTGTTTAACGTCACAAGAAACAATGAAACGCAGT | 35 | 127 |
| AGCCTAAAACCCACAAGAATTCAGAGAGAATAACA | 35 | 128 |
| ACTTCTTTGATTAGCACCACACCCGCCGGGTTGCTTTG | 42 | 129 |
| ACGA | | |
| ACGGTACGCCAGAAAAGCCGGCGAACGTTTTAGAGCT | 42 | 130 |
| TGACG | | |
| GCACGTATAACGTGAAGGGAGCCCCCGAGGCGAGAAA | 42 | 131 |
| GGAAG | | |
| ACGCTGCGCGTAACTAATAACATCACTTGATTTTAGAC | 42 | 132 |
| AGGA | | |
| GGAAGAAAGCGAAAAGGCCGATTAAAGGGCCTGAG | 35 | 133 |
| AACCCTACTTTCCTCGTTAGACAAGTGTAGCGGTC | 35 | 134 |
| ATTGCGTTGCGCTCGGGCCGTTTTCACGCTGTTCTTCG | 42 | 135 |
| CGTC | | |
| AAATTGTTATCCGCAGCCAGCGGTGCCGAGATGCCGG | 42 | 136 |
| GTTAC | | |
| CGTGAGCCTCCTCATCGTTAACGGCATCGTGCCCCCTG | 42 | 137 |
| CATC | | |
| GGCCAGAATGCGGCACTGCCCGCTTTCCCTGTTTCCTG | 42 | 138 |
| TGTG | | |
| AGACGATCCAGCGCAATCATGGTCATAGAGTCGGG | 35 | 139 |
| CAGCAAACAGTTGAGGATCCCCTCTGTGGTGCTGC | 35 | 140 |
| GGTGCCGGAAACCAAAAAATAATTCGCGTTAAATGTG | 42 | 141 |
| AGCGA | | |
| CGCATCGTAACCGTAAATATTTAAATTGCAGGAAGATT | 42 | 142 |
| GTAT | | |
| GTAACAACCCGTCGAAAAGCCCCAAAAATAAACGTTA | 42 | 143 |
| ATATT | | |
| ATAGGAACGCCATCGGCAAAGCGCCATTTTGGTGTAG | 42 | 144 |
| ATGGG | | |
| TTGTTAAAATTCCICTGGGATAGGTCACGCGCCATT | 35 | 145 |
| TAATCAGGATTCTCCGTGGGATCATTTTTTAACCA | 35 | 146 |
| Icosahedron monomer A non-crossover vertex staple | | |
| sequences | Length | |
| AGATTCATCAGTTGAGTTTTTTTTTTTTACCATTAGCGC | 46 | 147 |
| CATCTTT | | |
| TATACCAGTCAGTTTTTTTTTGGATACGCGATTTTTTTT | 52 | 148 |
| TAAACACTCATCT | | |
| TAATTGTATCGGTTTATTTTTTTTTTTTAGCAGCGAAAA | 46 | 149 |
| GAATACA | | |
| GGAACAACTAAATTTTTTTTTTCACCTATAGTTTTTTTT | 52 | 150 |
| TTAAGAGGCTGAG | | |
| AGAACCGCCACCCTCATTTTTTTTTTTTTTACCGTTCAC | 46 | 151 |
| ATGAAAG | | |
| AAAAATAATATCCCATTITTTTTTTTTTAGCAAATCAC | 46 | 152 |
| AACGCTAA | | |
| AGACGACGACAATTTTTTTTTGCGTTGAACGTTTTTTTT | 52 | 153 |
| TCATAGGTCTGAG | | |
| ACCAGACCGGAAGCAATTTTTTTTTTTTTTTITAATGAT | 46 | 154 |
| TTATCAA | | |
| AGCGGATTGCATTTTTTTTTTAGGGGGGTTTTTTTTTTT | 52 | 155 |
| TGAAAATACATAC | | |
| ATGTTAGCAAACTTTTTTTTTAAGAGAAAATTTTTTTTT | 52 | 156 |
| TGCGTCTTTCCAG | | |
| CTGCAAGGCGATTAAGTTTTTTTTTTTTAACGTACAGG | 46 | 157 |
| TTGTGTAC | | |
| CAGGCTGCGCAATTTTTTTTTCGTAAATTAATTTTTTTT | 52 | 158 |
| TTACCCCGGTTGA | | |
| GTACCAAAAACATTATTTTTTTTTTTTTCAATATGATGT | 46 | 159 |
| CAATCAT | | |
| CCAATAAATCATTTTTTTTTTCATTCAACAATTTTTTTT | 52 | 160 |
| TATTTGCACGTAA | | |
| AAACGATGCTGATTGCTTTTTTTTTTTTTAACATTATCA | 46 | 161 |
| TATCAAA | | |
| CTCATGGAAATACCTATTTTTTTTTTTTAATGGCTATAG | 46 | 162 |
| CAAATGA | | |
| TAGAAGAACTCATTTTTTTTTACAGGGGAGCTTTTTTTT | 52 | 163 |
| TGCACTAAATCGG | | |
| GTTTTTCTTTTCACCATTTTTTTTTTTTCGAGATAGGAG | 46 | 164 |
| GTGCCGT | | |
| AAACCTGTCGTGTTTTTTTTTTTCGTAGTGTTTTTTTTTT | 52 | 165 |
| CACTGGTGTGTT | | |
| CATAAACATCCCTTTTTTTTTGTCGGGTTATTTTTTTTTT | 52 | 166 |
| ATCTAAAGCATC | | |
| Icosahedron monomer A crossover vertex staple sequences | Length | |
| GTTGGGAAGAAAATCACCTTTTTACTTTAATCTTTTCG | 52 | 167 |
| AGGTGAATTTCTTA | | |
| ATTGCGAATTTTTGCGGGTTTTGTATCCGTAATTTTTCA | 52 | 168 |
| GAGCCGCCACCAG | | |
| TAATCAAAATGGAAAGCGTTTTCACATTCAACTAATGC | 38 | 169 |
| ACAACATGTTCATTACCGTTTTTTCAACCTGTTTTTCAG | 52 | 170 |
| GATTAGAGAGTAC | | |
| AAAGATTAAACAATTTCATTTTGACAAAAAAG | 32 | 171 |
| CTTTAGAGTTTTTTGCATGTAGAAACCAAT | 30 | 172 |
| TTGGGAAGGCTCACCGGATTTTTCAGCACAAATTTTCT | 52 | 173 |
| TTTGCGGGAGAAGC | | |
| GGCAAGGCACGGAGACAGTTTTCTGATACGGTTTTTCG | 52 | 174 |
| CGGTCCGTTTTTTC | | |
| GACATAAAATATTAATTTTTTTAGGGTTTTCCCAGTCA | 38 | 175 |
| TATCGGCCTGTGGCACAGTTTTGCTGGATCAGTTTTAC | 52 | 176 |
| AGCTGATTGCCCTT | | |
| GCTGCATTATTATAAATCTTTTGTGCACGGGT | 32 | 177 |
| TAGGAATGCCTTTTCAATCGTCTGAAATGG | 30 | 178 |
| Icosahedron monomer A connector staple sequences | Length | |
| AATTGTAGCAATACGATATATTCGGTCGGCGCCGACA | 42 | 179 |
| ATGAC | | |
| GCAAATTAACCGAAGCTACAGAGGCTTTTAATGCCAC | 42 | 180 |
| TACGA | | |
| AACAACCATCGCGCACGGGTAAAATACGGAGGACTAA | 42 | 181 |
| AGACT | | |
| CGACATCCCTTACACCTTGATATTCACACAGCATTGAC | 42 | 182 |
| AGGA | | |
| TGCTCGTCATAACCATACAGGAGTGTACCAGTTAATGC | 42 | 183 |
| CCCC | | |
| GGTTGAGGCAGGTGAGTGCCCGTATAAATGGTAATAA | 42 | 184 |
| GTTTT | | |
| CATTATCAAAATCATCATCGAGAACAAGTATCATTCCA | 42 | 185 |
| AGAA | | |
| TCAATAGTGAATACGAACGCGAGGCGTTTTGCTATTTT | 42 | 186 |
| GCAC | | |
| CGGGTATTAAACTCTAAATCAAGATTAGTTAGCGAAC | 42 | 187 |
| CTCCC | | |
| GGTCCTTATTACGCGAAAATCCTGTTTGTTGCAGCAAG | 42 | 188 |
| CGGT | | |
| CATGATTAAGACTTAGTCCACTATTAAACCACTACGTG | 42 | 189 |
| AACC | | |
| CCACGCTGGTTTGTATCAGGGCGATGGCGAACGTGGA | 42 | 190 |
| CTCCA | | |
| CGCGATGAACGGTACGGCGTAAATTCAGAGTGCCAAG | 42 | 191 |
| CTTAC | | |
| AAACAAGAGAATCCGAGAGATAGACTTTTCAAACTTA | 42 | 192 |
| AATTT | | |
| TTCATGCTTCGGAAGCAGAAACAGCGGACTCCGTGGT | 42 | 193 |
| GAAGG | | |
| CAGTATTAAATCCTGAATTTTCTGTATGGATCTAAAGT | 42 | 194 |
| TTTG | | |
| TCCTGATTGTTTCTATAGTTAGCGTAACGGATTTTGCT | 42 | 195 |
| AAAC | | |
| TGGGATTATACTTCCTCAGAGCCACCACACCCTCAGAA | 42 | 196 |
| CCGC | | |
| CCGGTACCGAGCTCGAAACGCAATAATAAGCAGATAG | 42 | 197 |
| CCGAA | | |
| TAATTGCGTTGCATTTTTAAGAAAAGTAACGGAATACC | 42 | 198 |
| CAAA | | |
| TTGCTCACTGCCCGGAACAAAGTCAGAGGAATTAACT | 42 | 199 |
| GAACA | | |
| CTTGTTTAGTATCACATCACGCAAATTATTTATAATCA | 42 | 200 |
| GTGA | | |
| ATATAAAGTACCGATCCTGAGAAGTGTTACCGTTGTAG | 42 | 201 |
| CAAT | | |
| GGGACAAAAGGTAAAGGGCGCGTACTATCGCTTAATG | 42 | 202 |
| CGCCG | | |
| TTCAACATGTTTTATGCCTAATGAGTGAAACATACGAG | 42 | 203 |
| CCGG | | |
| ATATTTTCATTTGCTCACAATTCCACACGCTAACTCAC | 42 | 204 |
| ATTA | | |
| CTGGGGCGCGAGCTGCCAGCACGCGTGCGTCATACCG | 42 | 205 |
| GGGGT | | |
| AGTGGTTCCGAAATCTCCAGCCAGCTTTGAGGGGACG | 42 | 206 |
| ACGAC | | |
| GCGATGGCCCACGCGCATCTGCCAGTTTCCGGCACCGC | 42 | 207 |
| TTCT | | |
| AATACGTGAACCATAGCTTTCATCAACATCTGCTCCTTC | 42 | 208 |
| CTGT | | |

### Example 7

### Liposomal Active Moieties

Liposomes are prepared by the extrusion technique. Cholesterol (Chol) and 1,2-disteoroyl-sn-glycero-3-phosphatidylcholine (DSPC) are dissolved in ethanol free chloroform to approximately 10 mg/ml. Chloroform is removed while purging the sample under a stream of nitrogen gas until the samples become gel like. Subsequently, the samples are placed under a high vacuum where the lipid sample forms a “puffy” film that is kept under vacuum for 2-3 h to remove any residual chloroform. The samples are preserved hydrated at temperatures above their phase transition temperature (Tm).

The dried lipid is gently resuspended in 120 mM phosphate buffered saline, pH 7.4 (or other buffer with similar ionic strength and pH), to give a final lipid concentration of 20 mM.

Lipid suspension is shook for 30 minutes and then sonicated in a bath sonicator for 2 minutes. Hydration of liposomes is for 1 h with vigorous shaking or mixing.

The lipid suspension is passed at least 15 times through a 50 nm polycarbonate filter using an extrusion apparatus (available from Avestin or Avanti Polar Lipids, Inc.). Unilamellar vesicles are obtained.

The lipid suspension is purified by ultracentrifugation for 30 minutes at 15° C. at 100,000 g (e.g., 72,000 rpm on a Beckman TL-100ultracentrifuge). Purified small unilamellar vesicles with a mean diameter of 25 nm will be in the upper layer of the suspension. The lipid layer is transferred using a Pasteur pipette, to a new tube and stored at 4° C. under N₂ (g).

### Example 8

### Protocell Active Moieties

Protocells are prepared by fusing a mesoporous silica particle core with unilamellar lipid vesicles.

A precursor solution is synthesized by the addition of a non-ionic surfactant Brij-58 (CH₃(CH₂)₁₅—(OCH₂CH₂)20-OH, Aldrich) to an acidic silica sol (A2**). Tetraethyl orthosilicate (TEOS, Aldrich), ethanol, deionized water, and dilute HCl (mole ratios 1:3.8:1:0.0005) are reflux at 60° C. for 90 min to provide the stock sol. Then, 10 mL of stock sol is diluted with ethanol, followed by addition of water, dilute HCl, and aqueous surfactant solution (1.5 g of surfactant dissolved in 20 mL of water) to provide final overall TEOS/ethanol/H2O/HCl/surfactant molar ratios of 1:22:55:0.0053:0.06. This sol is stirred for about 10 min before beginning a powder synthesis run.

Monodisperse droplets are generated by means of a vibrating orifice aerosol generator (TSI model 3450). The solution is forced through a small orifice (20 μm diameter) by a syringe pump, with syringe velocities of approximately 8×10-4 cm/s (□4.7×10-3 cm³/s). This delivery rate is adjusted to provide a stable operating pressure of 340-420 kPa. The liquid stream is dispersed into uniform droplets by the vibrating orifice using a frequency range of 40-200 kHz, with the final setting adjusted to eliminate satellite droplets. The droplets are then injected axially along the center of a turbulent air jet to disperse the droplets and to prevent coagulation. Following the mixing of the dispersed droplets with a much larger volume of filtered dry air, the droplet-laden gas stream flowed through a 2.5 cm diameter quartz tube into a three-zone furnace (0.9 m heated length) maintained at 500° C. (A2** runs) or 420° C. (TEOS solution runs). The particles are collected on a filter maintained at approximately 80° C. by a heating tape. Collected particles are calcinated in air at 400-450° C. for 4 h to remove the surfactant template. Prior to use in further experimentation, the porous beads are washed in deionized water.

### Unilamellar Lipid Vesicles

Palmitoyl-oleoyl-phosphatidyl-choline dissolved in chloroform (to 10 mg/mL) is mixed with 5% of 1 mg/mL 1,2-dioleoyl-sn-glycero-3-{[N(5-amino-1-carboxypcntyl)iminodiacetic acid] (DOGS-NTA-Ni, Avanti Polar Lipids, Alabaster, Ala.) in a glass vial. The solvent is evaporated using a nitrogen stream until dry and the vial is kept under vacuum for 1 h to 2 h to remove residual solvent. The lipid film is hydrated using deionized (DI) water overnight at 4° C. The final lipid concentration is 2 mg/mL. The hydrated lipids are vortexed for several minutes and are extruded using a mini-extruder (Avanti Polar Lipids) with 0.1 mm polycarbonate membrane filters (Whatman, Inc., Newton, Mass.). The lipid solution is passed through the extruder at least 19 times and diluted with PBS to 1 mg/mL.

### Lipid Coating of Protocells

A 1 mL aliquot of silica particle suspension is centrifuged, the supernatant is removed, and 1 mL of the freshly prepared small unilamellar vesicle solution is added and incubated with shaking for 45 min to obtain silica particle supported lipid bilayers. The final mixture is repeatedly centrifuged and washed in dilute PBS to remove excess lipid.

### Example 9

### DNA Bucky Ball Scaffolds

DNA-made Bucky ball traps were constructed by adapting the procedure of He et al. (Nature 452, 198-201, 2008, Hierarchical self-assembly of DNA into symmetric supramolecular polyhedral). Bucky ball architecture is based upon a ‘connector’ DNA assembly constituting the vertices of the polyhedral, and a ‘bar’ DNA assembly constituting the edges of the polyhedral. While the connector sequences are constant, the bar sequences are varied so that either a 100 nm bar size is produced or a 200 nm bar size is produced. Properly folded—using the 100 nm bars—the DNA bucky ball should produce a polyhedral shape with estimated diameter of 500-800 nm. Using 200 nm bars, the diameter of the particle is estimated to be 1-1.4 μm.

The following connector sequences were used for assembly (5′ to 3′):

S′:
(SEQ ID NO: 209)
atactcgctctcgttaccgtgtggttgcatagttttctcgtcac
DaoM:
(SEQ ID NO: 210)
tagcaacctgcctggcaagcctacgatggacacggtaacgcc
L:
(SEQ ID NO: 211)
aggcaccatcgtaggtttcttgccaggcaccatcgtaggtttcttgccaggcaccatcgtaggtttcttgcc

To generate connectors, 1 μM of L sequence, 3 μM of S sequence and 3 μM of DaoM sequence were mixed in 100 μl final volume mixture, in 1×TAE buffer containing 12.5 mM MgCl₂. The reaction mixture was then heated to 95° C., followed by a gradual lowering of temperature to room temperature over 48 hour period.

The following bar sequences were used for assembly (5′ to 3′):

H1:
(SEQ ID NO: 212)
agagcgagtatggagtcagatggcttacttacctatcgcgctat
H2:
(SEQ ID NO: 213)
ttctatacaatttgcgacttatttataccaagtctttattagac
H3:
(SEQ ID NO: 214)
gtgtacagaaactcactgcgtcttaagaatggaacgttgtccat
H4:
(SEQ ID NO: 215)
ttcataagtagtagcacctatgcggcatcgcatttgagtagataggc
H5:
(SEQ ID NO: 216)
acacgtatcggatgtctctgtaggttgtgcagatatagataata
H6:
(SEQ ID NO: 217)
cagttttttagaagcgaagctggaccatgagaagttattcccg
H7:
(SEQ ID NO: 218)
cgatgtcagcgaacacatgtcgatgtatttaaagtgacgagaaa
G1:
(SEQ ID NO: 219)
tttaaatacatcgacatgtgttggtaagtaagccatctgactcc
G2:
(SEQ ID NO: 220)
cgctgacatcgcgggaaataacattgtatagaaatagcgcgata
G3:
(SEQ ID NO: 221)
ttctcatggtccagcttcgcttacttggtataaataagtcgcaa
G4:
(SEQ ID NO: 222)
ctaaaaaactgtattatctatatttctgtacacgtctaataaag
G5:
(SEQ ID NO: 223)
tctgcacaacctacagagacattccattcttaagacgcagtgag
G6:
(SEQ ID NO: 224)
ccgatacgtgtgcctatctactctacttatgaaatggacaacgt
G7:
(SEQ ID NO: 225)
caaatgcgatgccgcataggtgcaaatgcgatgccgcataggtg
G8:
(SEQ ID NO: 226)
ctacttatgaaatggacaacgtccgatacgtgtgcctatctact
G9:
(SEQ ID NO: 227)
tccattcttaagacgcagtgagtctgcacaacctacagagacat
G10:
(SEQ ID NO: 228)
tttctgtacacgtctaataaagctaaaaaactgtattatctata
G11:
(SEQ ID NO: 229)
acttggtataaataagtcgcaattctcatggtccagcttcgctt
G12:
(SEQ ID NO: 230)
attgtatagaaatagcgcgatacgctgacatcgcgggaaataac
G13:
(SEQ ID NO: 231)
ggtaagtaagccatctgactcctttaaatacatcgacatgtgtt

Sequences used to generate a 200 nm bar:

G14:
(SEQ ID NO: 232)
tggcgatacaatgcattccgcaggtaagtaagccatctgactcc
G15:
(SEQ ID NO: 233)
cgctgacatcggttctaatgcc
H8:
(SEQ ID NO: 234)
agagcgagtattgcggaatgcattgtatcgccaggcattagaac

Sequences used to generate a 200 nm bar:

G14:
(SEQ ID NO: 474)
tggcgatacaatgcattccgcaggtaagtaagccatctgactcc
G15:
(SEQ ID NO: 475)
cgctgacatcggttctaatgcc
H8:
(SEQ ID NO: 476)
agagcgagtattgcggaatgcattgtatcgccaggcattagaac

To generate 100 nm bars, 1 μM of each H1-H7 sequences and 1 μM of of each G1-G13 sequences were mixed in 100 μl final volume reaction mixture, in 1×TAE buffer containing 12.5 mM MgCl₂. Reaction mixture was then heated to 95° C., followed by a gradual lowering of temperature to room temperature over 48 hour period.

For the generation of 200 nm bars, 2 μM of each H2-H7 sequences and 1 μM of each G1-G12, and 1 μM of H1, H8, G14 and G15 sequences were mixed in 1000 final volume reaction mixture, in 1×TAE buffer containing 12.5 mM MgCl₂. Reaction mixture was then heated to 95° C., followed by a gradual lowering of temperature to room temperature over 48 hour period.

Following folding, bars and connectors were mixed in a 3:1 bars to connector volume ratio, in 1×TAE buffer containing 12.5 mM MgCl₂.

### Results

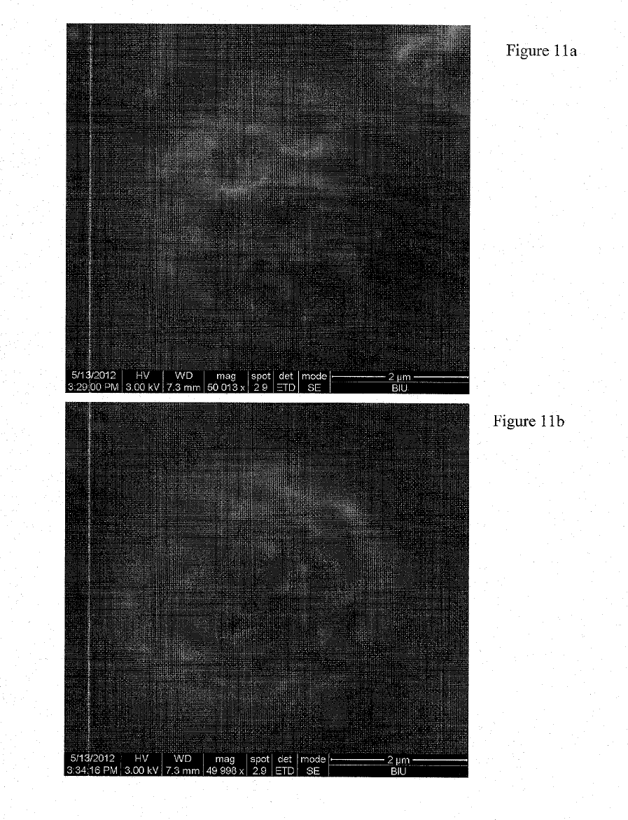

TEM microscopy was conducted for a DNA bucky ball sample, generated with the 100 nm bars (FIG. 12a). The photo shows a spherical shape at the estimated size range of the bucky ball (500-800 nm).

FIG. 12b illustrates the structure and interaction of the DNA sequences that comprise the DNA bucky ball scaffold.

### Example 10

### DNA Spherical Scaffolds

A spherical DNA shape was constructed using DNA-nanotechnology techniques. The spherical shape was generated by connecting two hemispheres each constructed from a mixture of specific DNA sequences.

The sequences used to construct the hemispheres included:

```
  1:
(SEQ ID NO: 235)
ACCCCCATTGTGTTGCGTTACCGTGTGGTTGCATAGTTTTTTATGCATAT
CACTTCT
  2:
(SEQ ID NO: 236)
5′Phos-CTAGCAATATCTATGT
  3:
(SEQ ID NO: 237)
ACTACGTCCCCCCATCGTACGATCAGGCGAGGACTCTGACCAGTCTCCAA
CA
  4:
(SEQ ID NO: 238)
CAACACAATGGGGGTACATAGATATTG
  6:
(SEQ ID NO: 239)
CCACGAGTTCCTTATCCGTAGGGCTATATTGACACTGTTTTTAGAGATGA
AATCTGT
  7:
(SEQ ID NO: 240)
CCACGAGTTCCTTATCCGTAGGGCTATATTGACACTGTTTTTAGAGATGA
AATCTGT
  9:
(SEQ ID NO: 241)
ATAAGGAACTCGTGGACATAGATATTG
10:
(SEQ ID NO: 242)
TGCTTCGTGTGCGACAGAGCTTATCCATTGGCTGGGAGCGGGTCATTGAC
CC
12:
(SEQ ID NO: 243)
GTCGCACACGAAGCAACATAGATATTG
13:
(SEQ ID NO: 244)
GACAACAAACCTGAAAGAGCTTATCCATTGGCTGGGAGGTGTTGCTCCTG
CC
14:
(SEQ ID NO: 245)
GCGGTTCTCGTGAACGCAATATCTATGT
15:
(SEQ ID NO: 246)
5′Phos-CATGACATAGATATTG
16:
(SEQ ID NO: 247)
GACAACAAACCTGAAAGAGCTTATCCATTGGCTGGGAGGTGTTGCTCCTG
CC
17:
(SEQ ID NO: 248)
TTCAGGTTTGTTGTCCAATATCTATGT
19:
(SEQ ID NO: 249)
TGCTGCAGGCGCAAGCGTACGATCAGGCGAGGACTCTCCCAACACATAAA
GA
20:
(SEQ ID NO: 250)
5′Phos-CTAGACATAGATATTG
21:
(SEQ ID NO: 251)
CTTGCGCCTGCAGCACAATATCTATGT
22:
(SEQ ID NO: 252)
TATTTCGGCGTTAACCCGTAGGGCTATATTGACACTGTTTTTTGTCAGGG
CGTAGCA
23:
(SEQ ID NO: 253)
ATCGTTGTATGCTAGCCGTAGGGCTATATTGACACTGTTTTTATAGACTA
GAACCTT
24:
(SEQ ID NO: 254)
AGCCATGATTACATTCGTTACCGTGTGGTTGCATAGTTTTTTGTTACGCG
CAAATTT
25:
(SEQ ID NO: 255)
AATGTAATCATGGCTCAATATCTATGT
27:
(SEQ ID NO: 256)
TCACGCGAAGAGGCTAGAGCTTATCCATTGGCTGGGACCGACATGCTAAA
AG
28:
(SEQ ID NO: 257)
AGCCTCTTCGCGTGACAATATCTATGT
30:
(SEQ ID NO: 258)
CTGCGACGCTGAGGCCGTACGATCAGGCGAGGACTCTTAACAGGATGCAA
AT
31:
(SEQ ID NO: 259)
5′Phos-CTAGACATAGATATTG
32:
(SEQ ID NO: 260)
GCCTCAGCGTCGCAGCAATATCTATGT
33:
(SEQ ID NO: 261)
ATCGGCATTACGCTTCCGTAGGGCTATATTGACACTGTTTTTATCGGCAT
TACGCTT
34:
(SEQ ID NO: 262)
AAGCGTAATGCCGATCCGTAGGGCTATATTGACACTGTTTTTTATAACGG
GACAACG
35:
(SEQ ID NO: 263)
TCGGGGAGTACCTCTCGTTACCGTGTGGTTGCATAGTTTTTTGTTTTTAT
TATAGAA
36:
(SEQ ID NO: 264)
AGAGGTACTCCCCGACAATATCTATGT
38:
(SEQ ID NO: 265)
GCGGCTTGATTATATCGTACGATCAGGCGAGGACTCTCCGACGGGCACAT
AC
40:
(SEQ ID NO: 266)
ATATAATCAAGCCGCCAATATCTATGT
41:
(SEQ ID NO: 267)
ATTCCGCATGAGCGAAGAGCTTATCCATTGGCTGGGATTTGGAGCAGTCC
CA
42:
(SEQ ID NO: 268)
TCGCTCATGCGGAATCAATATCTATGT
44:
(SEQ ID NO: 269)
CTAGCATACAACGATCCGTAGGGCTATATTGACACTGTTTTTGGTCGCAT
GCATCGT
45:
(SEQ ID NO: 270)
GTTAACGCCGAAATACCGTAGGGCTATATTGACACTGTTTTTTGTTCAGC
TCCTCCG
46:
(SEQ ID NO: 271)
CAAGTCTGTCGATAACGTTACCGTGTGGTTGCATAGTTTTTTTCTGTAGA
GCGGAAT
47:
(SEQ ID NO: 272)
TTATCGACAGACTTGCAATATCTATGT
49:
(SEQ ID NO: 273)
AGTGGCGCTTGCTTACGTACGATCAGGCGAGGACTCTTCTGGCCATCGAT
AA
51:
(SEQ ID NO: 274)
TAAGCAAGCGCCACTCAATATCTATGT
52:
(SEQ ID NO: 275)
AGACGCCGTGTTGACAGAGCTTATCCATTGGCTGGGAAAAGGCGACAGCA
TT
53:
(SEQ ID NO: 276)
GTCAACACGGCGTCTCAATATCTATGT
55:
(SEQ ID NO: 277)
GAGTGAAGTAAGAGCCCGTAGGGCTATATTGACACTGTTTTTTGGCGCAC
ATAACTA
56:
(SEQ ID NO: 278)
GCTCTTACTTCACTCCCGTAGGGCTATATTGACACTGTTTTTTGTTCATT
TTGGAAT
60:
(SEQ ID NO: 279)
AAAAAACTATGCAACCTGCCTGGCAAGCCTACGATGGGGATAAGCTCT
61:
(SEQ ID NO: 280)
TCCCAGCCAATTGCCTGGCAAGCCTACGATGGTAGCCCTACGG
62:
(SEQ ID NO: 281)
AAAAACAGTGTCAATATGCCTGGCAAGCCTACGATGGCTGATCGTACG
63:
(SEQ ID NO: 282)
AGAGTCCTCGCTGCCTGGCAAGCCTACGATGGACACGGTAACG
64:
(SEQ ID NO: 283)
AGGCACCATCGTAGGTTTCTTGCCAGGCACCATCGTAGGTTTCTTGCCAG
GCACCATCGTAGGTTTCTTGCCAGGCACCATCGTAGGTTTCTTGCC
```

Each hemisphere includes 8 bars and 5 plus-shaped connectors, one polar connector and 4 equatorial connectors, linking the bars to one another. While connectors include 8 custom made synthetized DNA sequences, bars are DNA fragments cut from Lambda phage DNA by restriction enzymes. The size of the DNA sphere is a direct result of the size of the bar fragments of Lambda DNA used, which is dependent on the restriction enzymes used. The 4 equatorial connectors of one hemisphere are designed to attach to their respective connector partners at the other hemisphere. Connectors are termed herein as Polar, South America, South Atlantic, South Asia, South Pacific, North America, North Atlantic, North Asia, North Pacific. The 8 equatorial connectors of one hemisphere are designed to attach to their respective connector partner on the other hemisphere, so that connector North America attaches to connector South Asia, connector North Asia attaches to connector South America, connector North Pacific attached to connector South Pacific and connector North Atlantic attaches to connector South Pacific.

The following sequences were used to generate the connectors:
Polar connector: 1, 4, 7, 10, 60, 61, 62, 63, 64.
North America connector: 13, 16, 19, 22, 60, 61, 62, 63, 64.
South America connector: 13, 16, 19, 23, 60, 61, 62, 63, 64.
North Atlantic connector: 24, 27, 30, 33, 60, 61, 62, 63, 64.
South Atlantic connector: 24, 27, 30, 33, 60, 61, 62, 63, 64.
North Asia connector: 35, 38, 41, 44, 60, 61, 62, 63, 64.
South Asia connector: 35, 38, 41, 45, 60, 61, 62, 63, 64.
North Pacific connector: 46, 49, 52, 55, 60, 61, 62, 63, 64.
South Pacific connector: 46, 49, 52, 56, 60, 61, 62, 63, 64.

To generate connectors, 1 μM of each connector sequence were mixed in 100 μl final volume mixture, in 1×TAE buffer containing 12.5 mM MgCl₂. Reaction mixture was then heated to 95° C., followed by a gradual lowering of temperature to room temperature over 48 hour period.

For initial proof of concept, a 4716 bp Lambda DNA fragment, was used as bar. 100 μg Lambda phage DNA (New England BioLabs) was cut using 100 U of NheI (New England BioLabs) and 150 U of PciI (New England BioLabs) with 5 μg BSA in 100 μl final volume in buffer 2 (New England BioLabs). Reaction was incubated at 37° C. for 1 hour. Following gel electrophoresis in 1% agarose, the DNA fragment was cut from the gel and extracted using QIAquick gel extraction kit (Qiagen).

Specific, custom made, synthetic DNA sequences were attached to the bars to serve as adaptors so that bars could be linked to the connectors. adaptors were generated using the following sequences: Sequences used to generate adaptors for attaching bars to the Polar connectors:
PolAm: 2, 3
PolAt: 2, 6
PolAs: 2, 9
PolPa: 2, 12

Sequences used to generate adaptors for attaching bars to the America connectors:
AmPol: 14, 15
AmAt: 17, 15
AmPa: 2, 21

Sequences used to generate adaptors for attaching bars to the Atlantic connectors:
AtPol: 25, 15
AtAs: 28, 15
AtAm: 2, 32

Sequences used to generate adaptors for attaching bars to the Asia connectors:
AsPol: 36, 15
AsAt: 2, 40
AsPa: 42, 15

Sequences used to generate adaptors for attaching bars to the Pacific connectors:
PaPol: 47, 15
PaAs: 2, 51
PaAm: 53, 15

Adapters were generated at a 1×TAE buffer 25 μl final volume, using final concentration of 100 nM from the phosphorylated sequences and 200 nM final concentration from the complementary, unphosphorylated sequence. Mixtures were kept at room temperature for 5 minutes.

113 nmole bars are mixed with 113 nmole adaptors in a ligation reaction mixture containing 1000 U of T4 ligase (New England BioLabs) at 1×T4 ligase biffer (New England BioLabs). Reaction were incubated at room temperature for 10 minutes.

Following gel electrophoresis in 1% agarose, the DNA fragments were cut from the gel and extracted using QIAquick gel extraction kit (Qiagen).

Ligation reactions contain bars with the following adaptors:
Bar1: PolAm, AmPol.
Bar2: PolAt, AtPol.
Bar3: PolAs, AsPol.
Bar4: PolPa, PaPol.
Bar5: AmAt, AtAm.
Bar6: AmPa, PaAm.
Bar7: AtAs, AsAt.
Bar8: AsPa, PaAs.

Hemispheres were generated using a final concentration of 1.8 nM of each of the bars, and a 1.8 μl of each of the connectors, in 1×TAE buffer, containing 12.5 mM MgCl₂. Reaction mixtures were incubated at room temperature for 5 hours.

The reaction mixture for generating the North Hemisphere included:

Polar connector, North America connector, North Pacific connector, North Asia connector, North Atlantic connector, Bar1, Bar2, Bar3, Bar4, Bar5, Bar6, Bar7, Bar8. The reaction mixture for generating the South Hemisphere included:

Polar connector, South America connector, South Pacific connector, South Asia connector, South Atlantic connector, Bar1, Bar2, Bar3, Bar4, Bar5, Bar6, Bar7, Bar8. Equal volumes of North Hemispher and South Hemisphere were mixed to produce whole spheres. Mixture was incubated at room temperature for 1 hour.

The plus shaped connectors are designed to have a curvature, facilitating the sphere shape. In addition, each connector has two DNA sequences perturbing outside of the sphere, and two DNA sequences perturbing inside the sphere. The outside facing sequences are used to attach trap-directing moieties to the spheres, such as tissue specific antibodies. The inside facing sequences are used to attach active agents to the sphere. 4 DNA sequences are designed to attach to these inside perturbing sequences, and through self-aggregation create a DNA mesh, thereby increasing the number of active agents binding sites:

(SEQ ID NO: 284)
MESH1: CGCTATACGTGTTCACCGCTTGCTAGCAGT
(SEQ ID NO: 285)
MESH2: CGCTATAGCAAGCGGACTCTGGCCTTCGAT
(SEQ ID NO: 286)
MESH3: CGCTATAGCCAGAGTGGAAGGCGAGGATCA
(SEQ ID NO: 287)
MESH4: CGCTATACGCCTTCCTGAACACGGTTACAG

The end sequences of the sheet are capped by DNA sequences that are modified to have an amino group at their 5′ end to facilitate easy binding of active agents, either through biotinylation of the amino group or by direct linking of the active agent to the amino group:

(SEQ ID NO: 288)
LOL1: 5′-AmMC6-TTTCTGTAAC
(SEQ ID NO: 289)
LOL2: 5′-AmMC6-TTTACTGCTA
(SEQ ID NO: 290)
LOL3: 5′-AmMC6-TTTATCGAAG
(SEQ ID NO: 291)
LOL4: 5′-AmMC6-TTTTGATCCT

Initially the mesh sequences MESH1-4 are added at equimolar ratios, followed by incubation at room temperature for 1 hour. Then, the 5′-amino-modified sequences are added at equimolar ratios, followed by incubation at room temperature for 30 minutes.

### Example 11

### DNA-Origami Scaffold Cube

A three-dimensional DNA cube was constructed using DNA origami techniques. The cube shape is comprised of four, three-armed corners, designed to connect to each other. The shapes are comprised of M13 phage single-strand DNA genome (Taxonomy ID: 10870) sequence, and 191 custom-made single-strand DNA staples. The DNA staples used to construct the cube are listed:

```
(SEQ ID NO: 292)
    2 CCAACGTCAATTCCAGTTCCCTTAAGCA
(SEQ ID NO: 293)
    5 GGTTCCGAAATCGGCAAAATTGGAACAGGGATACCTCAGAGCCACCACC
(SEQ ID NO: 294)
    6 GTCCACGCTGGTGTTAGCGTA
(SEQ ID NO: 295)
    7 GGCTGGCCCTCTTTTCAGCGC
(SEQ ID NO: 296)
    8 CTGATTGCCCTTCACCGCCGAAAATCATTCCACCAGTACAAACTACAAC
(SEQ ID NO: 297)
    9 GUGCGCCAGGGTACTTTCAAC
(SEQ ID NO: 298)
  10 GGGAGTCGGGTGAGCTATACG
(SEQ ID NO: 299)
  11 CATTAATGAATCGGCCAACCCAGTGATGTATGGTCCAGACGTTAGTAAA
(SEQ ID NO: 300
  12 CGCTCACTGCCCAATCTCCAA
(SEQ ID NO: 301)
  13 GCCTGGGGTGCCTAATGAGAAACCTGTAATAATAAGGAACAACTAAAGG
(SEQ ID NO: 302)
  14 CTCACAATTCCAACAGCTTGA
(SEQ ID NO: 303)
  15 AGCTGTTTCCAGGATCCAACG
(SEQ ID NO: 304)
  16 TCGTAATCATGGTCATAGCCGGAAGCCTTTCGATTAATTGTATCGGTT
(SEQ ID NO: 305)
  17 GCCTGCAGGTCGCTTGCAGGG
(SEQ ID NO: 306)
  18 ACGATTAAGTTTCGCTAAGGT
(SEQ ID NO: 307)
  19 CCCAGTCACGACGTTGTAACCGGGTACGATATAACAACAACCATCGCCC
(SEQ ID NO: 308)
  20 GGGGATGTGCTGCGGCTACAG
(SEQ ID NO: 309)
  21 GGCCTCTGGGTAAGCATCGGTCGTCACCCTCAGCAG
(SEQ ID NO: 310)
  22 GGAAGGGCGAGCGCATCATGTGAGTGTATAAG
(SEQ ID NO: 311)
  23 ATTCAGGCTGGCCAGTTGCCAGCTTGTAAAC
(SEQ ID NO: 312)
  24 ACCAGGCAAAGCAGTATCTTCGCGTAAAATTC
(SEQ ID NO: 313)
  25 TTCCGGCACCGCTTCTCGCACTCTAGGAACTTAAATC
(SEQ ID NO: 314)
  26 GGATTGACCGTACTCCATGTT
(SEQ ID NO: 315)
  27 CACTCGGATTAAGCCCCAAAA
(SEQ ID NO: 316)
  28 CAACCCGGTTGGTGTGTG
(SEQ ID NO: 317)
  29 TCCTGTATGAGGGGGAAA
(SEQ ID NO: 318)
  31 TGATAATCAGAACTCCGTGGGAACAAACGGC
(SEQ ID NO: 319)
  32 ACAAGCATGTCGGTCAAACCAGAAATTACCTTATGC
(SEQ ID NO: 320)
  33 CAAATAATGAACGAACTGACAGGCTTGC
(SEQ ID NO: 321)
  34 AATTTCATCAACATTAAGTAACCGTTGTATCAACGGGTAAAA
(SEQ ID NO: 322)
  35 GTTAATACTGGAGCGACAGATAAGCTGCTCATTCAAAAGAATTATTTCA
(SEQ ID NO: 323)
  36 GCATTAAATCAGGTCCAGGCGATTACCCAAATCAAAATA
(SEQ ID NO: 324)
  38 GCTATTTTTGGCCATCAAAAATAAGGCCTCACCCAGCGAAACGAA
(SEQ ID NO: 325)
  40 TAATGTGTAGGTATCiTACCCCGGT
(SEQ ID NO: 326)
  41 CAAAAGGCATATATTAGCATTTTGGGGCATAAAACGAACT
(SEQ ID NO: 327)
  42 GACAGTGATAAAACATACAGG
(SEQ ID NO: 328)
  43 TATGATAAAGCCTTTAGCAAAA
(SEQ ID NO: 329)
  44 ACCTGAGAGTTTTTGTTCTGGCCT
(SEQ ID NO: 330)
  45 AACCCTGTGAGAATAAAACTGGAAGATCGAGTAA
(SEQ ID NO: 331)
  46 ACGCAAGCAAATCAAGAATCGTTTA
(SEQ ID NO: 332)
  47 GTACCAAAAACATTATGACATTAATGAAAG
(SEQ ID NO: 333)
  48 TCAATTCTACTATAAATTGGAAC
(SEQ ID NO: 334)
  49 TAGTTTAAATGCAATGCCTGAG
(SEQ ID NO: 335)
  50 CAAGGCGTGAATACAACTTTCGGAGATTGCATCTCGCAACTGTTG
(SEQ ID NO: 336)
  51 TTAAGCCGTAACAGAACGGTCAAGCGCACGACGACGCCATTCGCC
(SEQ ID NO: 337)
  52 AAGTACTTTTGCGGGAGTTCA
(SEQ ID NO: 338)
  53 CATAAAGATATTCCATAGGCTTTGACCGGAAGATGGTGCCGGAA
(SEQ ID NO: 339)
  54 CTATATTTTCATAACATCCGAGAAACTCATAAGGATA
(SEQ ID NO: 340)
  55 ATTAGATACATTGAAAAGGTGGCA
(SEQ ID NO: 341)
  56 AACCCATATAATGTTTTTACCAGACGACG
(SEQ ID NO: 342)
  57 TCCCAATTCTAACCTGTTTAG
(SEQ ID NO: 343)
  58 GGTGTCTGGAAGAGGTAGAAAAA
(SEQ ID NO: 344)
  59 ATTGAGTAGATTTAGTTTGACC
(SEQ ID NO: 345)
  60 ACACAGTTGAT
(SEQ ID NO: 346)
  61 TAATTGCTGAATCAACTAAAGTAC
(SEQ ID NO: 347)
  62 TGGCTTAGAGCT
(SEQ ID NO: 348)
  63 TTTCTTTAATAACCAGATAAACAGTTCAG
(SEQ ID NO: 349)
  64 TGATAAGAGCA
(SEQ ID NO: 350)
  65 GGTCAGGATTAGGAGGCTTTCAT
(SEQ ID NO: 351)
  66 CGTGCTCCTTT
(SEQ ID NO: 352)
  67 ATATCGCGTTTTCAAACTCCAACA
(SEQ ID NO: 353)
  68 CTTCAATTAAGAGAGCAAAGCGGATTGCA
(SEQ ID NO: 354)
  71 AAAACGAGAATGACCATAAAGTCAGAGAAGCCCGAAAGACTTCAA
(SEQ ID NO: 355)
  73 GTCGGGTAATAGTAAAAATAGCGAAGAGTACTTGCGGA
(SEQ ID NO: 356)
  74 ATAGCGTCCAATACTGCGGAATGCTTCCGGAAGAATTCGAG
(SEQ ID NO: 357)
  75 TTTTGCCAGAGGATAAATATT
(SEQ ID NO: 358)
  76 ATAAAAACCAAATGTTTAGACTGG
(SEQ ID NO: 359)
  77 GCAACACTATTGCAAAAGAAG
(SEQ ID NO: 360)
  78 AACACGAGGCAATACCACATTCAATTATTACTTTC
(SEQ ID NO: 361)
  79 GTTAAATATGATAATGCTGTAGCTGTCA
(SEQ ID NO: 362)
  80 CATAACGCCAAAAGGAATTCCTC
(SEQ ID NO: 363)
  81 TTGAGATTTAGGATAGTAAGA
(SEQ ID NO: 364)
  82 AACGGAACAACACTAATGCACIATA
(SEQ ID NO: 365)
  83 GTTGGGAAGAAGATTCATCAG
(SEQ ID NO: 366)
  84 ATCTTATACCTTTAATCATTGTGACGAGTAGATAG
(SEQ ID NO: 367)
  85 TTAGCGAGCTTCGCAAATGGTCAATGCG
(SEQ ID NO: 368)
  86 GATTTTAAGAACTGGCTCATACG
(SEQ ID NO: 369)
  87 TTTAATTTCAACAGTCAGGAC
(SEQ ID NO: 370)
  88 CCTGACAATAAATATTTTTAG
(SEQ ID NO: 371)
  89 GTAATCTTGACAAGAACCGGCTAAATCGGTT
(SEQ ID NO: 372)
  90 ACTTAGCCGGGCTTGAGATGG
(SEQ ID NO: 373)
  91 GAGCGACCTGATGGGATTTACGCCAGCTGGCGAAAG
(SEQ ID NO: 374)
  92 AGACATTGCCGTTCTAGCTGATAACCTGTAACCTCAGAG
(SEQ ID NO: 375)
  93 CCGGCGCAGACAATCATAAAGATT
(SEQ ID NO: 376)
  94 TCGTTCATGAGGAAGTTAAAGACACGCCAGGGTTTT
(SEQ ID NO: 377)
  95 AATTAGATGGTCGGTGCG
(SEQ ID NO: 378)
  96 CAACACTACGAAGGCACCAACCTAATTATACGTAC
(SEQ ID NO: 379)
  97 AAAACACTCATCTGGCTGAGATCTACCCGGAGAGGGTAGCTA
(SEQ ID NO: 380)
  98 AGGCTTTGAGGACTAAGTAGCAACAAGGCGGCCAGTGCCAAGCTTGCAT
(SEQ ID NO: 381)
  99 TACGTAATGCAGTACAAGAAAGAGAAACAAGCCATCAA
(SEQ ID NO: 382)
100 AGAGGCAAAAGAATACACT
(SEQ ID NO: 383)
101 CGTCCATTAATCGCCTGGAACCGGTAATCGAGGCCGGA
(SEQ ID NO: 384)
102 AGTTAAAGGCCGCTTTGCTGAGGACTCTAGTGTGTGAAATTGTTATCCG
(SEQ ID NO: 385)
103 GGAAACGAGGAGACTTTAAAT
(SEQ ID NO: 386)
104 ACGCATAACCCGAGCTCGAAT
(SEQ ID NO: 387)
105 TACCGATAGTTGCGCCTTCTTAACACAACAACTCACATTAATTGCGTTG
(SEQ ID NO: 388)
106 AATGACAATGTTCGGTCTGCG
(SEQ ID NO: 389)
107 ATCAGCTTGATAAAGTGTAAA
(SEQ ID NO: 390)
108 AAAAAAGGCTCCAAAACGTTGAAGCTTTCCGAGAGGCGGTTTGCGTATT
(SEQ ID NO: 391)
109 AATTGCGAATCGTGCCAGCTG
(SEQ ID NO: 392)
110 AGTTTCAGCGGAGTGACTAAACAGGTTTTTGAGAGAGTTGCAGCAAGCG
(SEQ ID NO: 393)
111 AGATTTTTCAGGAGCCTGGTG
(SEQ ID NO: 394)
112 TGAATTTTCGACGGGCAACAG
(SEQ ID NO: 395)
113 ACGATCTAAAGTTTTGCCTCATATTGCCCCTAAATCAAAAGAATAGCCC
(SEQ ID NO: 396)
114 AGCTCGTCTTGATTTTGGAAT
(SEQ ID NO: 397)
115 GCCTGTAGCCTGTTTGATGGT
(SEQ ID NO: 398)
116 ACCGTAACACTGAGTTCAATAGGAGTGTTGAGGGCGAAAAA
(SEQ ID NO: 400)
117 CTCATTTTCAAGAGTCCACTA
(SEQ ID NO: 401)
118 TTTTTTTTCTCAGAACGT
(SEQ ID NO: 402)
119 AACCGCCACGCAAGCCTCGTCACAGAC
(SEQ ID NO: 403)
120 CATTGAATCATCAGGTCTCGCAAGAAAGTCGGGACGAGT
(SEQ ID NO: 404)
121 CATATTCTGTGTAAACCAGAGTCAAAAAGAAG
(SEQ ID NO: 405)
122 AGCGACGATCTCGGACCCGCATTACCCTGACTATTATATCAAAACCCCTCAAATC
(SEQ ID NO: 406)
123 CATTGAATCATCAGGTCTCACGGCAAGATAACTGCGGAT
(SEQ ID NO: 407)
124 CCTTCCCACCGCCTAGCGAGTTCAAAAAGAAG
(SEQ ID NO: 408)
125 GTCTAGGAAGTCTTCTACCTGTTACCCTGACTATTATATCAAAACCCCTCAAATC
(SEQ ID NO: 409)
126 CATTGAATCATCAGGTCTTACTATGGGAGACGTTTCTCC
(SEQ ID NO: 410)
127 TATAGGGCCGGCTTCATAGAGTCAAAAAGAAG
(SEQ ID NO: 411)
128 CCTGACTGAAAGTTACTCTTTTTACCCTGACTATTATATCAAAACCCCTCAAATC
(SEQ ID NO: 412)
129 CATTGAATCATCAGGTCTTTTCTTGTGGACTCTAGACCT
(SEQ ID NO: 413)
130 ACTTTAGACGCCCCACCTTTATCAAAAAGAAG
(SEQ ID NO: 414)
131 CAACGCAGCCACGAAGCACTATTACCCTGACTATTATATCAAAACCCCTCAAATC
(SEQ ID NO: 415)
132 CATTGAATCATCAGGTCTTATCCCTCGGTCAGAGAAACT
(SEQ ID NO: 416)
133 GATATGGTTCTATTACGCTCATCAAAAAGAAG
(SEQ ID NO: 417)
134 GCGTCTACGGTAAACATAAGCTTACCCTGACTATTATATCAAAACCCCTCAAATC
(SEQ ID NO: 418)
135 CATTGAATCATCAGGTCTTCATGCTATCACACCATGTCG
(SEQ ID NO: 419)
136 GTCGGGCAGAAATATGGATCGTCAAAAAGAAG
(SEQ ID NO: 420)
137 ACTTCGTGGTGACTGACGGTATTACCCTGACTATTATATCAAAACCCCTCAAATC
(SEQ ID NO: 421)
138 CATTGAATCATCAGGTCTTGAGGATGTAGTGTTGCTCAC
(SEQ ID NO: 422)
139 CTCCGTGATTCCTATAGCAGGTCAAAAAGAAG
(SEQ ID NO: 423)
140 AGCGAAAGCAAGGTGACTTGTTTACCCTGACTATTATATCAAAACCCCTCAAATC
(SEQ ID NO: 424)
141 CATTGAATCATCAGGTCTATCTCCGCCGAAAAGTTCAGT
(SEQ ID NO: 425)
142 ACACCCTGGGCAAGTGCCAGCTCAAAAAGAAG
(SEQ ID NO: 426)
143 GCTCGAATTCGAACCATTCCITTACCCTGACTATTATATCAAAACCCCTCAAATC
(SEQ ID NO: 427)
144 AGCTCATTTGTCGCTGGGGTGCTGGCGTCC
(SEQ ID NO: 428)
145 GTTACGTCTATCTGGGGAGGCTTTAACCAACAGCCAGCT
(SEQ ID NO: 429)
146 GGTAGTCGCGAACACGCGAAGTTTTTGAGACCTTCATCAAGA
(SEQ ID NO: 430)
147 AGCTCATTTGTATTTGTATTTGAGGAAAGGCCCTGC
(SEQ ID NO: 431)
148 ATTATGGGTGTACGGACGCTATTTAACCAACAGCCAGCT
(SEQ ID NO: 432)
149 GCTCAACCTGCTTTTCTGCTGTTTTTGAGACCTTCATCAAGA
(SEQ ID NO: 433)
150 AGCTCATTTGCAGGGCCTTTCCTCAAATAC
(SEQ ID NO: 434)
151 TAGCGTCCGTACACCCATAATTTTAACCAACAGCCAGCT
(SEQ ID NO: 435)
152 CAGCAGAAAAGCAGGTTGAGCTTTTTGAGACCTTCATCAAGA
(SEQ ID NO: 436)
153 AGCTCATTTGGACGCCAGCACCCCAGCGAC
(SEQ ID NO: 437)
154 GCCTCCCCAGATAGACGTAACTTTAACCAACAGCCAGCT
(SEQ ID NO: 438)
155 CTTCGCGTGITCGCGACTACCTTTTTGAGACCTTCATCAAGA
(SEQ ID NO: 439)
156 AGCTCATTTGTTGACGGCGTTCTCACAGAA
(SEQ ID NO: 440)
157 GATATTTCTCATTTTCTTCACTTTAACCAACAGCCAGCT
(SEQ ID NO: 441)
158 CTACATGTACTGATAAGTGCTTTTTTGAGACCTTCATCAAGA
(SEQ ID NO: 442)
159 AGCTCATTTACTGCCTTTACAATATCAATG
(SEQ ID NO: 443)
160 GATATTTCTCATTTTCTTCACTTTAACCAACAGCCAGCT
(SEQ ID NO: 444)
161 CCTCAAATGCTCGCACTTTTATTTTTGAGACCTTCATCAAGA
(SEQ ID NO: 445)
162 AGCTCATTTCATTGATATTGTAAAGGCAGT
(SEQ ID NO: 446)
163 GTGAAGAAAATGAGAAATATCTTTAACCAACAGCCAGCT
(SEQ ID NO: 447)
164 TAAAAGTGCGAGCATTTGAGGTTTTTGAGACCTTCATCAAGA
(SEQ ID NO: 448)
165 AGCTCATTTTTCTGTGAGAACGCCGTCAAC
(SEQ ID NO: 449)
166 ATAATTAACTGGGTACCTCTATTTAACCAACAGCCAGCT
(SEQ ID NO: 450)
167 AGCACTTATCAGTACATGTAGTTTTTGAGACCTTCATCAAGA
(SEQ ID NO: 451)
168 CCGTCTATCAATCCGCAGTTATCTTGCCGTG
(SEQ ID NO: 452)
169 ACTCGCTAGGCGGTGGGAAGGTTAAAGAACGTGGACT
(SEQ ID NO: 453)
170 CAGGTAGAAGACTTCCTAGACGAGATAGGGTTGAACCCATCGCCACCCTCAG
(SEQ ID NO: 454)
171 CCGTCTATCACGACATGGTGTGATAGCATGA
(SEQ ID NO: 455)
172 CGATCCATATTTCTGCCCGACTTAAAGAACGTGGACT
(SEQ ID NO: 456)
173 TACCGTCAGTCACCACGAAGTCGAGATAGGGTTGAACCCATCGCCACCCTCAG
(SEQ ID NO: 457)
174 CCGTCTATCAAGGTCTAGAGTCCACAAGAAA
(SEQ ID NO: 458)
175 TAAAGGTGGGGCGTCTAAAGTTTAAAGAACGTGGACT
(SEQ ID NO: 459)
176 TAGTGCTTCGTGGCTGCGTTGGAGATAGGGTTGAACCCATCGCCACCCTCAG
(SEQ ID NO: 460)
177 CCGTCTATCAACTGAACTTTTCGGCGGAGAT
(SEQ ID NO: 461)
178 GCTGGCACTTGCCCAGGGTGTTTAAAGAACGTGGACT
(SEQ ID NO: 462)
179 AGGAATGGTTCGAATTCGAGCGAGATAGGGTTGAACCCATCGCCACCCTCAG
(SEQ ID NO: 463)
180 CCGTCTATCAACTCGTCCCGACTTTCTTGCG
(SEQ ID NO: 464)
181 CTCTGGTTTACACAGAATATGTTAAAGAACGTGGACT
(SEQ ID NO: 465)
182 TGCGGGTCCGAGATCGTCGCTGAGATAGGGTTGAACCCATCGCCACCCTCAG
(SEQ ID NO: 466)
183 CCGTCTATCAAGTTTCTCTGACCGAGGGATA
(SEQ ID NO: 467)
184 TGAGCGTAATAGAACCATATCTTAAAGAACGTGGACT
(SEQ ID NO: 468)
185 GCTTATGTTTACCGTAGACGCGAGATAGGGTTGAACCCATCGCCACCCTCAG
(SEQ ID NO: 469)
186 CCGTCTATCAGGAGAAACGTCTCCCATAGTA
(SEQ ID NO: 470)
187 CTCTATGAAGCCGGCCCTATATTAAAGAACGTGGACT
(SEQ ID NO: 471)
188 AAAGAGTAACTTTCAGTCAGGGAGATAGGGTTGAACCCATCGCCACCCTCAG
(SEQ ID NO: 472)
189 CCGTCTATCAGTGAGCAACACTACATCCTCA
(SEQ ID NO: 473)
190 CCTGCTATAGGAATCACGGAGTTAAAGAACGTGGACT
(SEQ ID NO: 399)
191 ACAAGTCACCTTGCTTTCGCTGAGATAGGGTTGAACCCATCGCCACCCTCAG
```

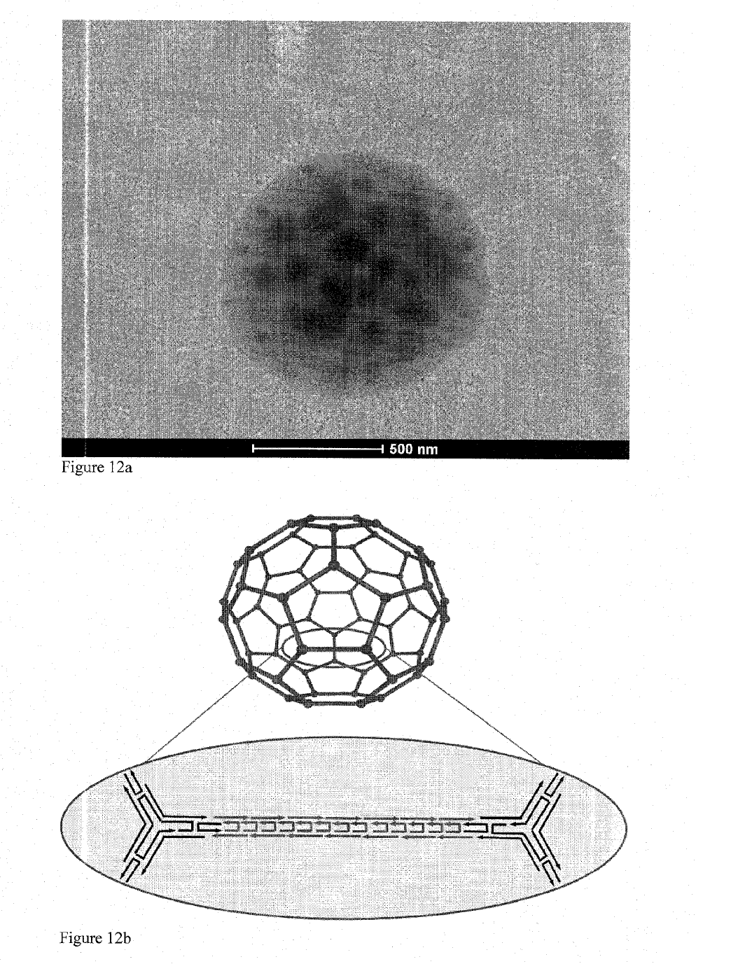

The eight cube corners (FIG. 13) were created by mixing sequences according to Table 2 below.

TABLE 2

| | General | |
|---|---|---|
| Corner | Staples | Corner Specific Staples |
| A | 2-119 | 120, 121, 122, 144, 145, 146, 168, 169, 170 |
| B | 2-119 | 123, 124, 125, 147, 148, 149, 171, 172, 173 |
| C | 2-119 | 126, 127, 128, 150, 151, 152, 174, 175, 176 |
| D | 2-119 | 129, 130, 131, 153, 154, 155, 177, 178, 179 |
| A′ | 2-119 | 132, 133, 134, 156, 157, 158, 180, 181, 182 |
| B′ | 2-119 | 135, 136, 137, 159, 160, 161, 183, 184, 185 |
| C′ | 2-119 | 138, 139, 140, 162, 163, 164, 186, 187, 188 |
| D′ | 2-119 | 141, 142, 143, 165, 166, 167, 189, 190, 191 |

Sequences were mixed to 150 μl final volume, in 1×TAE buffer containing 12.5 mM MgCl₂, with a molar ratio of 4:1 staple to M13 genome. Reaction mixture was then heated to 95° C., followed by a gradual lowering of temperature to room temperature over 48 hour period. Equal volume of each of the reaction mixtures were mixed together to produce a final cube.

### Example 12

### Host and Virus Specific Peptide Active Moieties

Virions express on their exterior surface proteins that originate either from the viral genome or from the host cell. Peptide active moieties were sought for the selective binding of Influenza virus and other virions (“Cellular proteins in influenza virus particles” [Shaw et al., PLoS Pathog. 2008 Jun. 6; 4(6):e1000085]). In silico peptide libraries were generated for the specific binding to host-specific and virus-specific proteins.

### Methods

In silico screening of virtual peptides was performed using the Pepticom software package to select for peptides which specifically and effectively bind solved 3D structures of three target proteins which are presented on exterior surface of Influenza virions: Human CD81 (PDB TD: 1G8Q), Human Annexin2 (PDB ID: 1W7B) and Influenza Neuraminidase (PDB ID: 2BAT). The Tetraspanin and Annexin are examples of protein families that include several host-specific proteins present on Influenza virions and are exemplified by Human CD81 and Human Annxin2 represents respectively.

### Results

A virtual library of selective peptides was generated for each of the protein targets.

A sample of peptides that are predicted to have a high affinity to target proteins and their properties are described in Table 3.

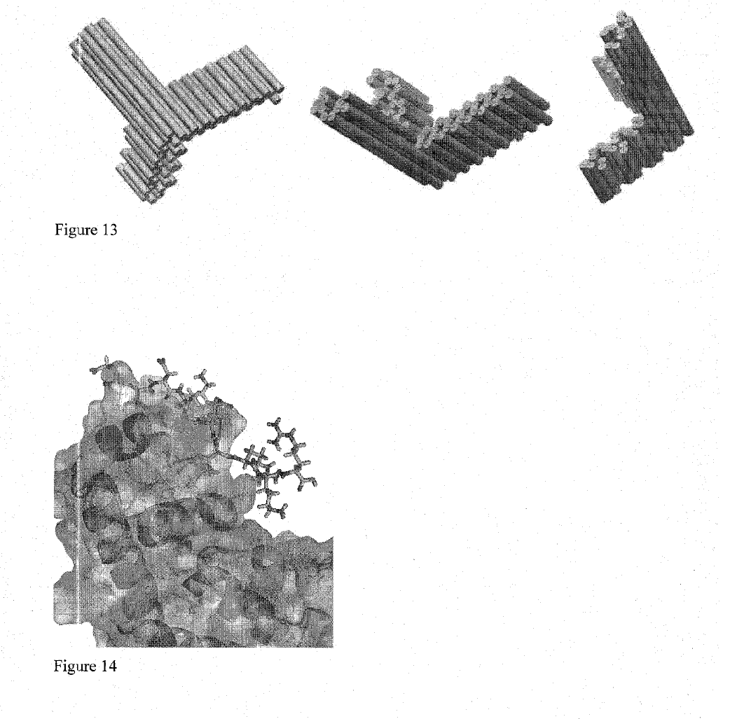

The interaction of peptide No. 3 (SEQ ID NO. 480) with its target protein Human CD81 is further illustrated in FIG. 14.

TABLE 3

| Peptide | | | Predicted affinity | | |
|---|---|---|---|---|---|
| No. | FASTA Sequence | Length | (kcal/mol) | Target protein | SEQ ID NO |
| 1 | IGEVWSKMEKGG | 12 | −26.9 | Human CD81 | 477 |
| 2 | GMSTRSKTKP | 10 | −26.9 | Human CD81 | 478 |
| 3 | ESDKPETRKR | 10 | −16.9 | Human CD81 | 479 |
| 4 | MTMGHKYRMKGEPLE | 15 | −30.3 | Human Annexin2 | 480 |
| 5 | KMKKYSEYW | 9 | −20.2 | Human Annexin2 | 481 |
| 6 | HEGPHESTE | 9 | −23.1 | Human Annexin2 | 482 |
| 7 | SSGEMRW | 7 | −18.8 | Influenza Neuraminidase | 483 |
| 8 | STEEEEW | 7 | −34.4 | Influenza Neuraminidase | 484 |
| 9 | SFSEMTW | 7 | −21.3 | Influenza Neuraminidase | 485 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1. A composition-of-matter comprising at least one active moiety surrounded by a scaffold configured for enabling selective influx of an agent capable of interacting with said at least one active moiety.

2. The composition-of-matter of claim 1, wherein said scaffold includes a nucleic acid, a polymer, and/or a silicon structure.

3. The composition-of-matter of claim 2, wherein said nucleic acid structure is a DNA origami structure.

4. The composition-of-matter of claim 1, wherein said agent is a microorganism, a substance or a cell.

5. (canceled)

6. (canceled)

7. The composition-of-matter of claim 1, wherein said scaffold is configured to allow selective influx of an agent having a diameter of 0.01-50 μm.

8. The composition-of-matter of claim 1, wherein said scaffold is configured to allow selective influx of an agent having a diameter of 0.01-0.8 μm.

9. (canceled)

10. The composition-of-matter of claim 1, wherein said active moiety is selected from the group consisting of an antibody, an aptamer, a receptor, a chelator, a ligand, a liposome, nanotube, a dendrimer, a protocell, a cell, a peptide, a protein, an enzyme, a chemical, a detergent, a toxin, a drug and a prodrug.

11. (canceled)

12. (canceled)

13. The composition-of-matter of claim 1, wherein said at least one active moiety is attached to said scaffold.

14. The composition-of-matter of claim 13, wherein said at least one active moiety is attached to said scaffold via a linker.

15. The composition-of-matter of claim 1, wherein said scaffold is substantially non-immunogenic in a vertebrate.

16. The composition-of-matter of claim 1, wherein said scaffold is a non-lipid scaffold.

17. The composition-of-matter of claim 15, wherein said scaffold includes PEG or a PEG derivative, or hyaluronic acid.

18. The composition-of-matter of claim 1, wherein said scaffold forms a particle having an internal lumen and said at least one active moiety is attached to said scaffold in said lumen.

19. (canceled)

20. (canceled)

21. (canceled)

22. (canceled)

23. The composition-of-matter of claim 1, wherein said scaffold further comprises a targeting moiety.

24. The composition-of-matter of claim 23, wherein said targeting moiety is selected from the group consisting of a receptor, an antibody, an aptamer, a tissue-specific moiety, a microorganism-specific moiety, a ligand, and a magnet.

25. (canceled)

26. (canceled)

27. (canceled)

28. The composition-of-matter of claim 4, wherein said active moiety is capable of binding a ligand non-endogenous to said microorganism.

29. The composition-of-matter of claim 1, wherein said scaffold further includes at least one immune-modulating moiety.

30. A pharmaceutical composition comprising the composition-of-matter of claim 1 and pharmaceutically acceptable carrier.

31. The composition-of-matter of claim 1, wherein said active moiety is capable of binding and/or chemically interacting with said agent.

32. A method of isolating an agent from a biological fluid comprising exposing the biological fluid to a composition-of-matter including at least one active moiety surrounded by a scaffold configured for enabling selective influx of the agent capable of interacting with said at least one active moiety, thereby isolating the agent from the biological fluid.

33. (canceled)

34. (canceled)

35. (canceled)

36. (canceled)

37. (canceled)

38. (canceled)

39. (canceled)
